(19) United States
(12) Patent Application Publication
(10) Pub. No.: US 2026/0216511 A1
(43) Pub. Date: Jul. 30, 2026

(54) STIMULATING INFRAHYOID MUSCLE-RELATED TISSUE

(71) Applicant: INSPIRE MEDICAL SYSTEMS, INC., Golden Valley, MN (US)

(72) Inventors: Wondimeneh Tesfayesus, New Brighton, MN (US); Kevin Verzal, Lino Lakes, MN (US)

(73) Assignee: INSPIRE MEDICAL SYSTEMS, INC., Golden Valley, MN (US)

(21) Appl. No.: 19/143,305

(22) PCT Filed: Dec. 22, 2023
(86) PCT No.: PCT/US2023/085822

§ 371 Date: Jun. 25, 2025

Related U.S. Application Data
(60) Provisional application No. 63/477,758, filed on Dec. 29, 2022.

(51) Int. Cl. A61N 1/36; A61N 1/05

(52) U.S. Cl. CPC A61N 1/3606; A61N 1/0553; A61N 1/0556; A61N 1/3601

(57) ABSTRACT

Examples are directed to methods, apparatuses, and/or devices for identifying a target location for stimulating an infrahyoid muscle-related tissue to promote patency of an upper airway of a patient.

### CROSS-REFERENCE TO RELATED APPLICATIONS

This application claims the benefit of the filing date of U.S. Provisional Application Ser. No. 63/477,758, filed Dec. 29, 2022 and entitled “Targeting Stimulation Locations of an Infrahyoid Muscle (IHM)-Innervating Nerve,” the entire teachings of which are incorporated herein by reference.

### BACKGROUND

Sleep disordered breathing, such as obstructive sleep apnea, may cause significant health problems and is common among the adult population. Some forms of treatment of sleep disordered breathing may include electrical stimulation of nerves and/or muscles relating to upper airway patency.

### BRIEF DESCRIPTION OF THE DRAWINGS

FIGS. 1A-1G are diagrams schematically representing an example method and/or device used to implement a method comprising targeting a stimulation location of an infrahyoid muscle-related tissue and an example upper airway of a patient and associated patient anatomy.

FIGS. 2A-2G illustrate an example method for identifying a target location of an infrahyoid muscle-related tissue, such as an infrahyoid muscle-innervating nerve.

FIG. 3 is a flow diagram of another example method for identifying a target location of an infrahyoid muscle-related tissue.

FIG. 4 is a block diagram schematically representing an example implantable medical device.

FIGS. 5A-5E are diagrams schematically representing deployment of example stimulation elements.

FIGS. 6A-6C are diagrams schematically representing patient anatomy and an example device and/or example method for identifying a target location for stimulating an infrahyoid muscle-related tissue.

FIGS. 7A-17EG are diagrams representing example stimulation element or portions thereof.

FIGS. 18A-22B are diagrams schematically representing deployment of example stimulation elements of FIGS. 7A-17GE.

FIG. 23 is a diagram including a front view schematically representing a patient's body, implantable components, and/or external elements of example methods and/or example devices.

FIG. 24 is a schematic diagram of a control portion.

FIG. 25 is a block diagram schematically representing an example sensing portion of an example device and/or used as part of example method.

FIG. 26 is a block diagram schematically representing an example stimulation portion.

FIG. 27A is a block diagram schematically representing an example control portion.

FIG. 27B is a diagram schematically illustrating at least some example arrangements of a control portion.

FIG. 28 is a block diagram schematically representing a user interface.

FIG. 29 is a block diagram which schematically represents some example implementations by which an implantable device may communicate wirelessly with external circuitry outside the patient.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to example apparatuses and/or devices for, and/or example methods of identifying target location(s) for stimulating tissue for therapy and/or other care of medical conditions which may relate to upper airway patency. At least some of the examples comprise use of a medical device in order to increase or maintain upper airway patency. At least some of the examples of the present disclosure may be employed to treat sleep disordered breathing (SDB), which may comprise obstructive sleep apnea (OSA) and/or other types of SDB.

SDB may be treated using a variety of different techniques. In some instances, external breathing therapy devices, such as a continuous positive airway pressure (CPAP) machine or other devices which provide air pressure to the patient during sleep, are used to treat patients. Such external breathing devices may not work for all patients and may be bothersome to the patients, resulting in reduced use. Such patients may sometimes be referred to as being non-compliant or non-adherent because they fail to comply with the prescribed therapy.

For some patients and types of SDB, surgical interventions may be used to improve symptoms, such as uvulopalatopharyngoplasty, lateral pharyngoplasty, lingual tonsillectomy, and tongue reduction surgery, among other procedures. However, such surgical interventions have a mixed record of success for many cases of SDB.

On the other hand, for most patients exhibiting moderate and severe obstructive sleep apnea, great success has been found with the use of some types of implantable neurological devices that provide electrical stimulation to the hypoglossal nerve, which causes the tongue muscle to stiffen and the tongue to protrude, thereby promoting upper airway patency (e.g., dilating the upper airway), sometimes herein referred to as upper airway patency-related tissue, nerves, and/or muscle. Nevertheless, a small percentage of patients may not respond to such treatments and therefore may sometimes be referred to as “non-responders”.

As least some examples of the present disclosure are directed to methods, apparatuses, and/or devices involving identifying and/or accessing a target location for stimulating an infrahyoid muscle (IHM)-related tissue of a patient and which may be used to treat SDB patients which are non-responders to, and/or which are non-compliant with other types of SDB treatments. In some examples, the IHM-related tissue may comprise an IHM-related nerve and/or at least one IHM (e.g., an infrahyoid strap muscle).

In many patients, sleep causes or results in the relaxation of muscles associated with upper airway patency, sometimes herein referred to as “upper airway patency-related muscles.” Sleep also may cause, or result in, other changes that lead to collapse of structures around the upper airway, which may contribute to obstruction of air passage through the upper airway during breathing. Stimulating the IHM-innervating nerve and/or at least one IHM may cause activation of at least one IHM (e.g., infrahyoid strap muscle) and at least cause movement (e.g., pulling) of thyroid cartilage inferiorly in a manner which promotes patency of at least the oropharynx portion of the upper airway. However, accessing a target location of an IHM-innervating nerve and/or an IHM may be difficult due to the physical location of the IHM-innervating nerve, IHM, and/or the particular target location. For example, the IHM-innervating nerve may be located posterior to the omohyoid muscle (OHM) in the neck region, with the OHM overlaying (e.g., superficially or anteriorly to) at least a portion of the sternothyroid muscle (STM) and the sternohyoid muscle (SHM) overlaying (e.g., superficially or anteriorly to) at least a portion of the STM. Additional muscles, such as the sternocleidomastoideoles (SCMMs), may overlie (e.g., superficially or anteriorly) at least portions of the OHM and/or at least portions of other IHMs. In some instances, the target location of the IHM-innervating nerve and/or IHM used to stimulate and promote patency of the upper airway may be located posteriorly (e.g., deep) to the OHM at a location in which the OHM and the IHM-innervating nerve cross or otherwise overlie. In some instances, the target location of the IHM-innervating nerve and/or IHM may be at or near a posterior portion of the SHM and/or STM.

Examples in accordance with the present disclosure are directed to identifying and/or accessing a target location of an IHM-innervating nerve and/or IHM using an access approach that is more efficient and/or superficial as compared to prior approaches.

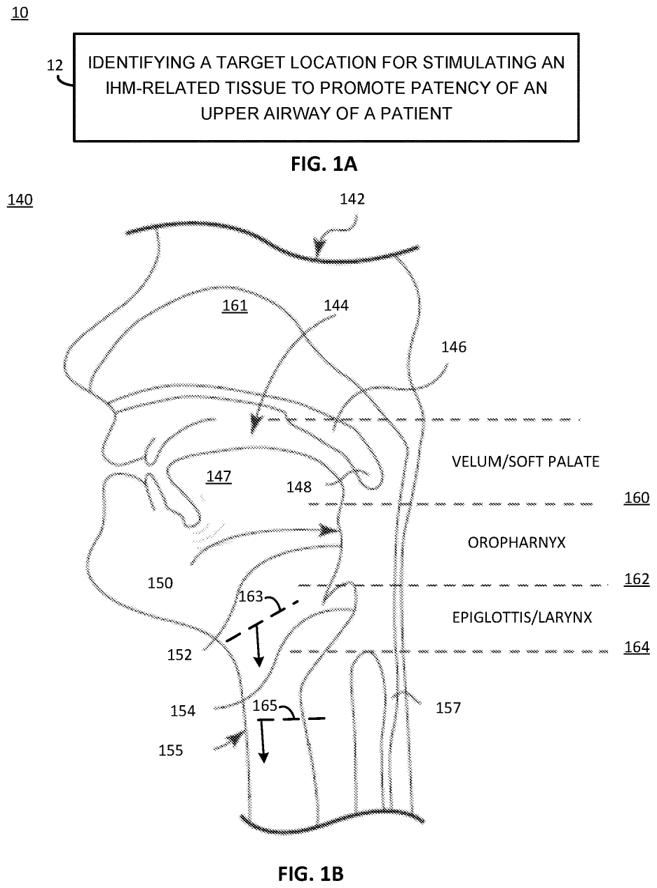

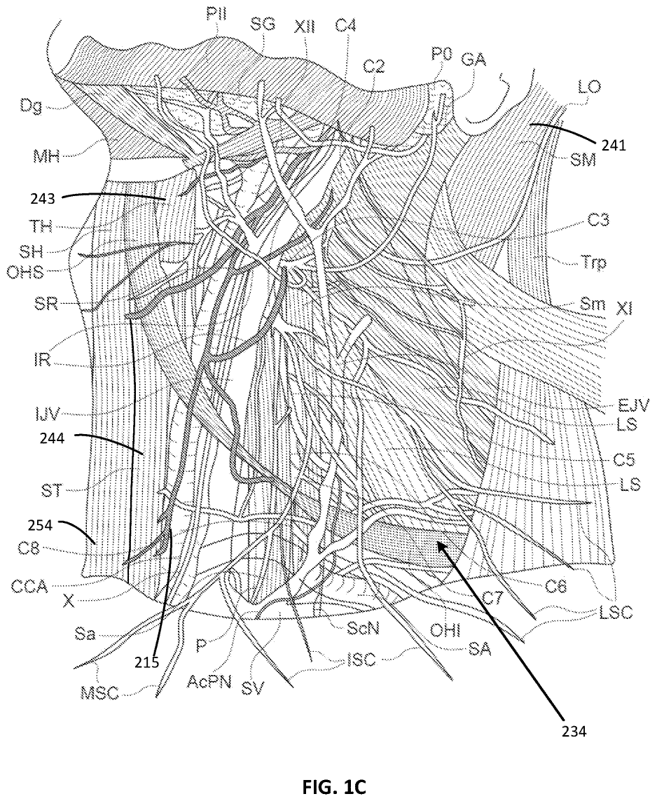

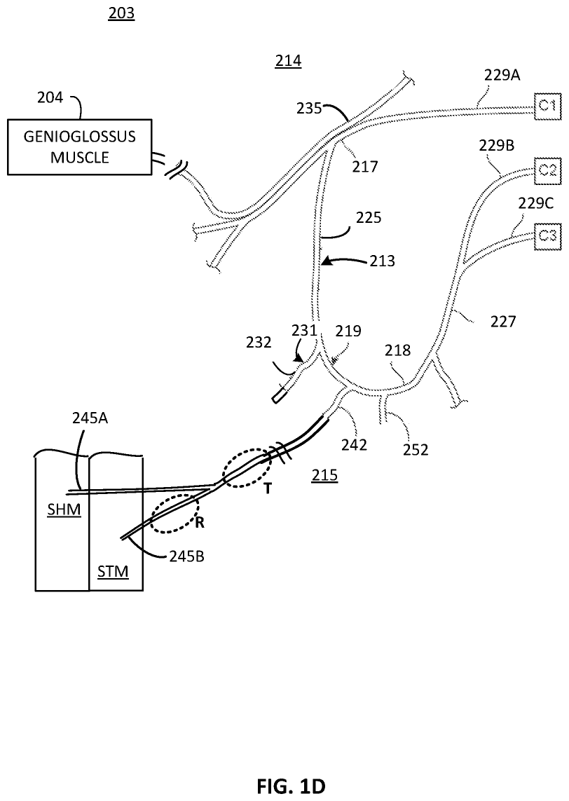

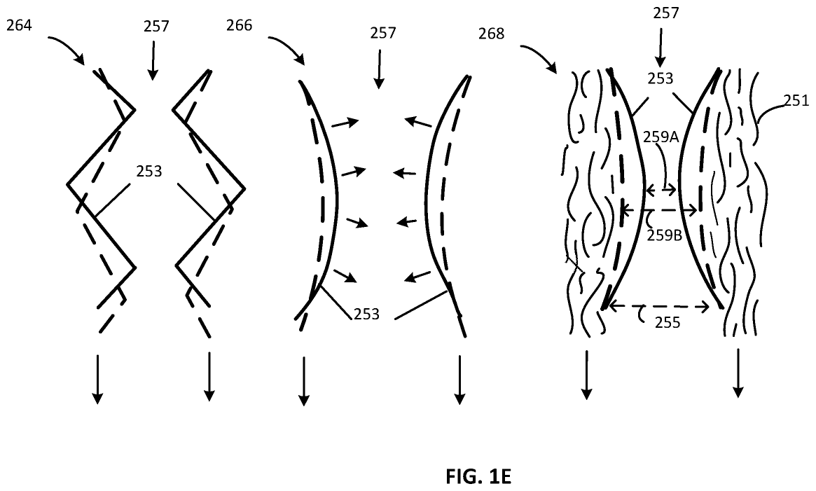

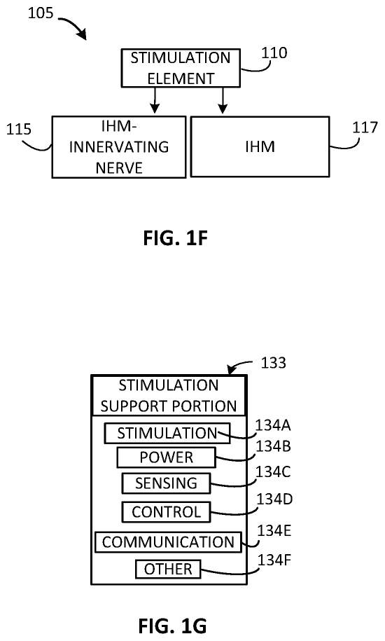

These examples, and additional examples, are described in connection with at least FIGS. 1A-29.

FIGS. 1A-1E are diagrams schematically representing an example method and/or device used to implement a method comprising targeting a stimulation location of an infrahyoid muscle (IHM)-related tissue, such as an IHM-innervating nerve, and an example upper airway of a patient and associated patient anatomy. As shown at 12 in FIG. 1A, the method 10 comprises identifying a target location for stimulating the IHM-related tissue to promote patency of an upper airway of a patient. Among other portions of the upper airway which may be affected, the stimulation at the target location may cause elongation of (e.g., stretching or pulling tension on) at least the oropharynx portion of the upper airway in a manner which causes an increase of, and/or maintains, patency of at least the oropharynx of the upper airway. As further described herein, identifying and/or accessing the target location of the IHM-related tissue may comprise verifying at least one intended (e.g., targeted) physiological response occurs when stimulating at the target location of the IHM-related tissue, such as the IHM-innervating nerve. The physiological response may be associated with promoting upper airway patency. As further described below, the IHM-related tissue may comprise an IHM-innervating nerve, at least one IHM, or both in various examples.

As further described herein, the upper airway includes and/or refers to air-conducting passages of the respiratory system that extend to the larynx from the openings of the nose and from the lips through the mouth. The oropharynx portion of the upper airway may include at least a portion (or all) of the oropharynx that extends approximately from the tip of the soft palate along the base of the tongue until reaching approximately the tip region of the epiglottis.

In some examples, an IHM-innervating nerve may comprise a nerve branch which innervates (directly or indirectly) an infrahyoid muscle (IHM), which may sometimes be referred to as an infrahyoid strap muscle. In general terms, each such IHM-innervating nerve (which innervates at least one IHM) extends from (e.g., originates) from a nerve loop called the ansa cervicalis (AC) or the “AC nerve loop”, which stems from the cervical plexus, e.g., extending from cranial nerves C1-C3. Accordingly, in some examples, at least some of the IHM-innervating nerves may sometimes be referred to as an AC-related nerve in the sense that such nerves/nerve branches (e.g., IHM-innervating nerves) innervating the IHMs do not form the AC nerve loop but extend from the AC nerve loop. Moreover, when stimulation is delivered to at least some locations along the AC nerve loop, at least some IHMs may be activated via the nerve branches which extend from (e.g., off) the AC nerve loop. However, these nerve branches (e.g., extending from the AC nerve loop) may be said to more directly innervate the IHMs. At least because the AC nerve loop is the origin for some nerves which innervate muscles other than the IHMs, some AC-related nerves do not comprise IHM-innervating nerves even though all IHM-innervating nerves may be considered to be an AC-related nerve, in some examples. In some examples, the IHM-innervating nerve may include the neuromuscular junctions of the nerve portions (e.g., fibers or endings) and muscle portions (e.g., IHM).

As further described herein, in some examples, the method 10 may be performed using at least one stimulation element to verify stimulation at the target location of the IHM-related tissue (e.g., IHM-innervating nerve and/or IHM) causes the intended physiological response for promoting upper airway patency. In some examples, the stimulation element may form part of or include an implantable medical device (IMD). In some examples, the stimulation element may comprise at least one stimulation electrode incorporated into a chronically implantable arrangement (e.g., a stimulation electrode arrangement) and/or external removably securable arrangement (e.g., wearable). Such arrangements may be used for treating SDB, such as for sleep apnea. However, in some examples, the stimulation element may comprise a first stimulation element, such as a test tool, used for identifying the target location prior to chronic implantation (or externally removably securing) of a second stimulation element.

Sleep apnea generally refers to the cessation of breathing during sleep. One type of sleep apnea, referred to as OSA, may be characterized by repetitive pauses in breathing during sleep due to the obstruction and/or collapse of the upper airway, and is usually accompanied by a reduction in blood oxygenation saturation. For example, during sleep, upper airway patency-related muscles may not function properly as the muscles become more relaxed, which may cause breathing obstruction as tissue closes in and blocks the upper airway. In some examples, upper airway patency-related muscles include and/or refer to muscles which are involved with promoting (e.g., increasing and/or maintaining) upper airway patency, particularly including patency of at least the oropharynx portion of the upper airway. Some example upper airway patency-related muscles may include IHMs, e.g., sternohyoid (SHM), sternothyroid (STM), thyrohyoid (THM), and/or omohyoid muscle(s) (OHM), at least some of which are innervated by IHM-innervating nerve(s). In some examples, the IHM-innervating nerve innervates the SHM, the STM, and/or the OHM. Some example upper airway patency-related muscles include the genioglossus muscle, which is innervated by the hypoglossal nerve. Examples are not so limited, and in some instances, upper airway patency-related muscles may comprise other muscles, innervated by other nerves.

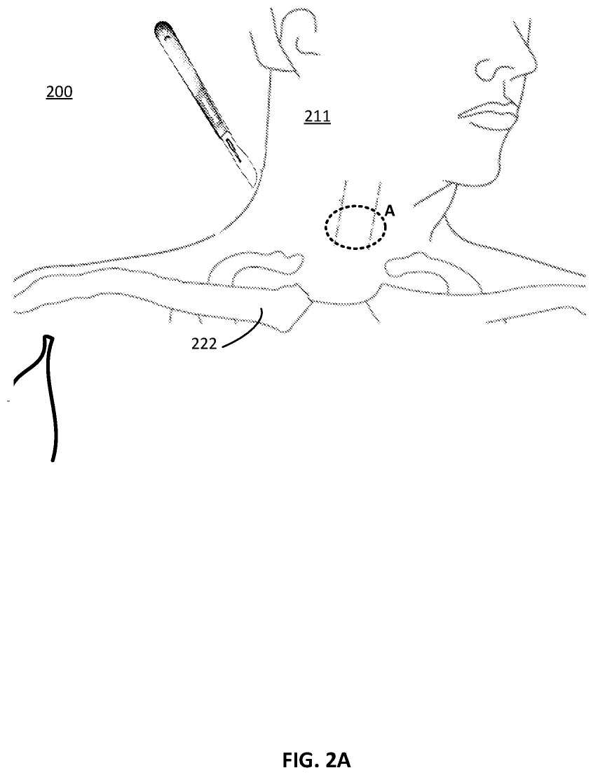

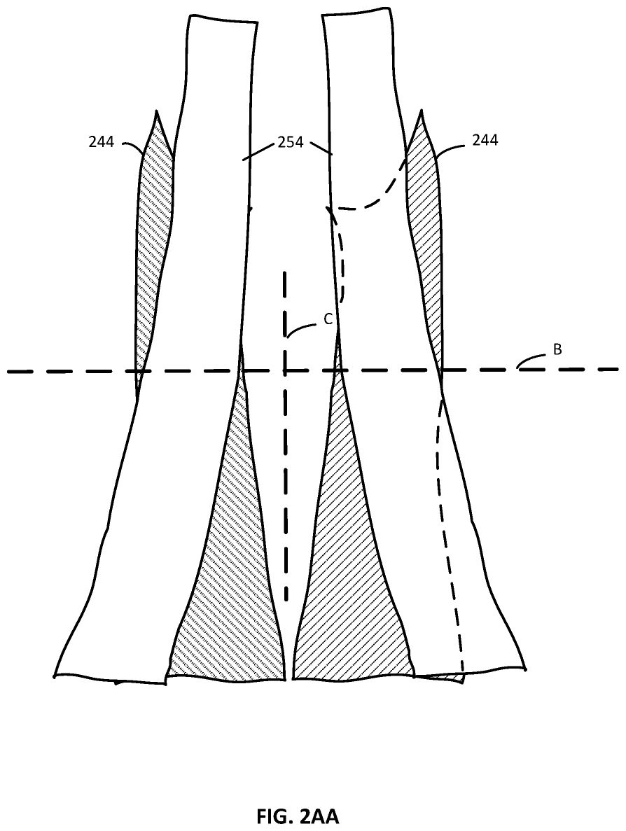

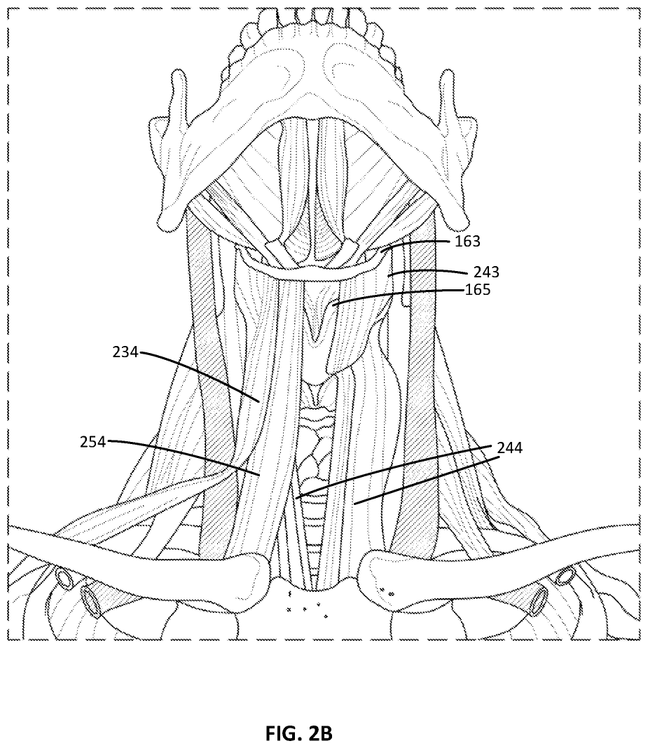

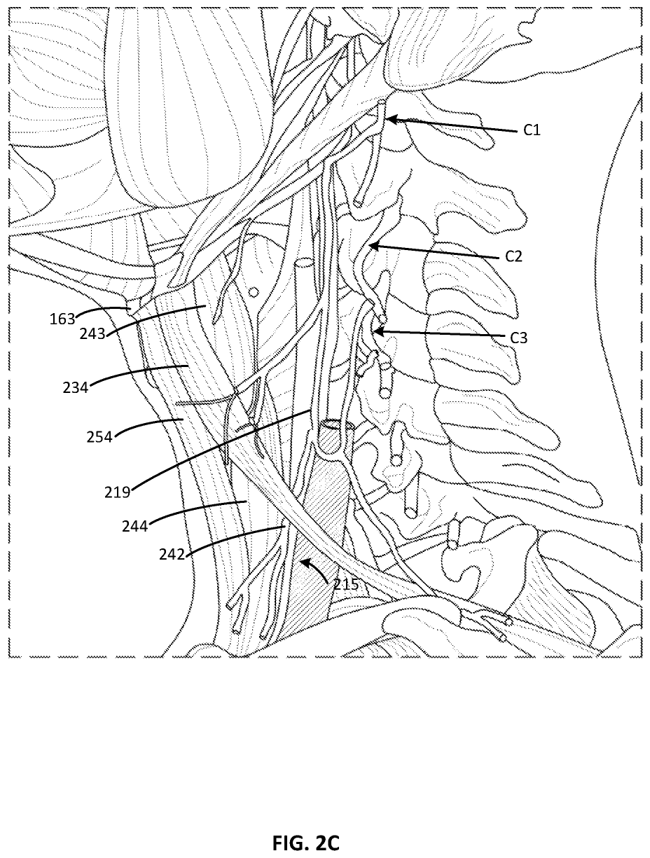

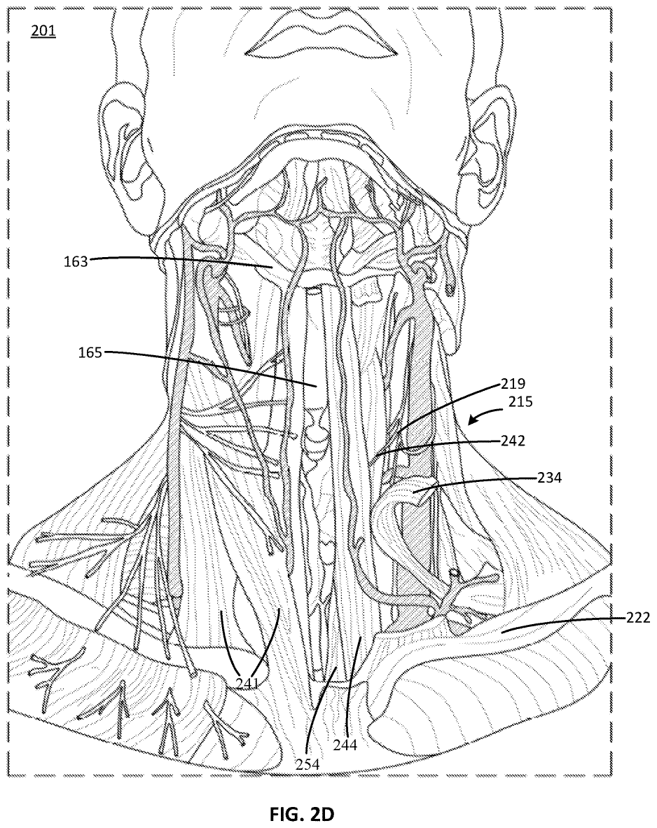

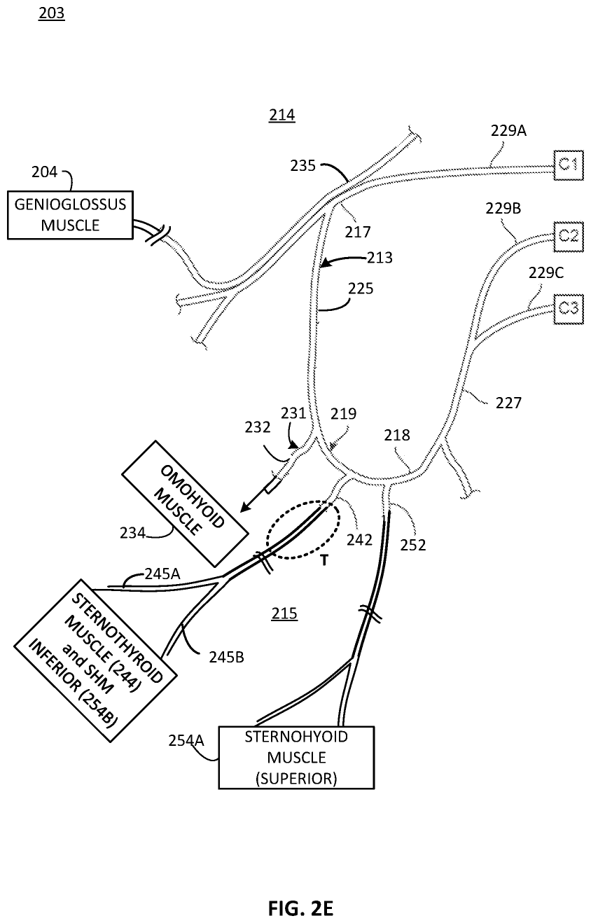

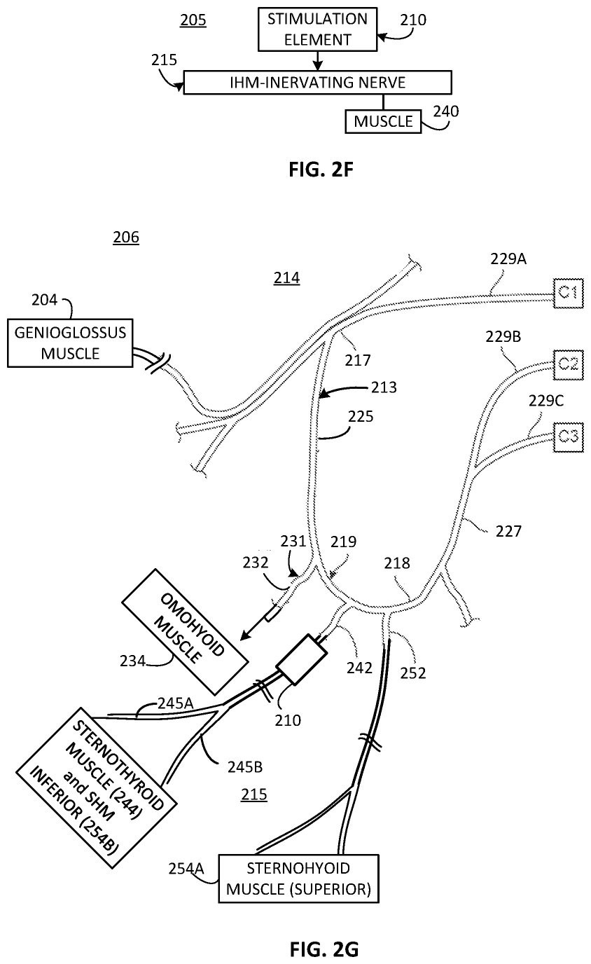

In some examples, a superficial surgical approach may be used to locate the target location of the IHM-related tissue, such as the IHM-innervating nerve, to gain access to the target location, which may be referred to as “an access approach”. As described above, and further illustrated by at least FIGS. 1B-1D, the target location of the IHM-related tissue may be posterior (e.g., deep) to different patient anatomy, including but not limited to the sternocleidomastoid muscle(s) (SCMMs) and/or the OHMs. Further, in some examples, the target location of an example IHM-innervating nerve may be at or near a posterior portion of the SHM and/or STM, such that gaining access to the target location may be difficult. In some examples, the method 10 includes an access approach comprising providing an incision at or near the clavicle at a level associated with the OHM, retracting the OHM superiorly to access the IHM-related tissue, and locating at least a portion of at least one stimulation element at the target location (or a first location) at or near the IHM-related tissue. In some examples, the target location may be medial to the portion(s) of the SCMMs overlaying the OHM(s), as further illustrated and described herein in associated with at least FIGS. 2A-2G.

In some examples, identifying the target location, at 12 in method 10, comprises applying electrical stimulation at the target location and verifying the electrical stimulation applied causes stimulation of at least one IHM (e.g., infrahyoid strap muscle(s)). In some examples, the stimulation to the target location of IHM-related tissue (e.g., an IHM-innervating nerve or the IHM itself) may activate at least one STM. In some examples, the stimulation may activate the STM and the SHM or at least a portion of at least one SHM (e.g., SHM inferior). The verification may include observing activation of the at least one IHM (e.g., infrahyoid strap muscle) responsive to the stimulation applied to the target location of the IHM-related tissue, such as an IHM-innervating nerve. As used herein, stimulating or activating muscle may cause (or include) contraction of the muscle. As further described below, activation of the at least one IHM may cause a physiological response associated with the at least one IHM and, in response to the physiological response, causes a physiological effect associated with promoting upper airway patency. The physiological response may be observed visually and/or otherwise.

In some examples, identifying (and/or accessing) the target location at 12 in method 10 may comprise placing at least a portion of at least one stimulation element at or near a first location of the IHM-innervating nerve and verifying application of stimulation at the first location causes the activation of the at least one IHM. As noted above, the stimulation may activate at least one IHM, such as the STM, and cause a physiological response associated with the IHM and/or other upper airway patency-related tissue. In some examples, the physiological response may include movement of thyroid cartilage inferiorly which may increase or maintain patency of the upper airway, such as at least the oropharynx portion of the upper airway. In some such examples, the increase (or maintenance of) upper airway patency may result from, via the stimulation, at least one physiologic effect such as (but not limited to) displacing tissue (e.g., adipose) within and/or at least partially forming the walls of the oropharynx of the upper airway, sometimes herein referred to as the oropharynx walls or pharyngeal walls (with oropharynx walls being a subset of pharyngeal walls). The displacement of this tissue may reduce extraluminal tissue space in the walls at least partially defining the oropharynx, which reduces extraluminal tissue pressure which would otherwise force the walls of the oropharynx inward to reduce patency. However, by reducing extraluminal tissue pressure, upper airway patency (e.g., oropharyngeal patency) is increased or at least maintained, thereby reducing or preventing SDB. The thyroid cartilage includes and/or refers to tissue in and around at least part of the trachea that contains the larynx, and which is inferior to the hyoid bone.

In some examples, the physiological response may further include movement of the hyoid bone inferiorly, which may impact patency of the upper airway. For example, in addition to activating the STM, in some examples, stimulating at the target location of the IHM-related tissue (e.g., IHM-innervating nerve or multiple IHMs) may further activate at least a portion of at least one SHM. The activation of the SHM may cause the hyoid bone to be pulled inferiorly, which in turn may increase and/or maintain upper airway patency in at least some patients. The hyoid bone is a bone positioned in an anterior midline of the neck between the mandible and thyroid cartilage. Without being bound by theory, it is believed that when the hyoid bone displaces inferiorly, the hyoid bone pulls (e.g., tugs) on the middle pharyngeal constrictor, the stylohyoid muscle, and ligament, which is believed to increase upper airway patency.

The identified (and/or accessible) target location of the IHM-related tissue may be used for treatment of OSA or other types of SDB. The stimulation applied at the target location of the IHM-related tissue may activate the at least one IHM and/or otherwise cause the displacement of tissue at least partially defining walls of the upper airway (e.g., oropharyngeal walls) to maintain patency of at least the oropharynx portion of the upper airway. As described above, some patients may not be compliant with and/or not respond well to various types of treatment for SDB, such as external breathing therapy devices, surgical approaches, and/or delivery of electrical stimulation to the hypoglossal nerve. In some examples, as further described herein, the stimulation applied at or near the target location of the IHM-related tissue may be applied in combination with another treatment, such as the use of external breathing therapy devices and/or electrical stimulation of other upper airway patency-related nerves and/or muscle. In treatment of sleep apnea, increased respiratory effort resulting from the difficulty in breathing through an obstructed airway is avoided in some examples by electrical stimulating at the target location of the IHM-related tissue to move the thyroid cartilage (and/or optionally the hyoid bone) to hold the airway open during at least a portion of the inspiratory phase of breathing. In some examples, the stimulation may be timed relative to (e.g., timed to coincide with at least a portion of) breathing (e.g., an inspiratory phase of each respiratory cycle).

However, examples are not so limited and in some examples, the stimulation at the target location of the IHM-related tissue may be applied independent of sensing respiration and/or obstruction detected.

As further described herein, the method 10 may comprise a number of additional steps and/or variations, such as those illustrated in connection with FIGS. 2A-29.

FIG. 1B is a side view schematically illustrating an example upper airway of a patient. FIG. 1B is a diagram 140 of a side sectional view (cross hatching omitted for illustrative clarity) of a head and neck region 142 of a patient. In particular, an upper airway portion 150 extends from the mouth 144 to a neck portion 155. The upper airway portion 150 includes a velum (soft palate) portion (or region) 160, an oropharynx portion (or region) 162, and an epiglottis-larynx portion (or region) 164. The velum (soft palate) portion 160 includes an area extending below sinus 161, and includes the soft palate 146 approximately to the point at which tip 148 of the soft palate 146 meets a portion of tongue 147 at the back of the mouth 144. The oropharynx portion 162 extends approximately from the tip of the soft palate 146 along the base 152 of the tongue 147 until reaching approximately the tip region of the epiglottis 154. The epiglottis-larynx portion 164 extends approximately from the tip of the epiglottis 154 downwardly to a point above the esophagus 157.

FIG. 1B further illustrates relative location of the hyoid bone 163 and thyroid cartilage 165, as illustrated by dashed lines and with the arrows illustrating the direction of the movement of thyroid cartilage 165, and optionally, the hyoid bone 163, in response to electrical stimulation at the target location of the IHM-related tissue, in accordance with some examples of the present disclosure.

The thyroid cartilage 165 is connected to pharyngeal muscles connected to the pharyngeal walls (such as oropharynx walls) and pulling the thyroid cartilage 165 down effectively causes the pharyngeal walls (e.g., oropharynx walls) to displace and/or redistribute tissue (e.g., at least adipose tissue) in at least the oropharynx portion 162 to reduce extraluminal tissue pressure, which may increase and/or maintain patency of the at least the oropharynx portion of the upper airway 150. For example, the thyroid cartilage 165 may be connected to the inferior pharyngeal constrictor muscle, the stylopharyngeus muscle, and the thyrohyoid muscle.

As shown, the hyoid bone 163 relates to the base 152 of the tongue 147 (e.g., genioglossus muscle). As noted above, and without being bound by theory, it is believed that pulling the hyoid bone 163 inferiorly, as shown by the arrow, may pull on the middle pharyngeal constrictor muscle which effectively increases upper airway patency.

In some examples, moving the hyoid bone 163 inferiorly may elongate (e.g., stretch, tug) at least one at least one pharyngeal constrictor muscle, such as the middle constrictor muscle(s). For example, the middle pharyngeal constrictor muscle may attach to the hyoid bone 163 and depression of the hyoid bone 163 may cause the middle pharyngeal constrictor muscle to elongate (e.g., stretch) and increase airway patency in at least the oropharynx portion 162. In some examples, elongating (e.g., stretching) the at least one pharyngeal constrictor muscle may stiffen the upper airway (e.g., increases pharyngeal muscle tone) and reduce collapsibility of the upper airway. In some examples, the hyoid bone 163 may not move in a purely superior-inferior orientation. As such, as used herein, the hyoid bone 163 being moved inferiorly may include moving generally inferiorly. For example, the patency of upper airway 150 may increase wall stiffness (at least partially defined by pharyngeal muscles) become stiffened/stretched and/or to move in an orientation (e.g., superior-inferior, anterior-posterior, and/or medial-lateral), with such stiffening and/or movement acting to increase patency of the oropharynx portion.

In some examples, and as described above, stimulating the at least one IHM-innervating nerve or at least one IHM at or near a target location may cause a physiological response due to activation (e.g., contraction) of at least one IHM (e.g., infrahyoid strap muscle). The physiological response may include at least one of the thyroid cartilage 165 moving inferiorly and the hyoid bone 163 moving inferiorly, and which causes a physiological effect for treating SDB that occurs remotely from the stimulation and/or remotely from the physiological response, e.g., movement of the thyroid cartilage 165 and/or thyroid cartilage 165 and hyoid bone 163 as described above. In some examples, the physiological effect comprises opening at least the oropharynx portion and/or stiffening of a pharyngeal wall of the patient (which at least partially forms a lumen of the oropharynx portion), which occurs remotely from the physiological response to the stimulation of moving at least the thyroid cartilage inferiorly.

Accordingly, in some examples, the physiological effect occurs a distance away from the stimulation applied at the target location and/or from the physiological response caused by the stimulation. For example, the thyroid cartilage 165 moving inferiorly (and, optionally, the hyoid bone 163 moving inferiorly) in response to stimulation of the IHM-innervating nerve and/or the at least one IHM may occur a distance away from the physiological effect for treating the SDB (which occurs in or near the oropharynx portion 162). The distance way may be a multiple of a diameter of the upper airway 150 of the patient. For example, and as further illustrated by FIG. 1E, the physiological effect may comprise stiffening of a pharyngeal wall (e.g., at least in the oropharynx portion 162) of the patient which occurs remotely from the thyroid cartilage movement action (e.g., near to reference numeral 165).

FIG. 1C shows the IHM-innervating nerve 215 in context with various muscles 234, 241, 243, 244, 254 located in the neck region 105. The muscles 234 may include the IHMs 234, 243, 244, 254 and the SCMMs 241. As shown by FIG. 1C, at least one IHM-innervating nerve(s) 215 extends generally superiorly along the neck region 105, crossing posteriorly (or deeper) to the OHM 234 and innervating (e.g., connecting) at least some of the IHMs 234, 243, 244, 254, e.g., via different branches of the IHM-innervating nerve 215. As further illustrated by at least FIG. 2D, the SCMMs 241 may superficially cross over at least portions of the IHMs 234, 243, 244, 254 and at least a portion of the IHM-innervating nerve 215.

FIG. 1D further shows one example IHM-innervating nerve 215, in context with the IHMs and with the cranial nerves C1, C2, C3. As shown in FIGS. 1D, portion 229A of an AC-main nerve 213 (e.g., a portion or trunk connecting to the AC nerve loop 219) extends anteriorly from a first cranial nerve C1 with a segment 217 running alongside (e.g., coextensive with) the hypoglossal nerve 235 for a length until the a portion of the AC-main nerve 213 diverges from the hypoglossal nerve 235 to form a superior root 225 of the AC-main nerve 213, which forms part of the AC loop nerve 219. As further shown in FIG. 1D, the superior root 225 of the AC nerve loop 219 extends inferiorly (e.g., downward) until reaching near bottom portion 218 of the AC nerve loop 219, from which the AC nerve loop 219 extends superiorly (e.g., upward) to form a lesser root 227 (e.g., inferior root) which joins to the second and third cranial nerves, C2 and C3, respectively and via portions 229B, 229C of the AC-main nerve 213.

As further shown in FIG. 1D, several branches extend off the AC nerve loop 219, including branch 242 which innervates the STM 244 (via branch 245B) and a portion of the SHM 254 (e.g., SHM inferior and via branch 245A). Another branch 252 near bottom portion 218 of the AC nerve loop 219, innervates another portion of the SHM 254 (e.g., SHM superior). In some examples, the collective arrangement of the AC-main nerve 213 (including at least superior root 225 of the AC nerve loop 219) and its related branches (e.g., at least 232, 242, 252) when considered together, or any of those elements individually, may sometimes be referred to as an IHM-innervating nerve 215. It will be further understood that at least one such IHM-innervating nerve 215 is present on both sides (e.g., right and left) of the patient's body.

In some examples, the target location (labeled as “T”) of the AC-related nerve 214 may comprise the branch 242 extending from the AC nerve loop 219 with such branch 242 comprising at least one of the nerve branches innervating the IHMs, such that this nerve branch 242 may be considered one example IHM-innervating nerve 215. For example, the nerve branch (at which target location T is located) extends distally from a superior root portion of the AC nerve loop 219 and innervates at least one of the IHMs 234, 243, 244, 254. In some examples, the at least one IHM comprises the STM 244. In some examples, the at least one IHM comprises the STM 244 and the inferior portion of the SHM 254, sometimes herein referred to as “SHM inferior”. The target location T may be medial to where the SCMMs overlie the OHM 234 (and/or other muscles), such that the SCMMs may not impact accessing the target location T in various examples. Other target locations may be used, such as the target location (labeled as “R”) which includes branch 245B that innervates the STM 244 only and not the SHM 254.

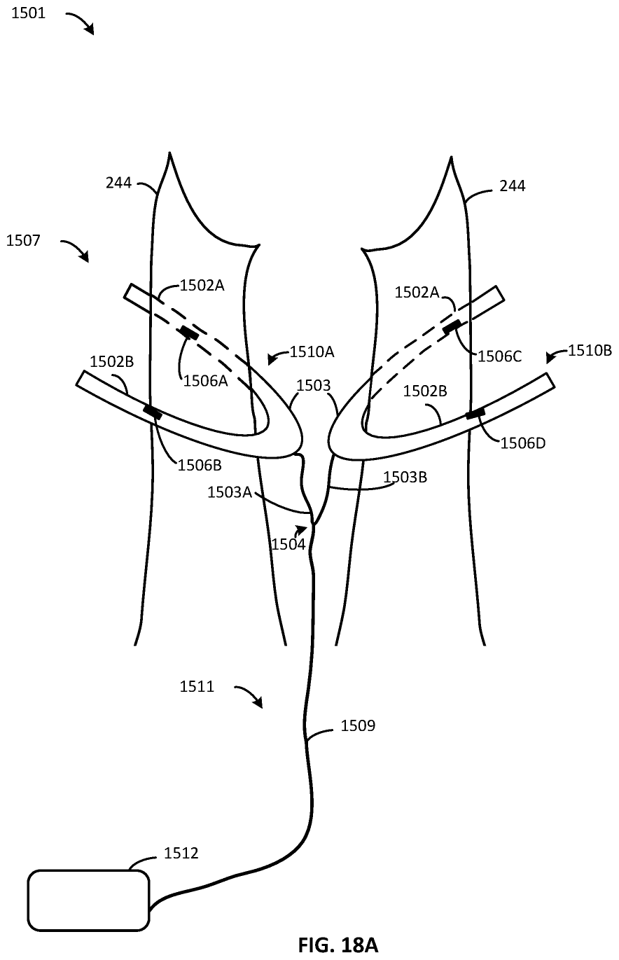

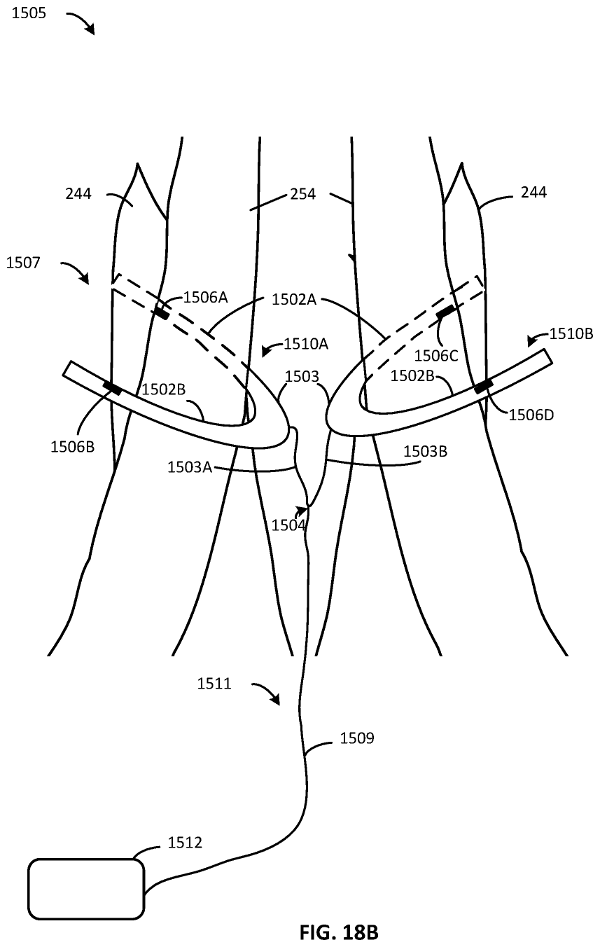

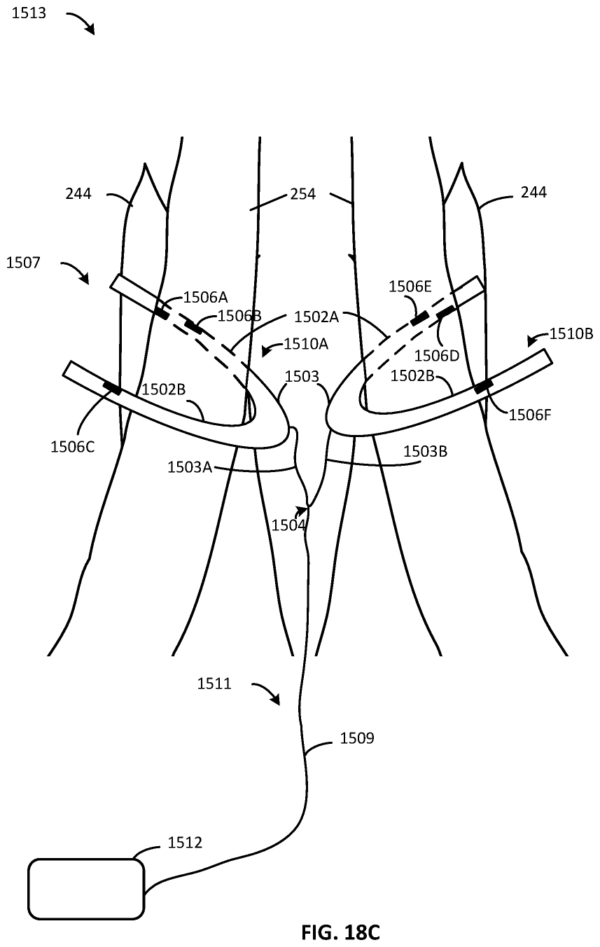

Among other effects, stimulation at the target locations T and/or R of the IHM-innervating nerve 215 acts to bring the larynx inferiorly, which may increase upper airway patency. Other target locations may be used, such as, for example, to stimulate at least one IHM directly and as further illustrated in connection with at least FIGS. 18A-22B.

FIG. 1E are diagrams illustrating different examples of displacing tissue (e.g., adipose, other) within the upper airway responsive to the movement of the at least one of the thyroid cartilage and the hyoid bone, such as in accordance with method 10 of FIG. 1A. The lines in FIG. 1E illustrate walls 253 at least partially defining at least the oropharynx portion of the upper airway, which may be referred to as or include the pharyngeal walls with the base of the tongue defining the anterior portion of the oropharynx portion of the upper airway. In one aspect, the walls 253 are at least partially defined by mucosal lining (skin) over adipose tissue. Accordingly, during the day and/or when the body is functioning properly during sleep, upper airway patency-related muscle (e.g., pharyngeal muscles, other muscles) constrict to displace and/or redistribute the tissue (e.g., at least adipose tissue) in at least the oropharynx portion to reduce extraluminal tissue pressure. This action may increase and/or maintain patency of the upper airway. As may be appreciated, the pharynx, including the oropharynx portion, includes a lumen 257 (e.g., hollow tube) formed by different tissue. FIG. 1E shows a simplified cross-sectional view of a portion of the oropharynx portion with respective tissue forming the lumen 257, as shown (via solid lines) by the walls 253, and other tissue 251 defining or in the intraluminal space, such as connective tissue, adipose tissue, fat and other types of tissue. The tissue forming the walls 253 may include portions of the tongue, tonsils, the soft palate, faucial pillars, the glossotonsillar sulci, uvula, pharyngeal muscles and other muscles, among other types of tissue, which form at least one surface (e.g., walls 253).

As described above, during sleep, at least some of the upper airway patency-related muscles may not function properly as the muscles become more relaxed, which may cause breathing obstruction as some of the tissue (at least partially forming the oropharynx portion) closes in and at least partially blocks the upper airway (in addition to, or instead of at least partial blockage caused by the base of the tongue). The solid lines of FIG. 1E show different examples of obstruction, including pharyngeal unfolding 264, reduced wall conformity 266, and wall/tissue compression 268. In some examples, the physiological response of the at least one of the thyroid cartilage and the hyoid bone due to the contractions of the STM (and optionally the SHM inferior) may cause the physiological effect and as shown by the dashed lines of FIG. 1E, which may be seen as pushing the walls 253 back or stiffening the walls 253 (e.g., pharyngeal walls). As such, the physiological effect caused by the physiological response of the at least one of the thyroid cartilage and the hyoid bone moving may comprise stiffening of at least one pharyngeal wall (e.g., oropharynx walls) of the patient.

As shown by the dashed lines of FIG. 1E, moving the thyroid cartilage inferiorly (and, optionally moving the hyoid bone inferiorly) causes tissue (e.g., at least adipose tissue), which at least partially defines at least the oropharynx portion of the upper airway, to compress (or otherwise be manipulated) to thereby result in a dilation (e.g., an increase in a cross-sectional area 255, 259A, 259B) of at least the oropharynx portion of the upper airway. In the absence of such movement caused by stimulation of an IHM-innervating nerve and/or at least one IHM, at least some of the tissue 251 (e.g., adipose) may cause portions of the wall surface of the oropharynx to protrude into (or otherwise distort, crowd, etc.) the airway passage intended for unobstructed airflow during breathing. For example, the right side of FIG. 1E shows an example of a shortest cross-sectional area of the lumen 257 before stimulating at the target location of the IHM-innervating nerve and/or at least one IHM, at 259A, and after stimulating, at 259B, and also a longer cross-sectional area of the lumen 257, at 255.

Accordingly, in some examples, the cross-sectional area (of at least the oropharynx portion) of the upper airway may increase in response to identifying and stimulating at the target location of the IHM-related tissue (e.g., IHM-16 innervating nerve and/or at least one IHM) in accordance with at least method 10 of FIG. 1A. The cross-sectional area (e.g., 255, 259A, 259B) may include a diameter, a shortest cross-section dimension, and/or a longest cross-sectional dimension. In this way, patency of the upper airway may be increased and/or maintained by the increase in the cross-sectional area.

FIG. 1F is a block diagram schematically representing an example device which may be used to implement the method of FIG. 1A and/or stimulate an IHM-related tissue. Various aspects of stimulation locations, accessing the stimulation locations, control of the stimulation, and IHM-related tissue are further described in associated with at least FIGS. 2A-29. Among these examples, at least stimulation portion 2200 in FIG. 26 provides a general framework for various examples and types of stimulation, as further described later, relative to which the examples of FIG. 1F may be further appreciated.

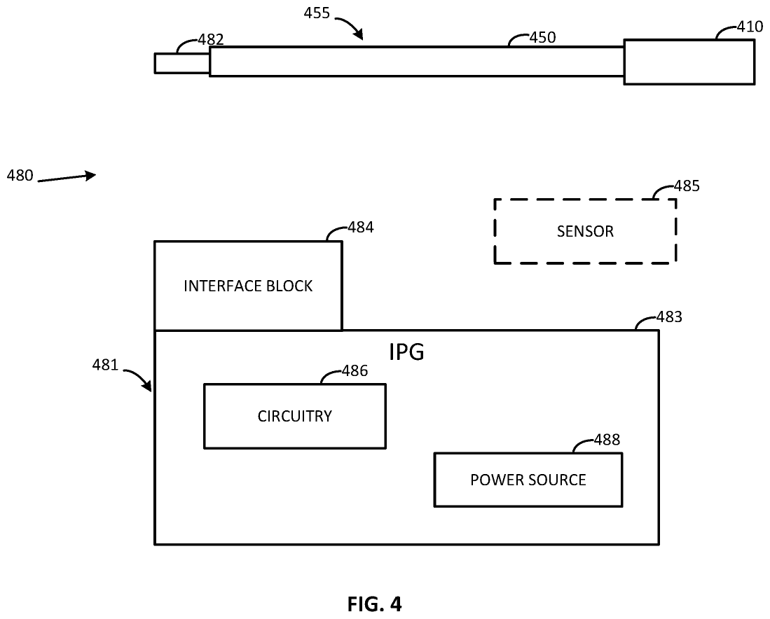

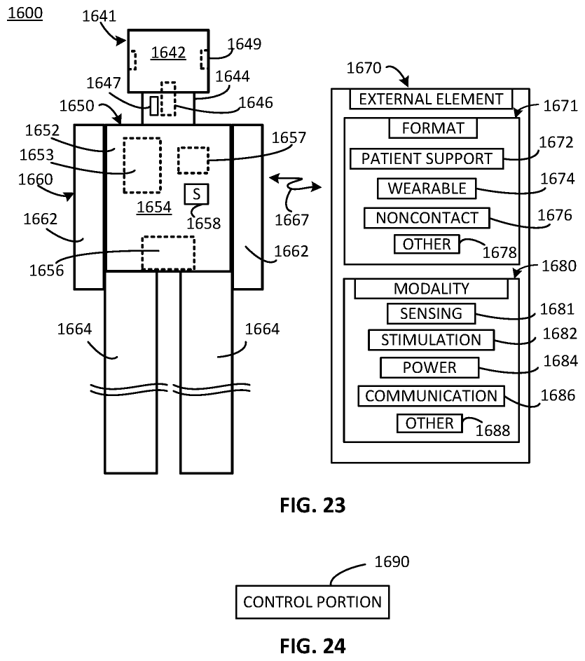

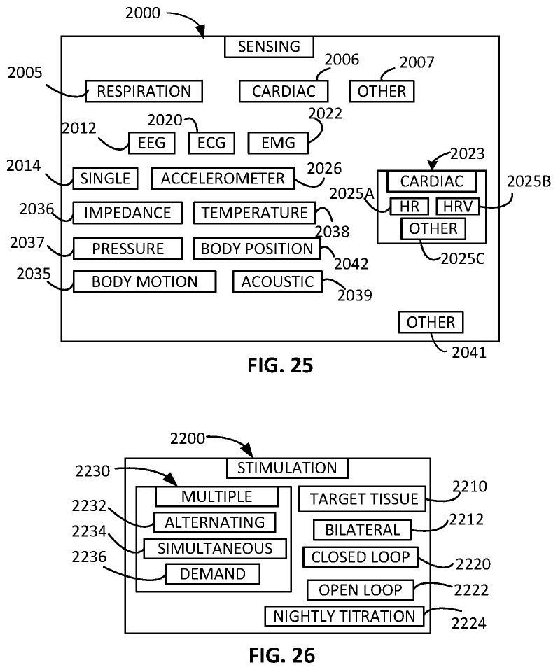

As shown in FIG. 1F, in some examples, a device 105 may comprise a stimulation element 110. In some examples, the stimulation element 110 may include a stimulation electrode arrangement, such as at least one stimulation electrode. In some examples, the stimulation element 110 may further include a lead that supports at least one stimulation electrode (e.g., of a stimulation electrode arrangement) of the stimulation element and include a stimulation support portion (e.g., 133 in FIG. 1G). As described in association with at least FIG. 1G, among other example implementations, one example implementation of a stimulation support portion may comprise stimulation (or control) circuitry, which may be embodied as a pulse generator (e.g., implantable pulse generator (IPG)). Further example implementations of a stimulation support portion may comprise a sensing element to perform sensing and/or to receive sensed data from sensors external to the stimulation element (e.g., including being external to the stimulation support portion), with such sensors being implantable and/or external to the body. In some examples, the sensor(s) may comprise at least some of substantially the same features as described throughout FIGS. 4-22B and/or FIGS. 23-29, with particular reference to sensor portion 2000 of FIG. 25 and/or external element 1670 in FIG. 23.

With further reference to FIG. 1F, in some examples, the stimulation element 110 may form part of a catheter or lead which is placed within the body. Various types of leads may be used, including but not limited to, a spiral-type lead, a basket or lasso type lead, and a lead with tined tips, among others. In general terms, in some examples, the stimulation element 110 (or at least a portion thereof) is located at a position adjacent to upper airway patency-related tissue such as (but not limited to) IHM-innervating nerve 115 and/or an IHM 117, such that stimulation applied via the stimulation element 110 is delivered to the IHM-innervating nerve 115 and/or IHM 117. The tissues may comprise a neuromuscular junction (e.g., motor point) of such nerves and muscles, such as nerve endings or fibers. Via this example arrangement, the stimulation element 110 becomes positioned into stimulating relation to the target upper airway patency-related tissue, e.g., the IHM-innervating nerve 115 and/or IHM 117. In some examples, “stimulating relation” may include and/or refer to a stimulation element 110 (e.g., at least one electrode) being in a position, orientation, and/or distance such that the applied stimulation signal provides at least some capture of a nerve (e.g., at least tone response of muscle, and, in some instances, supra-threshold or full muscle contraction) and/or of a muscle. In some instances, the stimulation may be tonic stimulation, as further described herein.

In some examples, stimulation element 110 may comprise at least one stimulation electrode(s) which may take a wide variety of forms, and may be incorporated within a wide variety of different types of stimulation electrode arrangements, at least some of which are described in association with at least FIGS. 4-22B. In some examples, the stimulation element 110 includes a pair of electrodes or a plurality of pairs of electrodes. In some examples, the stimulation element 110 includes a plurality of ring electrodes. In other examples, the stimulation element 110 includes a planar electrode or a plurality of planar electrodes. In some examples, the stimulation applied may be bipolar or monopolar.

In some examples, the electrode(s) of the stimulation element 110 used for applying stimulation also may be used for sensing, but not necessarily for simultaneous stimulation and sensing. However, in some examples, the electrode(s) of the stimulation element 110 are used solely for applying stimulation while some electrode(s) may be used solely for sensing.

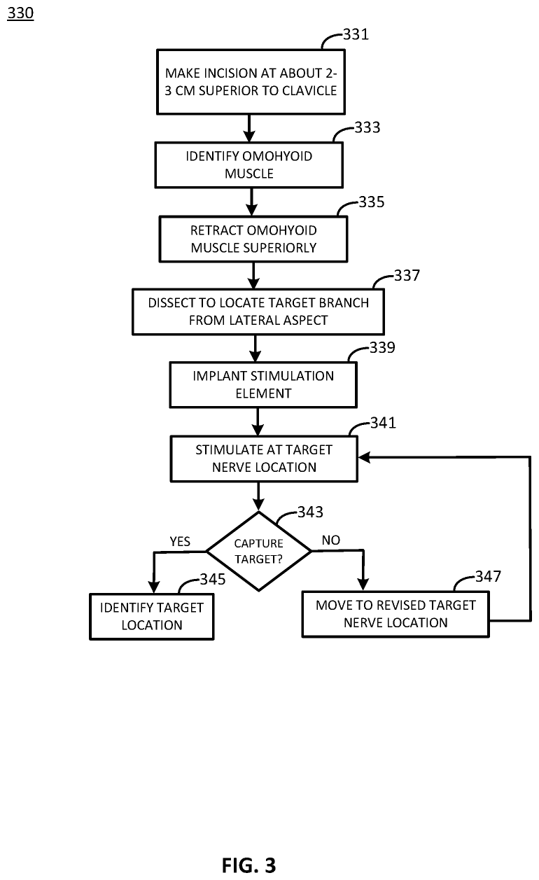

In some examples, the device 105 may be implanted within the patient's body. For example, the stimulation element 110, or at least a portion thereof, may be inserted within the patient's body and maneuvered to the target location for applying stimulation to the IHM-innervating nerve 115 and/or IHM 117, as further described in connection with at least FIGS. 2A-3. In some examples and as noted above, the stimulation element 110 of the device 105 may further include a lead that supports the at least one stimulation electrode.

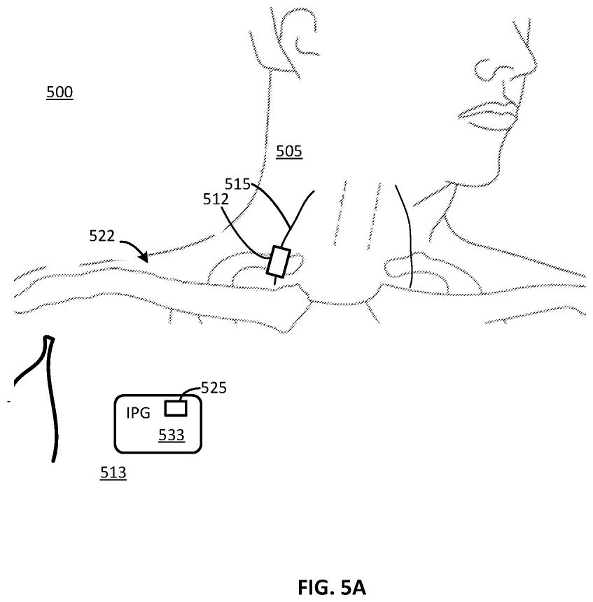

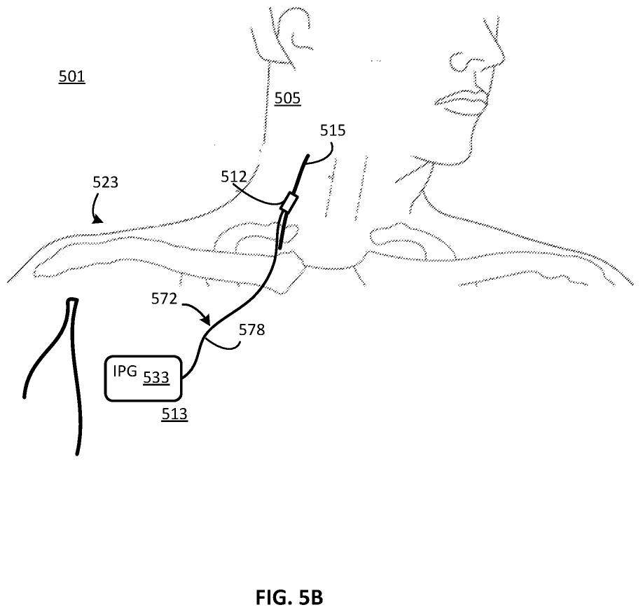

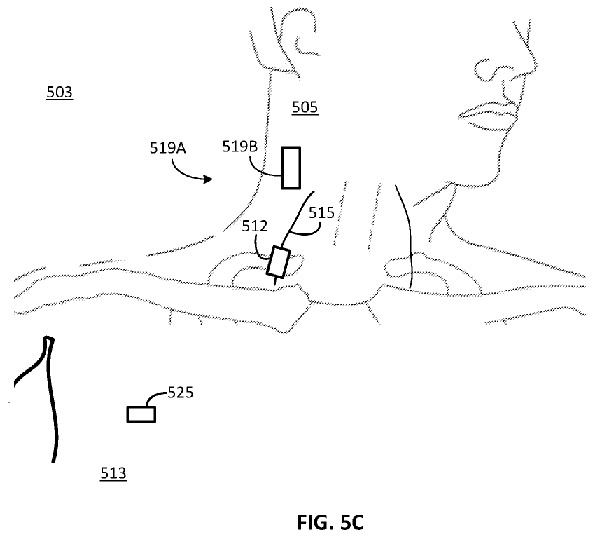

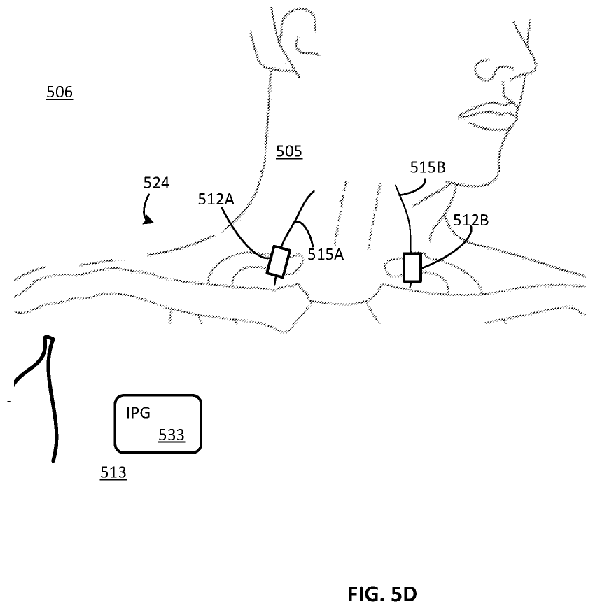

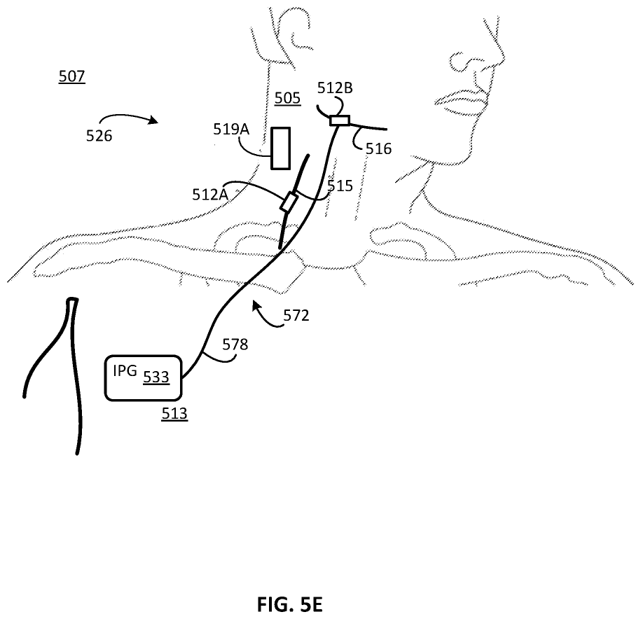

In some examples, as noted above, the stimulation element 110 may further include a stimulation support portion (e.g., at least 133 in FIG. 1G) which may be embodied as a pulse generator (PG), such as illustrated in connection with at least FIGS. 4A-5E. In some such examples, the entire PG (and/or other power, control, and/or communication elements) may be implantable while in some examples, some portions of the PG (and/or other power, control, and/or communication elements) may be external to the patient as further described in association with at least FIG. 23. In some examples, the IPG or a non-implanted PG may be separate from the stimulation electrode arrangement. In some examples, the pulse generator may be located within the head-and-neck region or the pectoral region of the patient. In some examples, the IPG may be chronically implanted in at least one of the torso region, the neck region, or the cranial region. The torso region may include the sternum, pectoral region, or other areas. The neck region may include the neck and other areas, such as a transitional area of the neck (e.g., between the neck and torso, and/or between the neck and cranial region) including the clavicle, manubrium (e.g., at top of sternum), and mandible. In some examples, the cranial region may include the skull, such as behind the ear of the patient, among other locations. In some examples, components may be implanted in the cranial region or in the head region, which may be referred to as a “head-and-neck region” for ease of reference.

In some examples, the stimulation element 110 may include a stimulation support portion, such as further described herein in connection with at least FIG. 1G. As further described herein, in some examples the stimulation support portion may be implemented as a PG, such as an IPG.

As shown in FIG. 1G, in some examples, the stimulation support portion 133 may comprise stimulation function circuitry 134A, a power element 134B, a sensing element 134C, a control element 134D, a communication element 134E (e.g., at least a receiver), and/or other element 134F.

In some examples, the stimulation function circuitry 134A may comprise passive stimulation circuitry, e.g., circuitry which does not generate a stimulation signal but which may receive a stimulation signal generated elsewhere (e.g., external of the patient or from an implanted device) and which is then communicated (e.g., via lead) to the electrodes of the stimulation electrode arrangement for stimulating the IHM-innervating nerve 115, IHM 117, and/or other upper airway patency-related tissue.

With further reference to the particular example illustrated in FIG. 1G, in some examples, the stimulation function circuitry 134A comprises active stimulation circuitry, e.g., components sufficient to generate a stimulation signal within the stimulation support portion 133 for transmission (e.g., via a lead or other means) to the electrodes of the stimulation electrode arrangement of the stimulation element (e.g., 110 of FIG. 1F). In some such examples, the stimulation support portion 133 may sometimes comprise and/or be referred to as a PG. Moreover, in some such examples, given the stimulation support portion 133 being sized and shaped for implantation in the head-and-neck region, the stimulation support portion 133 may sometimes be referred to as a microstimulator.

Whether referred to as a microstimulator or not, in these examples the housing 135 of the stimulation support portion 133 may sealingly contain (e.g., encapsulate) the stimulation function circuitry 134A, along with other elements such a power element 134B, communication element 134E, and/or control element 134D, among other potential components (e.g., sensing 134C, etc.).

In some examples, the stimulation support portion 133 of the stimulation element 110 may comprise a power element 134B. The power element 134B may be non-rechargeable, in some examples. However, the power element 134B may be re-chargeable in some examples such that the power element 134B receives power from a power source external of the stimulation support portion 133, with the power source being implantable in some examples or being external of the patient in some examples. For instance, the power element 134B may receive power via a wired connection (e.g., in some examples in which the power source is implantable) or via wireless communication, in which the power source may be implantable or external to the patient. In some examples in which the power source may be external to the patient, the power source may comprise at least some of substantially the same features and attributes as external power portion 1684 in FIG. 23, as further described below.

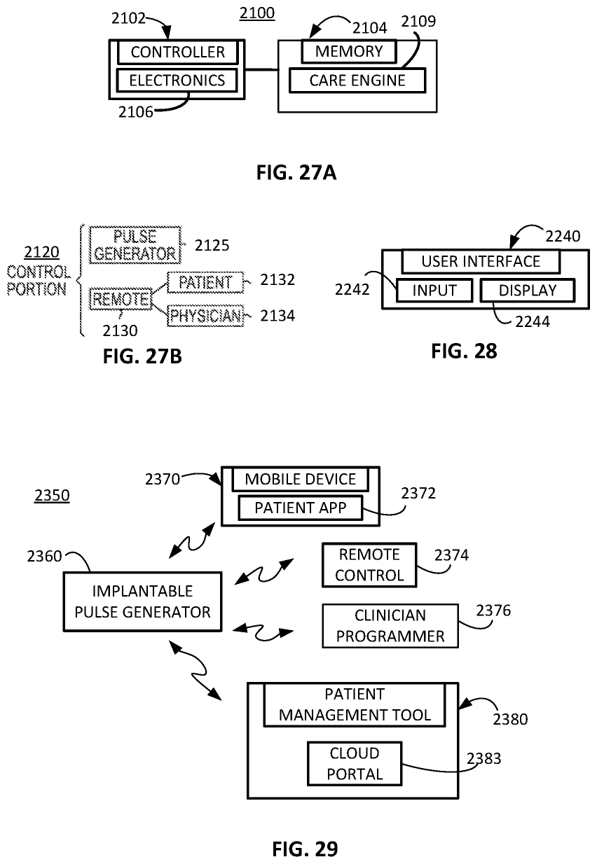

In some examples, the stimulation support portion 133 comprises a control element 134D which provides on-board control of at least some of the functions of the stimulation element 110 (including stimulation electrode arrangement, stimulation support portion 133, and/or other components of the stimulation element 110). In some examples, the control element 134D may comprise the entire control portion for the stimulation element 110. In some examples, the control element 134D may form part of a larger control portion in which the control element 134D may receive at least some control signals from components of the control portion external to the stimulation support portion 133. In some such examples, these components of the control portion which are external to the stimulation support portion 133 also may be external to the patient. For example, the control element 134D of stimulation support portion 133 may comprise at least a partial implementation of, and/or communicate with, a control portion 1690 of FIG. 24 and/or control portion 2100 of FIG. 27A. As such, consistent with the later described control portion 2100 of FIG. 27A, the control element 134D in FIG. 1G also may comprise a memory to store stimulation therapy information (e.g., therapy settings, usage, outcomes, etc.), control information, sensed information (per sensing element 1034C), etc.

In some examples, the sensing element 134C of stimulation support portion 133 may store data sensed by an on-board sensor of the stimulation element 110 and/or sensed via sensor external to the stimulation element 110 (e.g., external to stimulation support portion 133, stimulation electrode arrangement) with such sensor (external to the stimulation element 110) being implantable or external to patient. In some examples, an on-board sensor may comprise an accelerometer (e.g., tri-axis), gyroscope, etc. In some examples, such on-board sensor may comprise an electrode exposed on surface of housing, which in combination with other electrodes may be used to sense impedance and/or other biosignals. With these brief examples in mind, it will be understood that in some examples the sensing element 134C may comprise, and/or receive sensed information from, at least some of substantially the same sensing elements, functions, etc. as later described in association with at least FIG. 23 (e.g., external element 1670), FIG. 25 (e.g., sensing portion 2000), FIG. 26 (e.g., stimulation portion), and/or FIG. 27A (e.g., control portion 2100, care engine 2109).

In some examples, the stimulation support portion 133 of the stimulation element 110 may comprise a communication element (e.g., coil, antenna and any related circuitry) to transmit and/or receive the control information, therapy data, sensed data, and the like. In addition to, or instead of these examples, the communication element may be configured to facilitate receive power from a power source(s) external to the stimulation support portion 133, whether via wired connection or wirelessly. In some examples, the communication element 134E may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), ultrasound, and/or other short range or long range wireless communication protocols suitable for use in communicating between implanted components within the body and/or communicating between implanted components and external components in a medical device environment.

It will be understood that in some examples of the present disclosure, a lead may be omitted and at least some of the operative components of the stimulation support portion 133 may be incorporated into and/or with the stimulation electrode arrangement, such as illustrated by (but not limited to) the example stimulation element 512 of FIG. 5C. In some such examples, the stimulation electrode arrangement may sometimes comprise, or be referred to as, a leadless stimulation electrode arrangement or a leadless stimulation element 110. In some of these examples, the functions and/or components of the stimulation support portion 133 which are incorporated into the stimulation electrode arrangement may comprise passive stimulation circuitry (which may be embodied as a part of the communication element 134E) to receive a stimulation signal generated elsewhere and conduct this stimulation signal to the electrodes of the stimulation electrode arrangement.

Referring back to FIG. 1F, in some examples, portions of the stimulation element 110 may comprise a fixation arrangement which acts to maintain at least the stimulation electrode arrangement in a selected location (within the patient's body) to maintain at least some electrodes of the stimulation element 110 in stimulating relation to the targeted portion of the IHM-innervating nerve 115 and/or IHM 117. Moreover, in some such examples, the housing of stimulation support portion (133 of FIG. 1G) may comprise the fixation arrangement, which may comprise multiple fixation elements (e.g., tines, barbs, and/or other tissue-engaging structures to fixate (e.g., hinder or prevent movement of) the housing relative to target tissue in/at which the stimulation support portion is present. In some examples, with or without such a fixation arrangement (e.g., tines, barbs, etc.), a shape of the housing of the stimulation support portion may act to help fixate the housing relative to surrounding tissues.

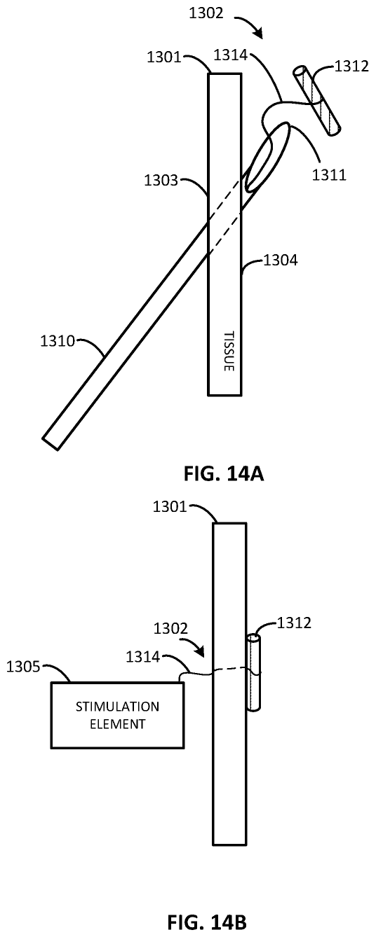

At least some fixation arrangements may be implemented according to any one of the examples described in association with at least FIGS. 14A-17EG.

FIGS. 2A-2G illustrate an example method for identifying a target location of an IHM-related tissue, such as an IHM-innervating nerve. The method illustrated by FIGS. 2A-2G may include an example implementation of the method 10 of FIG. 1A, the target location and stimulation as illustrated and described in associated with FIGS. 1B-1E, and/or may be implemented using the device 100 of FIGS. 1F-1G. More particularly, the method of FIGS. 2A-2G illustrates an example access approach used to identify and access a target location of an IHM-related tissue and verify stimulation at the target location promotes upper airway patency.

As shown at 200 in FIG. 2A, the method comprises providing an incision at or near a clavicle 222 of a patient at a level associated with the OHM. The incision may be provided in a neck region 211 of the patient, as illustrated by the target incision location A. In some examples, the target incision location A may be about two to about three centimeters superior (rostral) to the clavicle 222 and at approximately the level of the OHM.

FIG. 2AA illustrates further example target incision locations B, C. As shown, target incision location B is transverse to the anterior midline (e.g., sagittal) and is superior to the clavicle and at approximately a level where the SHM 254 and STM 244 cross one another. Target incision location C is along (e.g., parallel) to the anterior midline of the patient, which may be between the SHM 254 and STM 244 and may coincide or extend along a portion where the SHM 254 and STM 244 cross one another.

FIGS. 2B-2C illustrate the IHM-innervating nerve 215 in context with various muscles 234, 243, 244, 254 located in the neck region. More particularly, FIG. 2B illustrates a front view of the neck region of the patient and the IHMs 234, 243, 244, 254 located in the neck region, including the OHM 234 which overlies at least a portion of the SHM 254 and the STM 244, as previously described in connection with at least FIG. 1C. The SCMMs 241 may further overlie superficially to various portions of the IHM 234, 243, 244, 254 (e.g., infrahyoid strap muscles). Further illustrated by FIG. 2B is the thyroid cartilage 165 and the hyoid bone 163.

FIG. 2C illustrates a side view of the neck region of the patient and shows the location of the IHM-innervating nerve 215 with respect to the IHMs 234, 243, 244, 254 located in the neck region. As shown by FIG. 2C, the OHM 234 overlies at least one portion of the IHM-innervating nerve branch 242, which extends from the AC nerve loop 219. As previously stated, the AC nerve loop 219 less directly innervates the STM 244 and the IHM-innervating nerve branch 242 more directly innervates the STM 244. In some examples, the IHM-innervating nerve branch 242 is an example AC-related nerve.

As shown at 201 in FIG. 2D, the method may further comprise retracting the OHM 234 superiorly. For example, the OHM 234 may be identified and retracted superiorly to provide access to the portion of the IHM-innervating nerve 215, e.g., a portion of the IHM-innervating nerve branch 242, which the OHM 234 overlies.

FIG. 2D includes a more-detailed illustration of the patient anatomy from FIG. 1D, and may include at some of substantially the same features and attributes as previously described in connection with FIG. 1D, as illustrated by the common numbering. The already described common features and attributes are not repeated for ease of reference.

The method may further comprise dissecting deep to the OHM 234 to locate the branch 242 of the IHM-innervating nerve 215 which innervates at least the STM 244 from the lateral aspect, herein sometimes referred to as the “IHM innervating nerve branch 242” and such as shown by the side views of at least FIGS. 1C-1D. For example, as shown at 203 of FIG. 2E, the method may comprise dissecting deep to identify the target location T along IHM-innervating nerve branch 242 of the IHM-innervating nerve 215. Examples include other target locations, such as target location R illustrated by FIG. 1D.

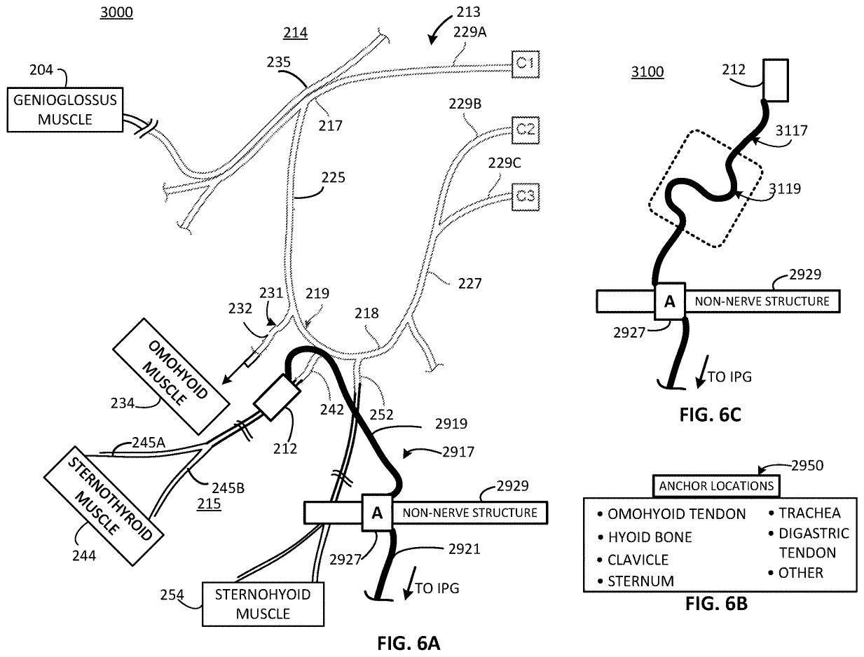

FIG. 2E (as well as FIG. 2G and FIG. 6A as further illustrated and described herein) includes a more simplified illustration of the patient anatomy from FIGS. 1D and 2D, and may include at some of substantially the same features and attributes as previously described in connection with FIGS. 1D and 2D, as illustrated by the common numbering. The already described common features and attributes are not repeated for ease of reference.

As previously described, portion 229A comprises an AC-main nerve 213 extending anteriorly from a first cranial nerve C1 with a segment 217 running alongside the hypoglossal nerve 235 until the AC-main nerve 213 diverges from the hypoglossal nerve 235 to form a superior root 225, which forms part of an AC nerve loop 219. A portion of the hypoglossal nerve 235 extends distally to innervate the genioglossus muscle 204. The superior root 225 extends inferiorly until reaching near bottom portion 218 of the AC nerve loop 219, from which the nerve loop 219 further extends superior to form a lesser root 227 to complete the AC nerve loop 219, which joins to the second and third cranial nerves, C2 and C3, respectively.

As previously described, several branches 231 extend off the AC nerve loop 219, including branch 242 which innervates the STM 244 and a portion of the SHM, e.g., SHM inferior 254B. Another branch 252, near bottom portion 218 of the AC nerve loop 219, innervates another portion of the SHM, e.g., SHM superior 254A. The branches 231 further include branch 232 which innervates the OHM 234. The AC-related nerve 214 may include additional branches (beyond those illustrated and described) extend from the AC nerve loop 219.

In some examples, the target location T is located along branch 242, which extends distally from a superior root 225 of the AC nerve loop 219 and innervates at least the STM 244. Accordingly, at least branch 242 may sometimes be referred to as an IHM-innervating nerve, as previously noted. Stimulating at the target location T may be used to completely capture and/or fully activate the STM 244 and/or to promote upper airway patency. In some examples, target location T of the IHM-innervating nerve 215 may innervate the STM 244 and the SHM inferior 254B. Stimulated at target location T may thereby fully activate the STM 244 to pull the thyroid cartilage inferiorly, via branch 245A, and, optionally activate the SHM inferior 254B to pull the hyoid bone inferiorly, via branch 245B. Activating the SHM superior 254A alone or activating a combination of the SHM superior 254A and SHM inferior 254B may have greater impact on hyoid bone movement (inferiorly) than activation of the SHM inferior 254B without activating the SHM superior 254A. As such, in some examples, activating the SHM inferior 254B may have minimal (or below a threshold) impact on the movement of the hyoid bone.

While stimulation of just the hypoglossal nerve 235 (or some branches thereof) may be effective in increasing upper airway patency to a sufficient degree to ameliorate OSA in high percentage of qualified patients (e.g., least about 70 to 80% in some examples) when using certain types of implantable neurostimulation devices, some patients may benefit from stimulation of the an IHM-innervating nerve (e.g., 242, 219) in addition to, or instead of, stimulation of the hypoglossal nerve 235. Moreover, for a particular patient, certain positions of the head-and-neck and/or of their body (e.g., supine, lateral decubitis, etc.) may be treated more effectively by stimulating an IHM-innervating nerve (e.g., more direct via branch 242 or less directly via AC nerve loop 219), with or without stimulation of the hypoglossal nerve 235.

In some examples, the method comprises use of at least one stimulation element 210 as shown at 205 of FIG. 2F. More particularly, the method may comprise stimulating the IHM-innervating nerve 215 at or near the target location T of the IHM-innervating nerve branch 242 via the at least one stimulation element 210, and at least one IHM 240 innervated by the IHM-innervating nerve 215 may be activated in response to the stimulation. In some examples, the method may comprise stimulating other tissue, such as at least one IHM, as previously described, and which may be stimulated using target locations such as those further illustrated in connection with FIGS. 18A-22B.

For example, as shown at 206 in FIG. 2G, the method may comprise locating at least a portion of the at least one stimulation element 210 at a first location at or near at least one IHM-innervating nerve 215, e.g., branch 242. In some examples, the target location (e.g., T) may be along the IHM-innervating nerve branch 242 (among other possible locations distally, proximally along that IHM-innervating nerve branch 242 and/or AC nerve loop 219) and a verifying application of stimulation at the first location causes activation of at least one IHM, such as by applying the electrical stimulation at the first location. In response to applying the stimulation, the method may comprise verifying the electrical stimulation applied causes activation of the at least one IHM, such as the STM 244 (and optionally, the SHM inferior 254B), by observing a physiological response associated with the IHM and/or of at least one upper airway patency-related tissue (e.g., thyroid cartilage). As previously described, the physiological response may comprise at least movement of thyroid cartilage inferiorly, which may promote upper airway patency. In some examples, the physiological response comprises movement of thyroid cartilage inferiorly and optionally movement of the hyoid bone inferiorly. In other examples, the method may comprise locating at least a portion of the at least one stimulation element 210 at a first location at or near the STM 244 and/or SHM, applying stimulation, and verify the intended activation and/or physiological response occurs.

In some examples, upper airway patency-related tissue includes and/or refers to tissue, and which may be involved with upper airway patency. Example upper airway patency-related tissue includes muscle, nerves, tendons, ligaments, bone, cartilage, among other tissue, such as tissue forming pharyngeal walls. Example upper airway patency-related muscles include the IHMs (e.g., infrahyoid strap muscles), stylopharyngeus muscle, pharyngeal constrictor muscles, and the genioglossus muscle, as previously described. Example upper airway patency-related nerves include the IHM-innervating nerves, and the hypoglossal nerve. Examples are not so limited, and in some instances, upper airway patency-related tissue may comprise other muscles, other nerves, and/or other types of tissue, such as the thyroid cartilage and hyoid bone. In some examples, stimulation of upper airway patency-related tissue may comprise stimulation of upper airway patency-related muscle(s), upper airway patency-related nerve(s), or a combination of some muscle(s) and nerve(s).

In some examples, the first location is identified as the target location in response to verification of the stimulation at the first location causing activation of the at least one IHM. For example, the verification may comprise applying the stimulation to the target location of an IHM-innervating nerve branch (e.g., 242) (and/or more distal or more proximal location considered an IHM-innervating nerve) and identifying at least displacement of the thyroid cartilage inferiorly. As previously described, stimulating the IHM-innervating nerve (e.g., branch 242) at the target location may cause the thyroid cartilage to move inferiorly via activation of at least one IHM, e.g., the STM 244. In some examples, in addition to the thyroid cartilage displacing inferiorly, the hyoid bone may displace inferiorly via the activation of the at least one IHM, e.g., the SHM inferior. The thyroid cartilage (and optionally, the hyoid bone) moving (e.g., displacing) inferiorly may cause an increase of or maintaining of patency of at least the oropharynx portion of the upper airway, such as by longitudinally elongating (e.g., stretching) the upper airway via the movement of the thyroid cartilage inferiorly.

The first location may be identified as the target location in response to the verification of the stimulation at the first location causing the activation of the at least one IHM. In some examples, the stimulation may not cause activation of the at least one IHM or may otherwise not cause the intended physiological response. In response to not causing the activation of the at least one IHM and/or the intended physiological response, the at least portion of the at least one stimulation element 210 may be moved to a second location.

In some examples, the at least portion of the stimulation element 210 may be moved to the second location in response to at least one of: (i) the stimulation not causing activation of the at least one at least one IHM, (ii) the stimulation causing activation of the at least one at least one IHM without causing a physiological response of at least one upper airway patency-related tissue (e.g., thyroid cartilage moving inferiorly), and (iii) the stimulation causing activation of the at least one at least one IHM that causes the physiological response of at least one upper airway patency-related tissue of a patient below a threshold (e.g., the thyroid cartilage moves less than the threshold to promote upper airway patency). For example, the stimulation may cause activation of the SHM 254 alone (e.g., the SHM inferior) and not cause movement of the thyroid cartilage inferiorly. In various examples, the method may further comprise verifying application of stimulation at the second location causes activation of the at least one IHM.

In some examples, the target location is associated with a first side of a body of the patient, and the method further comprises identifying a second target location associated with an opposite second side of the body of the patient for stimulating the IHM-innervating nerve(s) and/or IHM(s), such as the left and right sides of the patient.

After the verification, the method may comprise implanting at least portion of the at least one stimulation element 210 at or near (e.g., in stimulating relation) the target location of the IHM-innervating nerve 215 (and/or IHM), such as for SDB treatment for the patient. In some examples, the at least portion of the at least one stimulation element 210 may comprise a chronically implantable element such as (but not limited to) an electrode cuff to at least partially enclose at least a portion of the IHM-innervating nerve 215 and/or at least one IHM at the target location or such as an axial electrode array or other electrode carrier configurations. In some examples, the stimulation element 210 may be anchored to non-nerve tissue at or near the target location of the IHM-innervating nerve 215 and/or at least one IHM. In some examples, the stimulation element 210 may comprise a stimulation lead, on which at least one stimulation electrode of the least one stimulation element 210 is supported, in stimulating relation to the target location of the IHM-innervating nerve 215 and/or at least one IHM. In some such examples, the stimulation element 210 may comprise a pulse generator, with at least some components implantable and/or at least some components external. In examples in which the stimulation element 210 may comprise at least some implantable components of a pulse generator, such implantable component may support a stimulation electrode on a housing of such components and/or may support removable connection of the above-described lead to a housing of the implantable pulse generator components. Examples of deployment of IMDs are illustrated further herein at least in connection with FIGS. 4-9 and FIGS. 18A-22B.

FIG. 3 is a flow diagram of another example method for identifying a target location of an IHM-innervating nerve. In some examples, the method 330 illustrated by FIG. 3 may comprise part of, and/or is an example implementation of, the method 10 illustrated by FIG. 1A and/or the method illustrated by FIGS. 2A-2G.

As shown at 331 in FIG. 3, in some examples, the method 330 comprises making an incision at about two centimeters to about three centimeters superior to the clavicle and at a level that is approximate to the OHM. The method 330 comprises identifying the OHM, at 333, and retracting the OHM superiorly, at 335. At 337, the method 330 comprises dissecting deep to locate a target nerve branch of the IHM-innervating nerve from a lateral aspect, such as target IHM-innervating nerve branch 242 illustrated by at least FIGS. 1D and 2E. And, at 339, the method 330 comprises implanting at least a portion of at least one stimulation element at or near a first location of the IHM-innervating nerve on the target nerve branch. At 341, the method 330 comprises stimulating at the first location of the IHM-innervating nerve, and at 343, a determination is made on whether the stimulation captures the target at least one IHM (e.g., infrahyoid strap muscle) and/or target physiological response, such as movement of the thyroid cartilage inferiorly. In response to verifying capture of the target IHM and/or target physiological response, at 345, the first location is identified as the target location. In response to not verifying capture of the target IHM and/or target physiological response, at 347, the method 330 comprises moving the at least portion of the at least one stimulation element to a second location (e.g., a revised target location) of the IHM-innervating nerve and repeating the steps of 341, 343, and one of 345 or 347 until a target location is identified. In some examples, the above steps involving stimulating, assessing, and re-stimulating may be implemented using a temporary stimulation test tool. For example, in some examples, a stimulation test tool and/or delivery tool (including stimulation testing features) may be used to identify the stimulation target. In some such examples, identifying the stimulation target may be performed via a minimally invasive technique (percutaneous delivery from incision, intravascular delivery with transvenous stimulation testing, etc.).

In some examples, the IHM-related tissue may alternatively include at least one IHM. The method 330 may be applied to the at least one IHM, such as by stimulating at a first location of the at least IHM and verifying the target physiological response occurs. In response to the physiological response not occurring, the stimulation element may be moved to a stimulate at a second target location of the at least one IHM.

In various examples, the methods and/or variations described in connection with FIGS. 1A-3 may be implemented using an IMD or may be used to implant an IMD. For example, at least one stimulation element (e.g., forming at least a portion of an IMD) may be implanted based on the identified target location of the IHM-innervating nerve. FIGS. 4-5E illustrate different example arrangements of IMDs, including stimulation elements (e.g., pulse generators, leads, electrodes, etc.) and related components.

FIG. 4 is a block diagram schematically representing an example IMD. The IMD 480 may include a stimulation element. The stimulation element may include an IPG assembly 481 and at least one stimulation lead 455. The IPG assembly 481 may include a housing 483 containing circuitry 486 and a power source 488 (e.g., battery), and an interface block or header-connector 484 carried or formed by the housing 483. The housing 483 is configured to render the IPG assembly 481 appropriate for implantation into a human body, and may incorporate biocompatible materials and hermetic seal(s). The circuitry 486 may be implemented, at least in part, via a control portion (and related functions, portions, elements, engines, parameters, etc.) such as described later in connection with at least FIGS. 27A-29.

In some examples, the stimulation lead 455 includes a lead body 450 with a distally located stimulation electrode arrangement 410. At an opposite end of the lead body 450, the stimulation lead 455 includes a proximally located plug-in connector 482 which is configured to be removably connectable to the interface block 484. For example, the interface block 484 may include or provide a stimulation port sized and shaped to receive the plug-in connector 482.

In general terms, the stimulation electrode arrangement 410 may optionally be an electrode cuff, and may include some non-conductive structures biased to (or otherwise configurable to) releasable secure electrically conductive electrodes of the stimulation electrode arrangement 410 about a target nerve. Other formats are also acceptable. In some examples, the stimulation electrodes (such as those having a stimulation electrode arrangement 410) may comprise electrodes to deliver a stimulation signal to a target nerve. Examples are not limited to cuffs and may include stimulation elements having a stimulation electrode arrangement 410 in different types of configurations and/or for different targets, such as an alternating current target, a paddle, and an axial arrangement, among others. The stimulation electrode arrangement(s) may contact a target tissue and/or otherwise be in stimulating relation to the target tissue in a non-contact manner.

In some examples, the lead body 450 is a generally flexible elongate member having sufficient resilience to enable advancing and maneuvering the lead body 450 subcutaneously to place the stimulation electrode arrangement 410 at a desired location adjacent target tissue, such as an upper airway patency-related nerve (e.g., IHM-innervating nerve, hypoglossal nerve) or muscle (e.g., IHM). In some examples, such as in the case of OSA, the nerves may include (but are not limited to) the nerve and associated muscles responsible for causing movement of the tongue and related musculature to restore airway patency. In some examples, the nerves may include (but are not limited to) at least one IHM-32 innervating nerve and the muscles may include (but are not limited to) at least one IHM. In some examples, lead body 450 may have a length sufficient to extend from the IPG assembly 481 implanted in one body location (e.g., pectoral) and to the target stimulation location (e.g., head, neck). Upon generation via the circuitry 486, a stimulation signal is selectively transmitted to the interface block 484 for delivery via the stimulation lead 455 to the nerve.

It will be understood that the interface block 484 is representative of many different kinds and styles of electrical (and mechanical) connection between the housing 483 of the IPG assembly 481 and the lead 455 with such connections having a size, shape, location, etc. which may differ from the interface block 484 shown in FIG. 4.

In some examples, the IMD 480 further comprises at least one implantable sensor 485. The at least one implantable sensor 485 may be connected to the IMD 480 in various fashions, such as being coupled to the interface block 484, being carried by (or within) the IPG assembly 481, and/or wirelessly communicating with the IPG assembly 481. More specifically, the at least one implantable sensor 485 may be connected in various orientations as described within U.S. Patent Publication No. 2021/0268279, published on Sep. 2, 2021, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety, including the determining or designating a posture of the patient based on data from the acceleration sensor. Although the above examples describe an IMD 480 having a stimulation lead 455, examples are not so limited and example IMDs may additionally or alternatively include a lead used for sensing.

It will be understood that the example IMDs in FIGS. 4 and 5A-5E are not limited to the example sensors described in association with FIGS. 4 and 5A-5E but may comprise at least one of the different sensor modalities, placements, etc.

In some examples, the at least one implantable sensor 485 may be wirelessly connected to the IPG assembly 481. In such examples, the interface block 484 need not provide a sense port for the at least one implantable sensor 485 or the sense port may be used for a second sensor (not shown). In some examples, the circuitry 486 of the IPG assembly 481 and circuitry of the at least one implantable sensor 485 communicate via a wireless communication pathway according to known wireless protocols, such as Bluetooth, near-field communication (NFC), Medical Implant Communication Service (MICS), 802.11, etc. with each of the circuitry 486 and the at least one implantable sensor 485 including corresponding components for implementing the wireless communication pathway. In some examples, a similar wireless pathway is implemented to communicate with devices external to the patient's body for at least partially controlling the at least one implantable sensor 485 and/or the IPG assembly 481, to communicate with other devices (e.g., other sensors) internally within the patient's body, or to communicate with other sensors external to the patient's body.

FIGS. 5A-5E are diagrams schematically representing deployment of example stimulation elements. In some examples, the stimulation element may include or form part of an IMD. Example IMDs may be used to stimulate nerves and/or muscles. In some examples, the IMDs illustrated by FIGS. 5A-5E may include an implementation of, and/or include at least some of substantially the same features and attributes as the IMD 480 of FIG. 4.

More specifically, FIG. 5A is diagram including a front view schematically representing deployment 500 of an example IMD 522 including at least one stimulation element. In some examples, the stimulation element comprises an IPG 533, which includes at least one sensor 525, and a stimulation electrode arrangement 512. As shown in FIG. 5A, in some examples, the IPG 533 may be chronically implanted in a pectoral region 513 of a patient and the stimulation electrode arrangement 512 of the stimulation element may be chronically implanted in or near a head-and-neck region 505 of the patient. The at least one implantable sensor 525 may sense data indicative of various physiologic phenomenon sensed from this implanted position (e.g., body motion, posture, vibrations, such as anatomy vibrations and device vibrations). In some examples, the IMD 522 may comprise the IPG 533, such as for managing sensing and/or stimulation therapy. The at least one stimulation electrode arrangement 512 may be implanted at or near the IHM-innervating nerve 515, such as illustrated by the target location which may be identified according to method 10 of FIGS. 1A-1E and/or as described in connection with FIG. 2A-2G in various examples. The stimulation electrode arrangement 512 may include an implementation of, and/or including at least some of substantially the same elements and features as, the example stimulation elements previously described in connection with at least FIGS. 1A-4. The common elements and features are not repeated for ease of reference. Among other features, it will be understood that in some examples a body of a lead 455 (FIG. 4) supports the stimulation electrode arrangement 512, while extending between the IPG 533 and the stimulation electrode arrangement 512. Moreover, in some examples, the IPG 533 may be formed on a smaller scale (e.g., microstimulator) and/or different shape to be amenable for implantation in the head-and-neck region 505 instead of pectoral region 513.

FIG. 5B is a diagram including a front view schematically representing deployment 501 of an example IMD 523 which includes stimulation element comprises an IPG 533 and at least one stimulation electrode arrangement 512. In some examples, IMD 523 includes an implementation of, and/or at least some of substantially the same features and attributes as, the IMD 522 as previously described in connection with at least FIG. 5B, and the IPG 533 may be implanted in a pectoral region 513 and/or include a sensor, as previously described. The common elements and features are not repeated for ease of reference.

In some examples, the IMD 523 comprises a lead 572 including a lead body 578 (e.g., 450/455 in FIG. 4) for chronic implantation (e.g., subcutaneously via tunneling or other techniques) and to extend from the IPG 533 to a position adjacent to the at least one nerve 515 and with at least one stimulation electrode arrangement 512 on an opposite end of the IPG 533. The stimulation electrode arrangement 512 may engage the nerve 515 in a head-and-neck region 505 for stimulating the nerve 515 to treat a physiologic condition, such as SDB.

In some such examples, multiple nerves may be targeted for stimulation, such as illustrated by FIGS. 5D-5E and separate stimulation leads may be provided or a single stimulation lead may be provided but with a bifurcated distal portion with each separate distal portion extending to a respective one of the multiple nerves. For example, and referring to FIG. 5E, a stimulation lead, on which the least one stimulation electrode arrangement 512A, 512B is supported, may be implanted in a position extending between the IPG 533 and a stimulating relation to the at least one nerve 515, 516. Further details regarding applying stimulation to multiple target tissues is described later in association with at least FIGS. 5D-5E.

FIG. 5C is a diagram including a front view schematically representing deployment 503 of an IMD 519A comprising at least some of substantially the same features and attributes as the IMD 523 in FIG. 5B, except with the stimulation element (including IPG 533) implemented as a microstimulator 519B. In some examples, the microstimulator 519B may be chronically implanted (e.g., percutaneously, subcutaneously, transvenously, etc.) in a head-and-neck region 505 as shown in FIG. 5C, or in a pectoral region 513. In some examples, as part of the IMD 519A, the microstimulator 519B may be in wired or wireless communication with stimulation electrode arrangement 512. In some examples, the microstimulator 519B may form part of the stimulation electrode arrangement 512. In some examples, as part of the IMD 519A, the microstimulator 519B may incorporate sensor or be in wireless or wired communication with a sensor located separately from a body of the microstimulator 519B or implanted in the body, as illustrated by sensor 525. When wireless communication is employed for sensing and/or stimulation, the microstimulator 519B may be referred to as leadless IMD for purposes of sensing and/or stimulation. In some examples, the microstimulator 519B may be in close proximity to a target nerve 515.

In some examples, the microstimulator 519B (and associated elements) may comprise at least some of substantially the same features and attributes as described and illustrated in U.S. Patent Publication No. 2020/0254249, published on Aug. 13, 2020, and entitled “MICROSTIMULATION SLEEP DISORDERED BREATHING (SDB) THERAPY DEVICE”, the entire teachings of which is incorporated herein by reference in its entirety.

While FIGS. 5A-5C illustrate a single simulation element and/or stimulation electrode arrangement, in some examples, multiple stimulation elements and/or stimulation electrode arrangements may be implanted in the patient. For example, and as shown in FIGS. 5D-5E, the at least one stimulation element comprises a first stimulation electrode arrangement 512A and a second stimulation electrode arrangement 512B, or more. In some such examples, applying the stimulation comprises applying the stimulation on both a first portion and a second portion, e.g., a right side and opposite left side of FIG. 5D or different areas of the head-and-neck region 505 of a body of the patient via the first stimulation electrode arrangement 512A and the second stimulation electrode arrangement 512B.

FIG. 5D is a diagram including a front view schematically representing deployment 506 of an IMD 524 including stimulation element comprising an IPG 533 and at least two stimulation electrode arrangements 512A, 512B. The stimulation electrode arrangements 512A, 512B may each include an implementation of, and/or at least some of substantially the same elements and features as, the stimulation element and stimulation electrode arrangements previously described in connection with the examples of at least FIGS. 1-5C. Further, the IPG 533 may include an implementation of, and/or at least some of substantially the same elements and features as, the IPG as previously described in connection with the examples of at least FIGS. 1-5C.

As shown by FIG. 5D, each stimulation electrode arrangement 512A, 512B may be chronically implanted in or near a head-and-neck region 505 of the patient, with the first stimulation electrode arrangement 512A being implanted on a right side of the patient and the second stimulation electrode arrangement 512B being implanted on an opposite left side of the patient. Each stimulation electrode arrangement 512A, 512B may be implanted at or near for coupling to the IHM-innervating nerves 515A, 515B on the right and left sides of the head-and-neck region 505 of the patient, such as illustrated by the target location T illustrated at least by FIGS. 1D and 2E.

In some examples, additional stimulation elements and/or stimulation electrode arrangements may be implanted, such as to target multiple target locations of the IHM-innervating nerves 515A, 515B and/or to target other upper airway patency-related tissue, including but not limited to the stylopharyngeus muscle, the hypoglossal nerve, the genioglossus muscle, IHMs, among other tissue.

FIG. 5E is a diagram schematically representing an example deployment 507 of an IMD 526 comprising at least some of substantially the same features and attributes as the IMD 523 in FIG. 5B and the IMD 519A in FIG. 5C, such that the IMD 526 includes a stimulation element comprising both an IPG 533 implanted in a pectoral region 513 and a microstimulator 519B implanted in the head-and-neck region 505. In some examples, the IMD 526 of FIG. 5E includes an implementation of, and/or at least some of substantially the same features and attributes, as the IMDs of any of FIGS. 5A-5D. The common features and attributes are not repeated.

As shown by FIG. 5E, the IMD 526 comprises multiple stimulation electrode arrangements 512A, 512B for chronic implantation (e.g., subcutaneously via tunneling or other techniques) at a position adjacent a nerve (e.g., IHM-innervating nerve 515 and hypoglossal nerve 516). The stimulation electrode arrangements 512A, 512B may comprise electrodes to engage the nerves, e.g., 515, 516 in a head-and-neck region 505 for stimulating the nerve(s) to treat a physiologic condition, such as SDB. The IMD 526 (and any of the IMDs illustrated by FIGS. 5A-5E) may comprise a control portion (e.g., circuitry, power element, etc.) to support control the stimulation electrode arrangements 512A, 512B (via lead(s) 572 or wirelessly) and other component, such as at least one sensor. In some examples, such control, operation, etc., may be implemented, at least in part, via a control portion 2100 (and related functions, portions, elements, engines, parameters, etc.) such as described later in connection with at least FIGS. 27A-29.

In some examples, any of the devices of FIGS. 5A-5E may be deployed to stimulate at least one IHM, such as further illustrated by example stimulation elements described further herein in connection with FIGS. 18A-22B.

FIGS. 6A-6C are diagrams schematically representing patient anatomy and an example device and/or example method for identifying a target location for stimulating an IHM-related tissue, such as an IHM-innervating nerve.

FIG. 6A is a diagram including a side view schematically representing an example arrangement 3000 including a stimulation element comprising a stimulation electrode arrangement 212 in stimulating relation to the IHM-innervating nerve 215 (e.g., at branch 242) and a supporting stimulation lead 2917 anchored relative to non-nerve structure 2929 (e.g., tissue). In some examples, the stimulation element including the stimulation electrode arrangement 212 and/or stimulation lead 2917 may comprise an example implementation of, and/or at least some of substantially the same features and attributes as stimulation elements (and related arrangements) described in association with various examples described in association with at least FIGS. 1A-5E. FIG. 6A further illustrates the anatomy as previously described in connection with FIGS. 1A-1E and FIGS. 2A-2G, as shown by the common numbering. The common features and attributes are not repeated for ease of reference.

It will be understood that the particular location of the stimulation electrode arrangement 212 (e.g., at least one electrode) and the anchor location in FIG. 6A is merely representative of many different target potions and anchor locations at which the stimulation electrode arrangement 212 may be located. For example, in some examples, the stimulation element arrangement 212 may be in stimulating relation to at least one IHM, such as the STM 244 and/or SHM 254, whether such stimulation is an alternative to stimulating the IHM-innervating nerve or in addition to stimulating an IHM-innervating nerve. In some such examples, stimulating both the IHM-innervating nerve and the IHM may comprise stimulating a neuromuscular junction (e.g., end motor point) of such nerves and muscles.

As shown in FIG. 6A, the stimulation lead 2917 includes a distal portion 2919 which may be formed into a strain relief loop or portion extending between the stimulation electrode arrangement 212 and the fixation arrangement 2927, with the fixation arrangement 2927 secured to the non-nerve structure 2929 in order to secure the stimulation lead 2917 thereto. A lead body 2921 of the stimulation lead 2917 may extend proximally from a portion of the fixation arrangement 2927.

As further shown in FIG. 6B, box 2950 schematically represents at least some of the non-nerve structures 2929 (in FIG. 6A) to which the fixation arrangement 2927 may anchor a portion of the stimulation lead 2917. In some examples, such non-nerve structures may comprise an omohyoid tendon, a hyoid bone, a clavicle, a sternum (including the manubrium), a trachea, a digastric tendon, and/or other non-nerve structures. Moreover, such non-nerve structures may be used for anchoring a stimulation lead, port interface (e.g., FIGS. 5A-5E, and the like), stimulation element, etc. relative to an upper airway patency-related tissue, whether in relation to the example of FIGS. 6A, 6C and/or other examples.

FIG. 6C is a diagram including a side view schematically representing an example arrangement 3100 which comprises a stimulation element that comprises an implementation of, and/or at least some of substantially the same features and attributes as, the example arrangement 3000 in FIGS. 6A-6B, except with a distal portion of a stimulation lead 3117 including a pre-formed strain relief segment 3119 between the fixation arrangement 2927 and the stimulation electrode arrangement 212. The pre-formed strain relief segment 3119, shown within the dashed lines, may comprise any flexible, resilient shape (e.g., sigmoid, other) which helps to relieve strain on the stimulation electrode arrangement 212 in its fixed position relative to a nerve or muscle to be stimulated, such as strain occurring during movement of the neck and/or other body movements.

It will be understood that the fixation arrangements (e.g., fixation elements, non-nerve structures, strain relief segments, etc.) described in association with at least FIGS. 6A-6C may be implemented in various forms with any of the stimulation elements, electrode arrangements, stimulation leads, port interfaces, sensing leads, etc. as described throughout the various examples of the present disclosure.

As implicated by the above description, the IMDs may include a controller, control unit, or control portion that prompts, controls, tracks, etc., performance of designated actions.

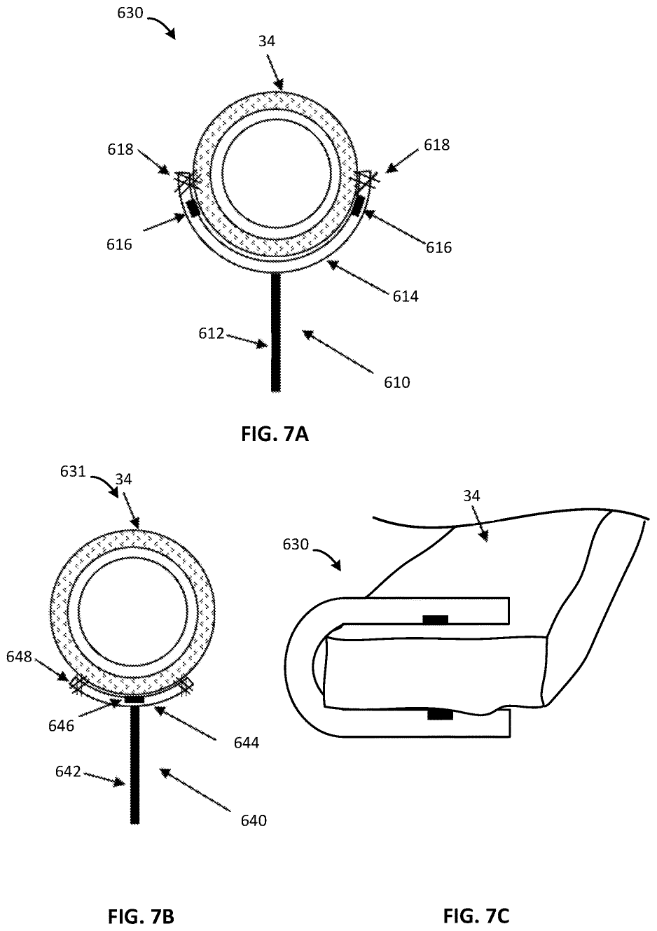

FIGS. 7A-17EG are diagrams representing example stimulation elements. Any of the stimulation elements illustrated in connection with FIGS. 7A-17EG may be an example implementation of (at least some of the features of) the stimulation elements of FIGS. 1F-1G or FIGS. 4-6C, and/or may be used to implement the method 10 illustrated by FIG. 1A and/or the method illustrated by FIGS. 2A-2G.

FIG. 7A illustrates an example stimulation element 630 including a lead 610 having a lead body 612 and a head 614 carrying stimulation electrodes 616. The head 114, which may be referred to as a stimulation support element, is configured to maintain the electrodes 616 (as well as other optional electrical components) in an electrically isolated manner, and may have the curved or U-shape reflected by the view or other shapes suited to the shape of the particular muscle being engaged. In the example of FIG. 7A, the lead 610 has been delivered within the body of the patient, locating the head 614 about a segment of at least one IHM 34 and/or an IHM-innervating nerve. With this arrangement, the stimulation electrodes 616 are positioned to deliver stimulation energy to the IHM 34 and/or an IHM-innervating nerve adjacent thereto. The head 614 may be secured or anchored relative to the IHM 34 (or IHM-innervating nerve) in various manners, for example via sutures 618. In some examples, the head 614 may have a flexible configuration, allowing a clinician the ability to form or shape the head 614 to a size and/or shape (e.g., curved, flat, etc.) of the at least one IHM 34. In other examples, the head 614 may more rigidly retain a pre-formed shape. Regardless, the lead body 612 may be routed to a stimulation energy source, such as an IPG. While FIGS. 7A-7B illustrate the muscle as being circular, various muscles or portions thereof are non-circular in cross-sectional shape. For example, the example stimulation element 630 of FIG. 7A is shown placed at least partially around muscle tissue of at least one IHM 34 that exhibits or has a non-circular cross-section as shown by FIG. 7C.

Portions of another stimulation element 631 as implanted to a patient in accordance with principles of the present disclosure are shown in FIG. 7B. The stimulation element 631 includes a lead 640 having a lead body 642 and a head 644 carrying a stimulation electrode 646. The head 644 may have the curved or U shape reflected by the view. Similarly, the at least one IHM 34 may have curved shapes, such as non-circular (e.g., oblong) cross-sections. In the example of FIG. 6B, the lead 640 has been delivered to the body, locating the head 644 about a segment of at least one IHM 34. With this arrangement, the stimulation element 646 is positioned to deliver stimulation energy to the at least one IHM 34 and/or an IHM-innervating nerve.

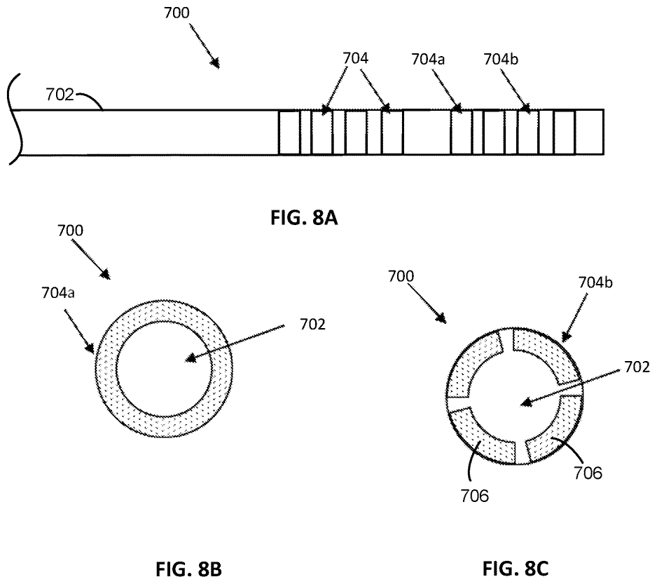

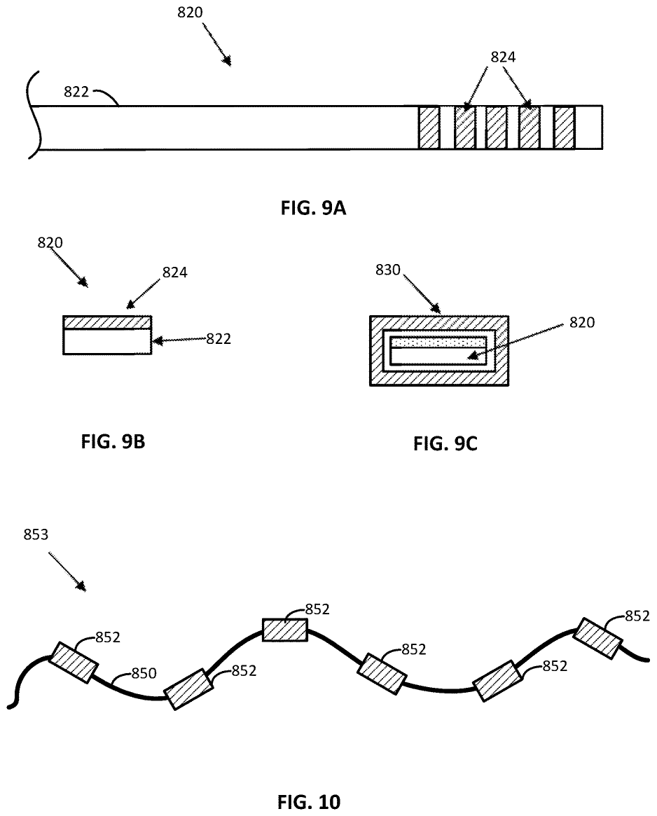

FIGS. 8A-9C illustrate example stimulation elements comprising a lead with an array of stimulation electrodes. In some examples, the stimulation elements may include an axial electrode array of a plurality of electrodes supported by a lead (e.g., an axial arrangement of electrodes). The electrodes of the array may be linear electrodes or ring electrodes, among other configurations. In some examples, the particular arrangement (e.g., number, shape, spacing, orientation, etc.) of electrodes may be different from the particular arrangement of electrodes illustrated by FIGS. 8A-9C.

For example, FIGS. 8A-8C illustrate an example lead 700 which includes a lead body 702 and a plurality of stimulation electrodes 704, which may form a stimulation electrode arrangement. The lead body 702 is configured to maintain the electrodes 704 (as well as other optional electrical components) in an electrically isolated manner, and may have the cylindrical shape as shown by FIGS. 8B-8C. The lead body 702 is formed of a biocompatible material appropriate for implantation into the human body.

Each of the stimulation electrodes 704 are formed of an electrically conductive material appropriate for delivering stimulation energy within the human body. The stimulation electrodes 704 may assume any of the constructions of the present disclosure. The stimulation electrodes 704 are arranged along the lead body 702 to provide an exposed surface from which stimulation energy is emitted. The stimulation electrodes 704 are electrically isolated from one another by the lead body 702 and non-exposed portions of the electrodes 704 are encapsulated by the lead body 702.

In some examples and as best reflected by FIG. 8B, at least one of the stimulation electrodes, for example the stimulation electrode 704a, may be a complete ring-type electrode. As reflected by FIG. 8C, at least one or all of the stimulation electrodes, for example the stimulation electrode 704b, may be, or may be akin to, a split ring-type electrode, comprised of two or more electrode segments 706. Individual, electrically isolated wire(s) may extend from each of the stimulation electrode segments 706 within a thickness of the lead body 702. With these and related examples, the electrode segments 706 are individually selectable and provide anti-rotation attributes. The selectable nature of the electrode segments 706 allows for the ability to include or exclude various tissue (e.g., nerves, muscles) upon final implant/use. The segmented leads of the present disclosure, such as the lead 700, may include or carry a fixation arrangement (e.g., ridge, tines, fins, frictional tissue-engaging portions, etc.) that maintain both axial and rotational stability of lead 700.

Another lead 820 in accordance with the present disclosure, is shown in FIGS. 9A-9C. The lead 820 may include a lead body 822 and a plurality of stimulation electrodes 824, which together may form a stimulation electrode arrangement. The lead body 822 is configured to maintain the electrodes 824 (as well as other optional electrical components) in an electrically isolated manner. The lead body 822 is formed of a biocompatible material appropriate for implantation into the human body.

The lead body 822 has a non-circular shape in transverse cross-section, as shown in FIG. 9B. With the non-limiting example, the electrodes 824 may be placed on one side of the lead body 822. Upon final implant, an electrical field generated by the electrodes 824 may be preferentially directed. For example, by optionally arranging all of the electrodes 824 on a single side of the lead body 822, the electrical field generated by the electrodes 824 may be preferentially directed at a particular IHM (e.g., STM) or an IHM-innervating nerve or other target nerves while excluding tissue/structures located at the opposite side of the lead body 822. In some examples, the non-circular leads of the present disclosure, such as the lead 820, are well-suited for introduction into the patient in a desired orientation via a non-circular introducer, such as the introducer 830 shown in FIG. 9C.

In some examples, such as at least the example in FIG. 10, the stimulation element may comprise a more flexible, resilient structure, which functions in part, as a retention element for robustly securing the portions of the stimulation to tissue. In some such examples, the body of the lead and/or carrier (in the region supporting the electrodes of the stimulation element) may be pre-formed in a shape, size, and/or orientation adapted to promote anchoring or fixation of the stimulation electrodes relative to the pertinent anatomical features in which the stimulation element is to become secured. In some examples, the pre-formed shape may be implemented via a shape memory material. Via such arrangements, because of its flexible resilience, such a stimulation element may be manipulated from its original shape in order to introduce and advance the stimulation electrodes along a delivery path, with the stimulation element being biased to return as close as possible to its original shape, which in turn helps to secure the stimulation element in a desired location. In some examples, these above-noted size, shape, and/or orientation features may be implemented in the example stimulation element 853 of FIG. 10.

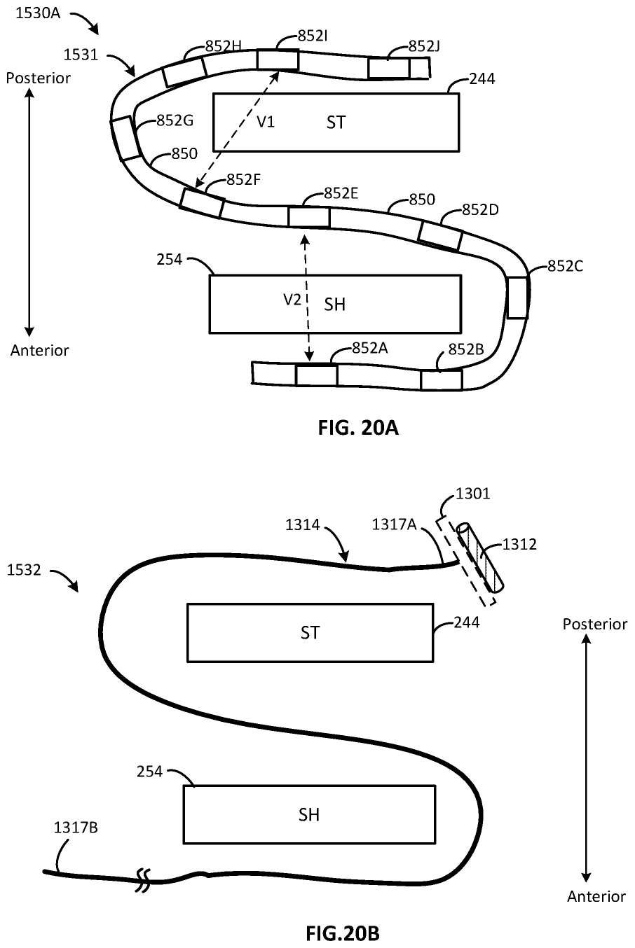

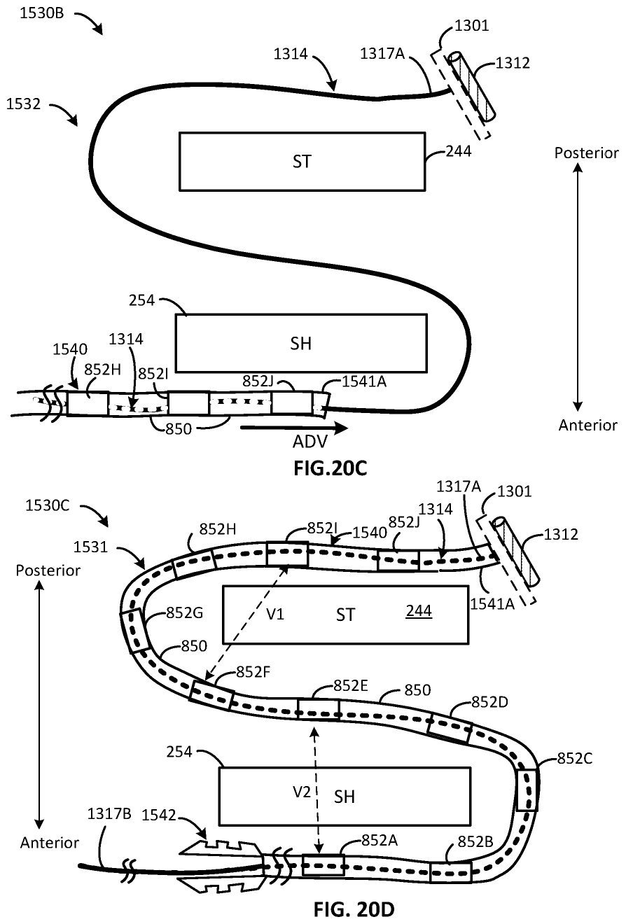

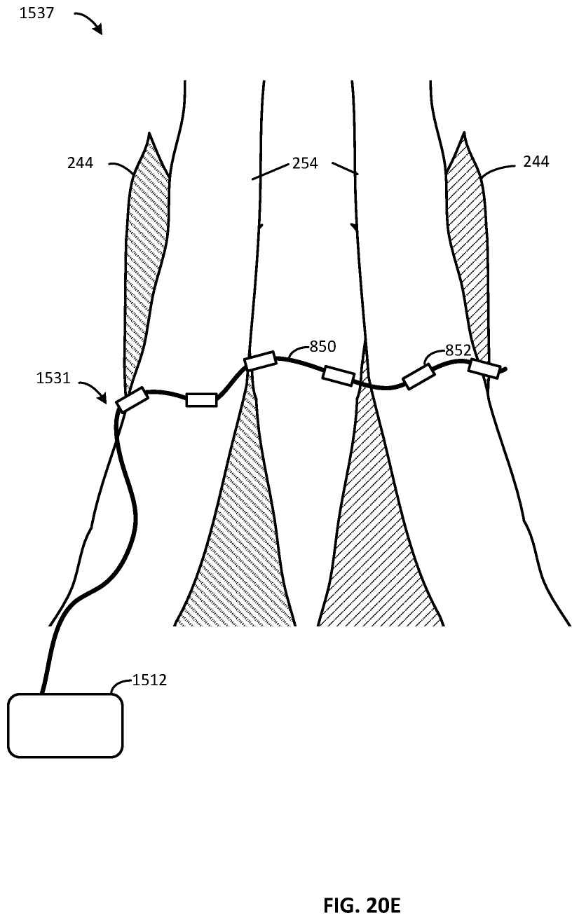

FIG. 10 illustrates an example stimulation element 853 comprising a lead portion 850 that supports an array of spaced apart stimulation electrodes 852. The lead portion 850 may comprise a distal portion of the lead which extends from a main lead portion that is more proximal to the distal portion. Each lead portion 850, including the main lead portion, may be formed of a flexible, resilient material to implement functions of an implantable medical lead, with the distal lead portion 850 carrying the electrodes 852 and which may have a higher degree of flexibility and/or degree of configurability, while still retaining their resilience (e.g., biased to maintain and/or return to shape), in order to permit the electrodes 852 and distal lead portion 850 to be manipulated into a position and shape within the body to help secure the stimulation electrodes 852 as desired. In some examples, the entire lead may be the more flexible form. It may be appreciated that the electrodes 852 may embody different example shapes while still providing the features and attributes of the example, and the rectangular shape and size are merely an illustrated example. In some examples, all of the separate electrodes 852 may be in stimulating relation to the same general target tissue, such as a single muscle (or nerve). However, in some examples, at least some the separate electrodes 852 are in stimulating relation to one target tissue (a muscle or nerve) while other of the electrodes are in stimulating relation to a different target tissue (muscle) such as some on STM and some on SHM, such as further illustrated in connection with at least FIG. 20A.

In some examples, the stimulation element 853 (or any of the stimulation elements illustrated herein) may comprise a fixation arrangement (including fixation element(s)) mounted or formed at an utmost distal end of the stimulation element 853. It will be understood that, in some examples, at least some features of the fixation arrangement may be implemented at locations along the lead other than the utmost distal end, such as at an opposite proximal end of the distal lead portion 850 or other portions of the lead body.

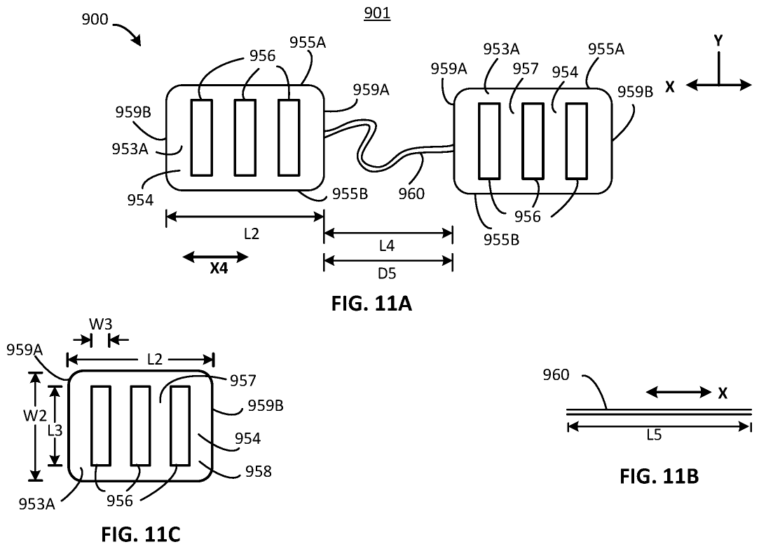

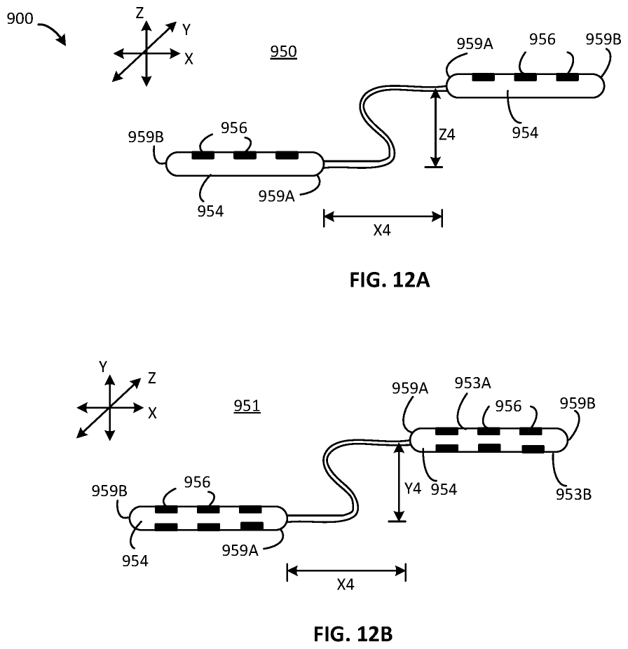

FIGS. 11A-12B illustrate example stimulation elements comprising a paddle-style body 954 supporting at least one stimulation electrode 956. As shown by at least FIG. 11A, the stimulation element 900 may comprise a pair of paddle-style bodies 954 connected by a connector segment 960. The pair of paddle-style bodies 954 may be independently positionable.

Each paddle-style body 954 supports at least one stimulation electrode, such as an array of stimulation electrodes 956 which are in a spaced apart relationship with electrically non-conductive portions 957 of the body 954 on a first surface 953A interposed between adjacent pairs of stimulation electrodes 956. The body 954 includes an opposite second surface (953B in FIG. 12B and with portions 956, 957 being on first surface 953A in FIG. 11C). In some examples, the second surface 953B is electrically non-conductive. In some examples, as illustrated by FIG. 12B, the second surface 953B additionally includes an array of stimulation electrodes 956 spaced apart by electrically non-conductive portions 957 of the body 954. In some examples, the first surface 953A may face toward target tissue (e.g., nerves or muscle). In some examples, the target tissue may be behind (e.g., posterior or deeper) the body of each paddle-style body 954. The stimulation electrodes 956 may be spaced apart by the non-conductive portions 957 to be independently controlled and to independently apply stimulation signals, in some examples.

In some examples, the stimulation element 900 further comprises a lead which connects to the pair of paddle-style bodies 954 (e.g., at ends 959A, which are opposite the outer or distal ends 959B of the bodies 954). In some examples, a distal portion of the lead may comprise the flexible connector segment 960 which extends between and at least mechanically connects the paddle-style bodies 954 relative to each other. In some examples, each paddle-style body 954 of the stimulation element 900 is electrically connected via lead body to a pulse generator.

The flexible connector segment 960 (sometimes referred to as a bifurcation portion) may form a variety of shapes, such as being T-shaped, Y-shaped, or linear. In some examples, the flexible connector segment 960 may comprise a single or a plurality of independent electrical conductors with each such independent electrical conductor establishing electrical connection between a respective one of the stimulation electrodes 956 and, optionally, with corresponding independent electrical conductors within proximal portions of lead body, which in turn are in electrical connection with electrical contact portions of a port and/or of a pulse generator which delivers electrical stimulation signals to the stimulation electrodes 956. In this regard, in some such examples, the flexible connector segment 960 omits stimulation generation circuitry, omits wireless power-receiving circuitry, and/or omits wireless communication circuitry. In some examples, the flexible connector segment 960 may have a generally cylindrical shape, such that has a generally circular cross-sectional shape.

In some examples, the flexible connector segment 960 generally does not perform functions other than transmitting stimulation signals from a pulse generator/microstimulator to the stimulation electrodes 956. In some such examples, the electrical conductor(s) extending within and through the flexible connector segment 960 generally comprise the sole electrically conductive elements within the flexible connector segment 960.

In general terms, the connector segment 960 is flexible to permit independent positioning of each respective paddle-style body 954 relative to target tissues, such as nerves, muscles, combinations of nerves and muscles, neuromuscular junctions (e.g., nerve endings) of nerves and muscles, and/or combinations thereof. In some examples, the flexible connector segment 960 comprises a flexible material which is selectively bendable into a desired shape, orientation, etc., and which may be maintained in the achieved shape, orientation, etc. with the support of fixation elements used to help maintain the shape, orientation relative to the surrounding tissues so that each respective paddle-style body 954 is retained in a fixed position of stimulating relation to target tissue.

In some examples, the flexible connector segment 960 comprises a material which is selectively manipulable (e.g., bendable, rotatable, etc.) into a desired shape, orientation, etc., and which is made of a material which may retain the selectively manipulated shape, orientation, etc. in order to cause each respective paddle-style body 954 to be retained in its chronically implanted position having a desired orientation, position, etc., of stimulating relation to target tissues.

In some examples, the stimulation element including the paddle-style bodies 954 and flexible connector segment 960 may be implemented to include at least some of substantially the same features and attributes as described in PCT Publication No. WO 2023/150158, published on Aug. 10, 2023, and incorporated above.

In some examples, the paddle-style bodies 954 implement a translational degree of freedom for either one (or both) paddle-style bodies 954. FIGS. 11A-12B are a series of diagrams schematically representing movement of the paddle-style bodies 954 in translational orientation relative to each other. Each of FIGS. 11A, 12A, and 12B schematically represent an example device (and/or example method) including a paddle style body 954.

As shown in the diagram 901 of FIG. 11A, in some examples, when in a relaxed configuration the flexible connector segment 960 of stimulation element 900 exhibits a nominal effective length L4 extending between the respective paddle-style bodes 954. It will be understood that the nominal effective length L4 may vary depending on the number of curves, bends, etc. which may occur in a random manner along the flexible connector segment 960, which corresponds depends on a distance D5 between the paddle-style bodies 954 (e.g., ends 959A (and/or side edges 955A, 955B)). Conversely, as shown in FIG. 11B, when in a fully extended configuration, the flexible connector segment 960 exhibits a maximum length L5 between the respective stimulation elements paddle-style bodies 954, which is greater than the relaxed length L4 in FIG. 11A. In some examples, the maximum length L5 may correspond generally to a length L2 of the body 954 of each respective paddle-style bodies 954.

As further represented in the diagram 900 of FIGS. 11A-11B, in some examples a change in distance between the respective paddle-style bodies 954 corresponds to translational movement along an x orientation (e.g., axis) as represented by directional arrow X4 and which corresponds to one translational degree of freedom. Such translation according to one reference orientation (X) may be implemented with or without rotational movement of the respective paddle-style bodies 954 relative to each other according to one or a combination of the roll parameter, yaw parameter, and a pitch parameter. Such translation according to one reference orientation (X) may be implemented with or without translational movement of the respective paddle-style bodies 954 relative to each other according to the other translational orientations (e.g., Z).

As further represented in the diagram 950 of FIG. 12A, in some examples the paddle-style bodies 954 also may be translated according to a Z reference orientation (as represented by directional arrow Z4), and which corresponds to one translational degree of freedom. Such translation may be implemented with or without rotational movement of the respective stimulation paddle-style bodies 954 relative to each other according to one or a combination of the roll parameter, yaw parameter, and a pitch parameter. Such translation according to one reference orientation (Z) may be implemented with or without translational movement of the respective paddle-style bodies 954 relative to each other according to the other translational orientations (e.g., Y).

As further represented in the diagram 951 of FIG. 12B, in some examples the paddle-style bodies 954 also may be translated according to a Y reference orientation (as represented by directional arrow Y4), and which corresponds to one translational degree of freedom. Such translation may be implemented with or without rotational movement of the respective paddle-style bodies 954 relative to each other according to one or a combination of the roll parameter, yaw parameter, and a pitch parameter. Such translation according to one reference orientation (Y) also may be implemented with or without translational movement of the respective paddle-style bodies 954 relative to each other according to the other translational orientations (e.g., Z).

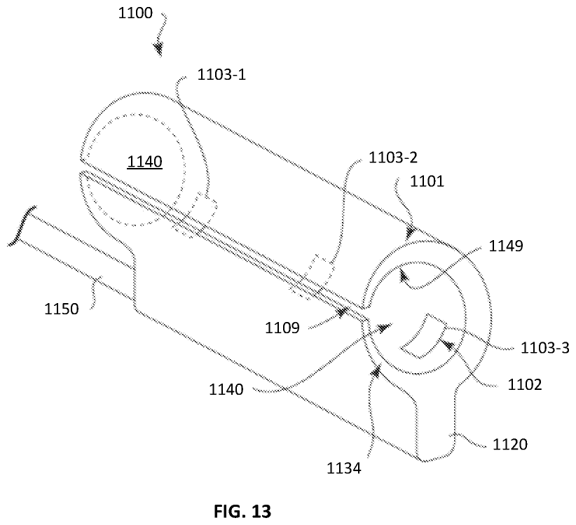

FIG. 13 illustrates an example stimulation element comprising an electrode cuff. More particularly, FIG. 13 illustrates an electrode cuff 1100 comprising a cuff body 1101, which in some examples may be implemented as the stimulation element 110 in FIG. 1F. In some examples, as illustrated by FIG. 13, the electrode cuff 1100 may be coupled to a lead body 1150.

In some examples, the electrode cuff 1100 may comprise a cuff body 1101 and at least one electrode, such as the array 1102 of electrodes 1103-1, 1103-2, 1103-3. In some examples, the cuff body 1101 defines a lumen 1140 through which a target nerve or other body structure may extend. Among other features, the cuff body 1101 may comprise a pair of arms 1134, 1149 (e.g., flange members) that have a generally arcuate shape and that extend from a base 1120 of the cuff body 1101. By pulling the ends of the resiliently, in some examples, biased arms 1134, 1149 apart from each other, access to lumen 1140 is provided for engaging a target nerve and/or muscle. Upon release of the arms 1134, 1149, the cuff body 1101 may resume the shape illustrated in FIG. 13.

In some examples, electrodes 1103-1, 1103-2, 1103-3 are embedded within a wall of the cuff body 301 with the respective electrodes 1103-1, 1103-2, 1103-3 spaced apart from each other along a length of the cuff body 1101. In some examples, the electrodes 1103-1, 1103-2, 1103-3 are aligned in series along a single longitudinal axis on a common side or portion of the cuff body 301. In various examples, the electrode cuff 1100 additionally comprises an outer (third) arm 1170 that is biased and configured to maintain releasable coverage of at least a portion of an outer surface of the cuff body 1101 and of a re-closable opening 1109 between the distal portions of arms 1134, 1149.

In some non-limiting examples, the body 1101 and/or electrode cuff 1100 may comprise at least some of substantially the same features and attributes as described within at least U.S. Patent Publication No. 2011/0160827, published on Jun. 30, 2011 and entitled “ELECTRODE LEAD SYSTEM”, U.S. Pat. No. 9,227,055 issued Jan. 5, 2016 and entitled “SELF EXPANDING ELECTRODE CUFF”, and/or U.S. Pat. No. 8,340,785 issued Dec. 25, 2012 and entitled “SELF EXPANDING ELECTRODE CUFF”, the entire teachings of which are incorporated herein by reference in their entireties.

Any of the above describe stimulation elements may further include or form part of fixation arrangements. In some examples, the fixation arrangements may include fixation elements which are arranged on or otherwise coupled to the stimulation electrode arrangement, lead, or other portions of the stimulation element. FIGS. 14A-17EG illustrate example fixation arrangements. Example fixation arrangements may comprise an array of fixation elements (e.g., tines, barbed elements, etc.) which are flexible and resilient, with such elements sized, shaped, oriented, and/or positioned to engage (e.g., frictionally or otherwise) non-nerve tissues, such as muscle tissue. In some examples, the fixation elements may be oriented to permit forward movement (advancing) of the stimulation element while preventing or hindering movement of the stimulation element in the opposite direction, such as tines located on a distal most end. In some examples, the fixation arrangements may be implemented and/or include at least some of substantially the same features and attributes as described by US Publication 2023/0172479, published on Jun. 8, 2023, and entitled “SINGLE OR MULTIPLE NERVE STIMULATION TO TREAT SLEEP DISORDERED BREATHING”; and/or PCT Publication WO 2023/150158, published on Aug. 10, 2023, and entitled “IMPLANTABLE STIMULATION ELEMENTS AND METHODS FOR SLEEP DISORDERED BREATHING (SDB) CARE”, which are each incorporated herein by reference in their entireties for their teaching.

FIGS. 14A-14B illustrate an example fixation arrangement which comprises a flexible attachment device 1302. The flexible attachment device 1302 includes a tether 1314 and a catch structure 1312. As a point of reference, FIG. 14A illustrates securement of the attachment device 1302 to tissue 1301 at or near the IHM-innervating nerve and/or at least one IHM; other target locations are equally acceptable. At least a distal portion of the tether 1314 is a flexible, high tensile strength body (e.g., suture, permanent braided suture, thread, small diameter wire, etc.). In some examples, the entirety of the tether 1314 is highly flexible. The catch structure 1312 may be a rigid, rod like body (among other body shapes) connected to the tether 1314. An arrangement and configuration of the tether 1314 and the catch structure 1312 is such that in the absence of external forces, the catch structure 1312 may pivot relative to the length of the tether 1314. For example, FIG. 14A illustrates the attachment device 1302 in conjunction with a delivery needle 1310. During use, the needle 1310 is deployed such that a tip 1311 thereof initially contacts a first side 1303 of the tissue 1301, then pierces through the tissue 1301, and is finally located beyond a second side 1304 of the tissue 1301 as shown. The attachment device 1302 is then advanced through a lumen of the needle 1310, deploying the catch structure 1312 from the tip 1311 at a location beyond the second side 1304 of the tissue 1301. In this regard, the flexible nature of the tether 1314 readily facilitates slidable arrangement of the catch structure 1312 within the lumen. By way of further explanation, in the view of FIG. 14A, the catch structure 1312 is oriented such that a major axis of the catch structure 1312 is substantially parallel with an axis of the needle 1310 (and thus the needle lumen); a flexibility of the tether 1314 permits the catch structure 1312 to freely rotate or pivot such that the major axis is aligned with the needle lumen for passage through the needle 1310.

With the catch structure 1312 now located beyond the second side 1304 of the tissue 1301, the needle 1310 may be removed from the patient, leaving the attachment device 1302 in place. As shown in FIG. 14B, the catch structure 1312 remains on the second side 1304 of the tissue 1301 so that when a pulling force is applied onto the tether 1314, the catch structure 1312 is pulled into engagement against second side 1304. With the tether 1314 under tension, a lead of a stimulation element 1305 may be inserted over the tether 1314 and slidably advanced toward the tissue, as further illustrated by FIGS. 20C-20D as an example. The lead may have any of the configurations of the present disclosure, and devices an open central lumen for slidably receiving the tether 1314. Once a desired location of the stimulation element 1305 is achieved, the stimulation element 1305 may be locked to the tether 1314, thereby fixing the stimulation element 1305 relative to the target site. In some examples, as the lead of the stimulation element 1305 is being advanced along the tether 1314, the stimulation element 1305 may be periodically operated to deliver stimulation energy, allowing the clinician to confirm a desired location of the stimulation element 1305. The stimulation element 1305 may be locked onto the tether 1314 in various manners. For example, the stimulation element 1305 (e.g., via a lead) may include or carry a locking feature configured to be crimped or clenched onto the tether 1314. Alternatively or in addition, the tether 1314 may be tied onto the stimulation element 1305. In yet other examples, an adhesive bonding agent may be applied to lock the stimulation element 1305 to the tether 1314.

In some examples, the flexible attachment device 1302 may assume a variety of other forms. For example, in some examples, the tether 1314 may have a multi-component structure, such as a rigid rod proximal section and a small, flexible body distal section (e.g., suture anchor). With these and related examples, the lead of the stimulation element 1305 may more easily slide over the rigid rod proximal section, with the flexible body distal section permitting desired rotation or pivoting of the catch structure 1312 as described above. Further, while the catch structure 1312 has been shown and described as being a rod-like body, other constructions are also envisioned. For example, the catch structure 1312 may have or carry at least one barb that expands after piercing into the tissue 1301. Alternatively or in addition, the catch structure 1312 may include or consist of a mesh-type body that, after deployment, promotes tissue growth, providing a secure attachment point for the tether 1314 over time. Alternatively or in addition, the catch structure 1312 may include or carry a staple or similar bendable structure configured to clinch into tissue. Alternatively or in addition, the catch structure 1312 may include or carry a shape-memory material configured to capture tissue after deployment when it self-reverts to a predetermined shape. Alternatively or in addition, the catch structure 1312 may include or carry a coil that clinches into tissue (e.g., the perineal membrane).

In some examples, the catch structure 1312 and optionally the associated tether 1314 may be substituted for at least some of the fixation arrangements and/or elements throughout the disclosure.

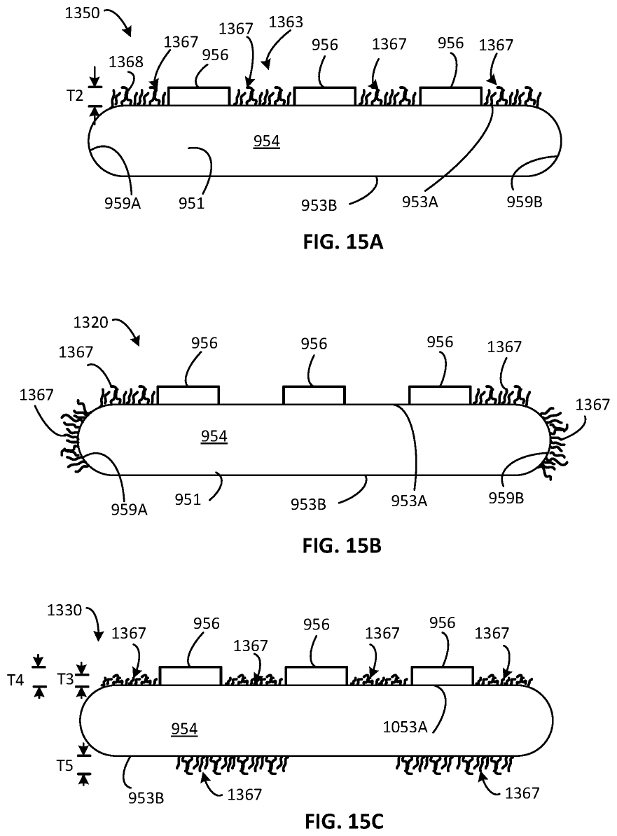

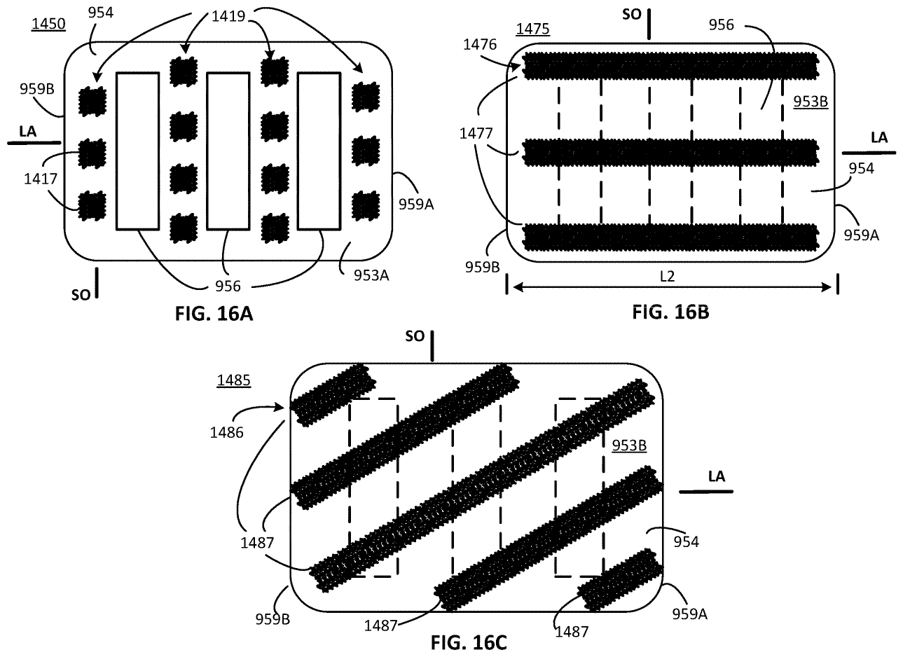

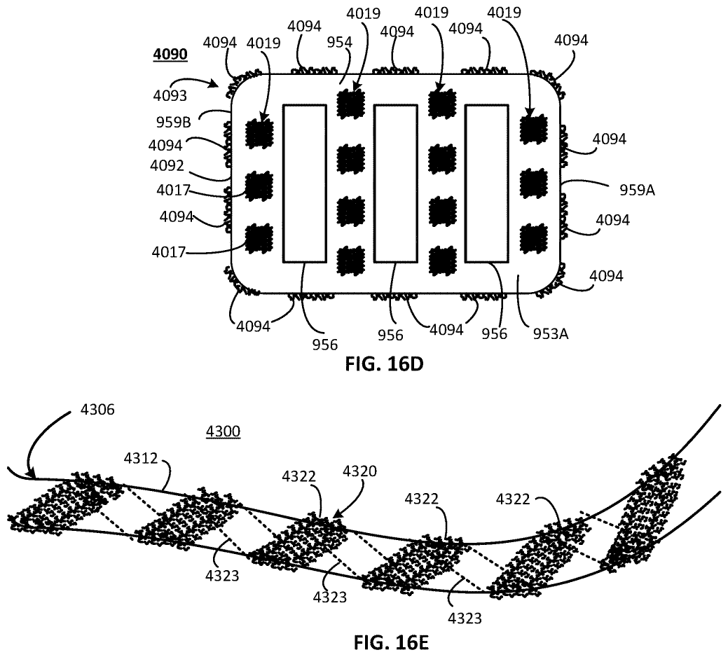

FIGS. 15A-16E show example fixation arrangements on stimulation elements comprising paddle-like bodies 956, such as the paddle-like bodies previously described in connection with FIGS. 11A-12B. The common features are not repeated for ease of reference. In some such examples, the stimulation elements (e.g., stimulation electrode arrangement and/or lead) and fixation elements 1368 may form a fixation arrangement 1363.

As shown in FIG. 15A, in some examples, a fixation arrangement 1363 may comprise a plurality of anchor portions 1367, each of which comprise a plurality of fixation elements 1368. In some examples, each anchor portion 1367 may comprise a plurality of fixation elements 1368 configured to engage surrounding tissue (e.g., target tissue and/or non-target tissue) to secure the stimulation element generally and to secure the stimulation electrodes 956 into stimulating relation to the target tissue such as nerve portions, muscle portions, combinations of nerve portions and muscle portions, neuromuscular junctions of nerve portions and muscle portions, and/or combinations thereof. In some examples which include a stimulation element having a lead, the lead body may include fixation elements 1368 arranged on the lead body. In some examples, the lead body and the stimulation electrode arrangement may include fixation elements 1368.

As shown in the diagram 1350 of FIG. 15A, the various anchor portions 1367 may be located on the stimulation surface 953A of paddle-like body 954 and interposed between adjacent electrodes 956 and in some examples, also may be located on the outer ends of the plurality of electrodes 956, such as shown in FIG. 16B. In this configuration, the anchor portions 1367 act to engage target tissue and/or non-target tissue immediately adjacent to the electrodes 956 to facilitate engagement of the electrodes 956 in stimulating relation to the target tissue.

As shown in the diagram 1320 of FIG. 15B, in some examples, anchor portions 1367 are located on the ends 959A, 959B (and/or side edges) of the body 954 of the stimulation elements but are omitted from the locations between adjacent electrodes 956. In some such examples, this configuration may enhance engagement of the electrodes 956 with the surrounding target tissue and non-target tissue while still providing anchor portions 1367 in close proximity to the electrodes 956. In some examples, this configuration may be desirable in example stimulation elements in which electrodes 956 are flush (or have a low profile) relative to surface 953A because the absence of anchor portions 1367 between electrodes 956 may facilitate more direct engagement of the electrodes 956 with the target tissues.

As shown in the diagram 1330 of FIG. 15C, in some examples anchor portions 1367 may be located on a non-stimulation surface 953B (e.g., a back side) of the body 954 while some anchor portions 1367 may be located on the stimulation surface 953A or omitted from the stimulation surface 953A. The anchor portions 1367 on the non-stimulation surface 953B may enhance securing the body 954 relative to surrounding non-target tissues. For example, upon closing an implant-access incision, anchor portions 1367 on the non-stimulation surface 953B may engage more superficially-located tissue above the body 954, thereby providing additional fixation.

While FIG. 15C shows non-stimulation surface 953B partially covered by anchor portions 1367, it will be understood that in some examples, the entire (or substantially the entire) non-stimulation surface 953B may be covered by anchor portions 1367.

As further shown in FIG. 15C, in some examples the anchor portions 1367 may comprise a thickness T3 (e.g., height) which is less than a distance T4 (e.g., height) by which electrodes 956 may protrude from first surface 953A such that the anchor portions 1367 may enhance securing the stimulation element but have a low profile to also help facilitate robust engagement of the electrodes 956 with the target tissue. In some examples, the anchor portions 1367 in the example of FIG. 15C may have a thickness T5 (e.g., height) which is substantially thicker than (e.g., greater than) the low profile thickness T3 (e.g., height) of the anchor portions 1367 so that anchor portions 1367 (on the non-stimulation surface 953B) may provide for more aggressive engagement of surrounding tissue.

FIG. 16A is a diagram 1450 including a top plan view schematically representing an example paddle-like body 954 comprising an array of anchor portions 1417 distributed in a pattern spaced apart from each other on a first surface 953A (e.g., stimulation surface) of the body 954, with at least some of the various anchor portions 1417 interposed between adjacent electrodes 956 such that the anchor portions 1417 are spaced apart from each other in a first orientation parallel to a length (e.g., a longitudinal axis LA) of the body 954. The anchor portions 1417 also are spaced apart from each other in a second orientation (SO) perpendicular to the first orientation, with such rows 1419 of anchor portions extending generally parallel to a length of the electrodes 956.

FIG. 16B is a diagram 1475 including a top plan view schematically representing an example device (and/or example method) including paddle-like body 954 comprising an array 1476 of anchor portions 1477 distributed in a pattern of columns spaced apart from each other on an opposite second surface 953B (e.g., non-stimulation surface) of the body 954, with at least some of the various anchor portions 1477 spaced apart from each other in a second orientation (SO) perpendicular to a length (e.g., a longitudinal axis LA) of the body 954. Each anchor portion 1477 extends generally perpendicular to the length of the electrodes 956 and extends generally parallel to the length (L2) of the body 954. In some examples, the anchor portions 1477 may comprise at least some of substantially the same features and attributes as anchor portions 4017 of the example arrangement in FIG. 16A, except for comprising a different shape, size, and/or orientation.

FIG. 16C is a diagram 1785 including a top plan view schematically representing an example device (and/or example method) including a paddle-like body 954 comprising an array 1486 of anchor portions 1487 distributed in a pattern spaced apart from each other in a generally parallel relationship on an opposite second surface 953B (e.g., non-stimulation surface) of the body 954. In some examples, the anchor portions 1487 may sometimes be referred to as extending diagonally across the body 954. In some examples, in this diagonal configuration, the various anchor portions 1487 extend in long strips which may enhance securing the stimulation element in (or generally parallel to) both a major axis orientation (e.g., lengthwise orientation, along longitudinal axis LA) and a minor axis orientation (e.g., transverse orientation SO) of the body 954. In some examples, the anchor portions 1487 may comprise at least some of substantially the same features and attributes as anchor portions 1477 of the example arrangement in FIG. 16B, except for comprising a different shape, size, and/or orientation.

In any of the above (and below) described examples of a stimulation element having fixation arrangements, strain relief may be provided using a variety of techniques. In some examples, strain relief may be provided by looping the lead body or another portion of the stimulation element between electrodes and an anchoring point (or anchor portion) of the fixation arrangement. In some examples, strain relief may be provided by the flexibility, e.g., stretch, of the lead body and/or other portion of the stimulation element. As an example, the lead body may exhibit twenty percent or more elongation with 5 Newton (N) of strain force applied as compared to no strain. In some examples, a portion of the lead body or other portion of the stimulation element may have a non-straight geometry between the electrodes and the fixation arrangement, and in response to strain, the non-straight geometry can expand or straighten to effectively elongate the length of the lead body and/or other portion of the stimulation element. For example, the lead body may include a pre-formed strain relief segment 3119 as previously described in connection with FIG. 6C.

FIG. 16D is a diagram 4090 including a top plan view schematically representing an example paddle-like body 954 comprising at least some of substantially the same features and attributes as the paddle-like body 954 of FIG. 16A (and/or 16B, 16C), except further comprising an array 4093 of anchor portions 4094 located on a periphery or outer side edge 4092 of the body 954. In some examples, the anchor portions 4094 may comprise at least some of substantially the same features and attributes as anchor portions (e.g., 1417, 1477, etc.) of the example fixation arrangement in FIGS. 16A, 16B, etc., respectively, except for comprising a different shape, size, and/or orientation as represented by FIG. 16D.

In some examples, the respective anchor portions 4094 are spaced apart from each other about the periphery 4092 of paddle-like body 954, which may provide a desired combination of slidability for initial positioning and for fixation once the paddle-like body 954 has been maneuvered into a location of chronic implantation. However, in some examples, the respective anchor portions 4094 are provided with little or no spacing between respective anchor portions 4094 such that the periphery 4092 may be considered to comprise a continuous or substantially continuous anchor portion.

In one aspect, in some examples the example arrangement periphery-located anchor portions 4094 of FIG. 16D may enhance anchoring within or among certain types of tissues while potentially lessening an amount of the surface area of other portions (e.g., 953A, 953B) of a body 954 to be partially covered with some anchor portions. In another aspect, in some examples such arrangements may enhance anchoring for certain orientations (e.g., anterior-posterior, superior-inferior, medial-lateral) in view of a direction, orientation, etc. in which muscle portions of the target tissues (or surrounding non-target tissues) may move.

FIG. 16E is a diagram 4300 including a top plan view of an example flexible connector segment 4306 which may comprise at least some of substantially the same features and attributes as (or comprise an example implementation of) as further described herein, flexible connector segments or distal lead segments (FIGS. 18A-22B) extending between the respective bodies 954, while also comprising fixation arrangement 4320 extending along a length of the flexible connector segment 4306.

As shown in FIG. 16E, the fixation arrangement 4320 forms a helical pattern on an exterior surface 4312 of the flexible connector segment 4306, with the fixation arrangement 4320 comprising anchor portions 4322 and anchor portions 4323 (shown in dashed lines to represent an opposite side of the flexible connector segment 4306).

In some examples, the anchor portions 4322 and the anchor portions 4323 may be spaced apart from each other by some distance, while in some examples, the anchor portions 4322 and anchor portions 4323 form part of a single, continuous fixation arrangement.

In some examples, each anchor portion comprises a plurality of fixation elements, which comprise at least some of substantially the same features and attributes as the anchor portions, fixation elements, etc. as described in association with at least FIGS. 14A-14B, 15A-15C in which a plurality of fixation elements are configured to engage surrounding tissues (e.g., target tissues and/or non-target tissues) to secure the flexible connector segment (or distal lead segments) relative to surrounding tissues. This arrangement 4320 also acts to secure associated stimulation elements relative to the target tissues such as nerve portions, muscle portions, combinations of nerve portions and muscle portions, neuromuscular junctions of nerve portions and muscle portions, and/or combinations thereof.

In considering the various anchor portions described throughout the examples of at least FIGS. 14A-22B, it will be understood that anchor portions may be located on just the stimulation electrode arrangement, on just the flexible connector segments (or distal lead segments), or on both the stimulation elements and the flexible connector segments (or distal lead segments).

While FIGS. 15A-16E show fixation elements on stimulation element comprises paddle-like bodies, similar type fixation elements may be formed on other types of stimulation elements, such as on the lead of an axial electrode array.

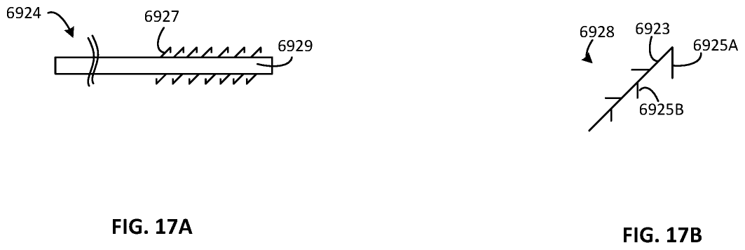

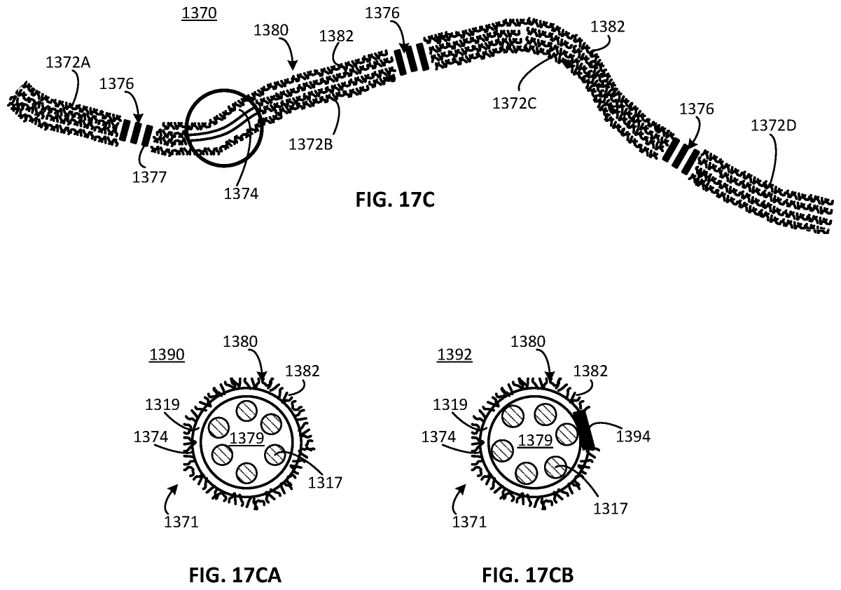

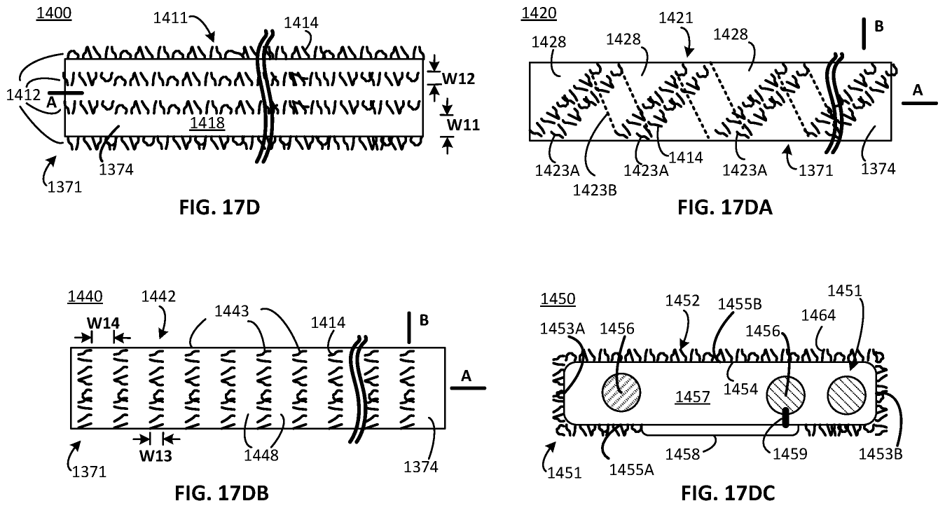

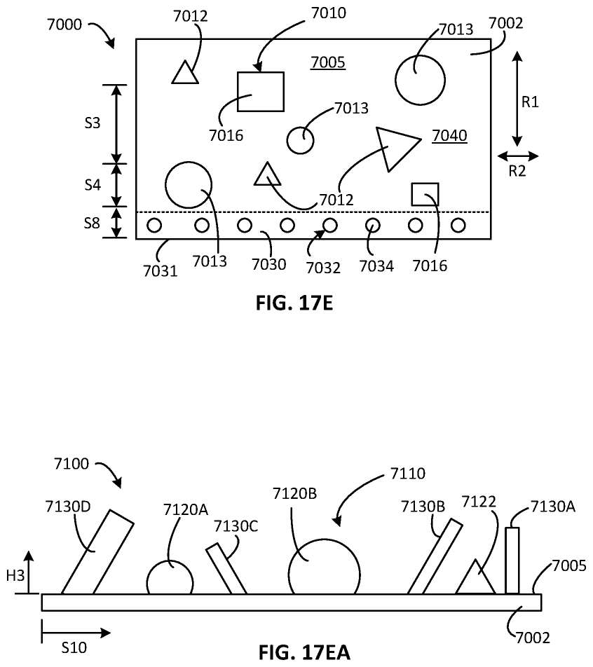

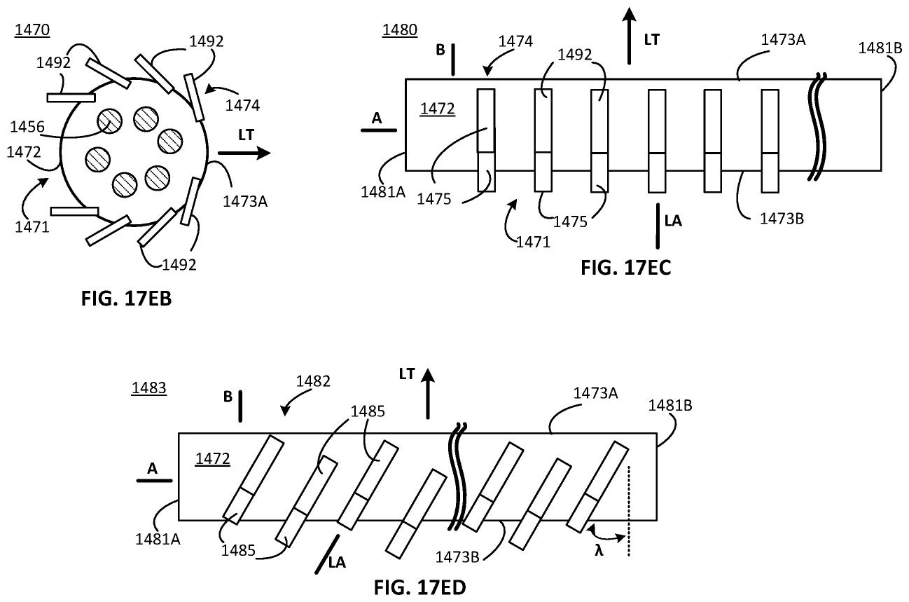

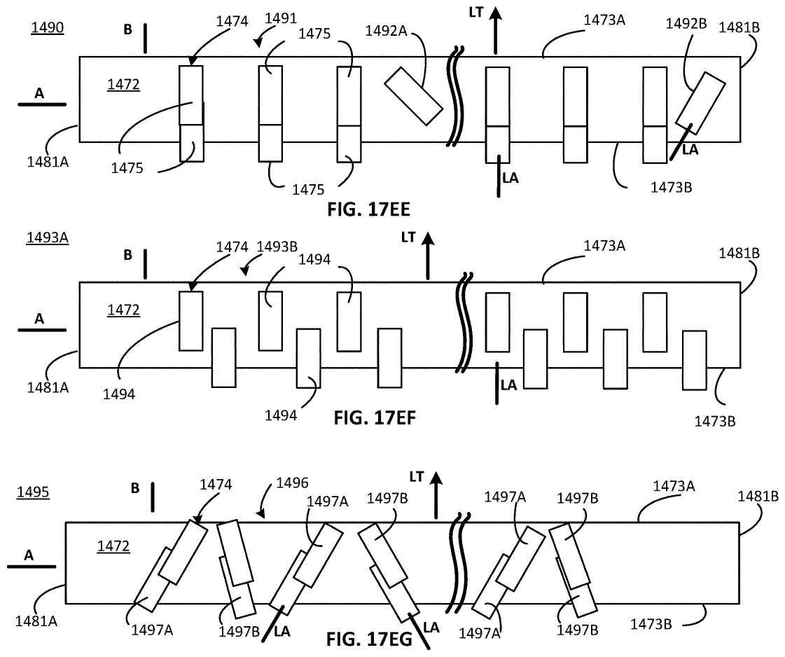

With this in mind, in some examples, the fixation elements may be on other portions of stimulation elements, such as on the lead body of a lead. For example, FIGS. 17A-17EG show different example fixation elements, which may be on various types of stimulation elements, with FIGS. 17C-17DC showing examples of fixation elements on at least portions of a lead body of a lead of a stimulation element.

FIG. 17A-17B illustrate example fixation elements. In particular, FIG. 17A is a greatly enlarged side view of just one fixation element 6924 and, in some examples, at least some (or all) of the fixation elements (as illustrated by 6924) of a fixation arrangement may comprise protrusions 6927 on their surfaces, which in some examples may comprise barbs, hooks, or other sharp tipped structures. In some examples, the protrusions 6927 may be present on just a portion of the fixation element 6924, such as but not limited to a distal portion 6929 of the fixation element 6924. However, in some examples, the protrusions 6927 may be present on the entire or substantially entire surface of the fixation element 6924. In yet other examples, groups of protrusions 6927 may be positioned in spaced apart clusters, which are spaced apart from each other along and around the surface of the fixation element 6924.

It will be further understood that the protrusions 6927 are not strictly limited to structures having a sharp-tip or hook but may comprise structures comprising a rounded edge while including a sticky surface coating or formed as a non-sharp tipped member which can securely engage a surrounding non-nerve tissue in close proximity to a target stimulation site.

FIG. 17B is a diagram including a side view schematically representing an example protrusion 6928. In some examples, the protrusion 6928 may comprise at least some of substantially the same features and attributes as protrusion 6927 described in association with at least FIG. 17A and/or may comprise an example implementation of protrusion 6927. As shown in FIG. 17B, in some examples protrusion 6928 may comprise a main fixation element 6923 for protruding outward (e.g., biased to extend outwardly at an angle) from an outer surface of a lead to function as part of a fixation arrangement, with protrusion 6928 including a first secondary fixation element 6925A extending at an angle relative to the main fixation element 6923. The combination of the first secondary fixation element 6925A and the main element 6923 may sometimes be referred to as a barb at least to the extent that the respective main and secondary fixation elements 6923, 6925A form a sharp point with the secondary fixation element 6925A having an orientation which is at least partly opposite of the general orientation of the main fixation element 6923. In some examples, the protrusion 6928 may further comprise additional secondary fixation elements 6925B spaced apart from each other along a length of the main fixation element 6923 and also extending outward at angle relative to the main fixation element 6923. In some examples, each secondary fixation element 6925B also may comprise a barb, e.g., a further protrusion extending at an angle relative to the secondary element.

FIGS. 17C-17CB illustrate example fixation arrangements comprising a lead or other body 1370, herein generally referred to as a “stimulation portion 1370”. In some examples, the stimulation portion 1370 may comprise at least some of substantially the same features and attributes as, and/or an example implementation of, the example stimulation elements described in association with at least FIGS. 1-17B and/or an example implementation of such previously described stimulation elements.

As further shown in FIG. 17C, in some examples, stimulation portion 1370 comprises a fixation arrangement 1380 which extends along and around the entire or substantially the entire outer surface 1374 of the stimulation portion 1370 with at least some stimulation electrode arrangement(s) 1376 interposed between segments of the fixation arrangement 1380 that include fixation elements 1382. In some examples, the stimulation portion 1370 may include a lead body, a flexible connector segment, or other elongated portion of the stimulation element 1370. Each stimulation electrode arrangement 1376 may comprise at least one stimulation electrode 1377. Among other aspects, the fixation arrangement 1380 stands in contrast to some leads which merely include a limited number of discrete fixation elements. Instead, the fixation arrangement 1380 provides a continuous or substantially continuous coverage of fixation elements 1382 on outer surface 1374 of the stimulation portion 1370. In some examples, the substantially continuous coverage may comprise covering at least about 50 percent of the total surface area of the outer surface 1374 of the stimulation portion 1370. In some examples, the substantially continuous coverage may comprise at least about 60 percent, at least about 65 percent, at least about 70 percent, at least about 75 percent, at least about 80 percent, at least about 85 percent, at least about 90 percent. In some examples, the stimulation electrode arrangements 1376 or portions thereof may include sensing electrodes for sensing information, as described herein.

In some examples, the continuous or substantially coverage of outer surface 1374 with fixation elements 1382 may sometimes be referred to as a region of indefinite number of fixation elements 1382.

Among other aspects, the fixation arrangement 1380 may facilitate robust fixation of the lead segments 1372A, 1372B, 1372C, 1372D, etc. and/or stimulation electrode arrangements 1376 relative to surrounding tissues. At the same time, the relatively low profile of the fixation arrangement 1380 permits at least lateral advancement and maneuvering of the lead segments and/or the stimulation electrode arrangements 1376 into the implant positions (and orientations), such as the deployments further illustrated in connection with FIGS. 18A-22B.

FIG. 17CA is a diagram 1390 including a sectional view schematically representing one example implementation of the stimulation portion 1370 of FIG. 17C. As shown in FIG. 17CA, the example stimulation portion 1370 may comprise at least some of substantially the same features and attributes as previously described in association with FIG. 2F and/or leads as described by FIGS. 4-6C.

As further shown in FIG. 17CA, the example stimulation portion 1370 comprises a fixation arrangement 1380, which includes a plurality of fixation elements 1382 which are formed on, or defined as part of, the outer surface 1374 of an outer wall 1319 one of the lead segments (e.g., 1372A, 1372B, etc.), which define at least part of the stimulation portion 1370 (FIG. 17C). In some examples, the fixation elements 1382 define a generally uniform pattern covering the entire or substantially the entire outer surface 1374 of the lead segment(s) (e.g., 1372A, 1372B, etc.) of the portion of the stimulation portion 1370.

FIG. 17CB is a diagram 1392 including a sectional view schematically representing an example implementation of the portion of the stimulation portion 1370 of FIG. 17C (and sectional view of FIG. 17CA), while including a stimulation electrode 1394 in electrical connection with one of the electrical conductors 1317 extending within an interior 1379 of one of the lead segments (e.g., 1372A, 1372B, etc.) of stimulation portion 1370. As shown in FIG. 17CB, in some examples, the fixation elements 1382 may at least partially surround the stimulation electrode 1394.

In some examples, the fixation elements of the fixation arrangements may form or be a pad, a layer and/or a sheet, such as illustrated by the fixation elements 1382 of FIGS. 17C-17CB. The pad, layer, and/or sheet may be in different patterns and/or not cover the entire outer surfaces 1374 in some examples, as further illustrated herein.

Each of FIGS. 17D-17DC is a diagram including a side view schematically representing an example stimulation portion (or portion of a stimulation lead body) including a fixation arrangement formed on, or defined at least partially by, an outer surface of the stimulation portion (or of the stimulation lead body). In some examples, each example fixation arrangement (1411 in FIG. 17D; 1421 in FIG. 17DA; 1442 in FIG. 17 DB; 1452 in FIG. 17DC) may comprise at least some of substantially the same features and attributes of a fixation arrangement (and its associated stimulation portion or portion of a lead body) of the examples described in association with at least FIGS. 17A-17CB, or may comprise an example implementation of the fixation arrangement (and its associated stimulation portion or portions of a stimulation lead body) described in association with at least FIGS. 14A-14B, 15A-15C, and 16A-16E. It will be further understood that such example fixation arrangements also may be incorporated into other example devices of the present disclosure, such as on an outer surface of at least a portion of a stimulation lead body, stimulation portion, other type of fixation element, etc.

As shown in the diagram 1400 of FIG. 17D, in some examples fixation arrangement 1411 may comprise a plurality of rows 1412 of fixation elements 1414 formed on (or defined as at least part of) an outer surface 1374 of a stimulation portion (or portion of a lead body) with spacing 1418 (e.g., absence of fixation elements 1414) interposed between adjacent rows 1412 of the fixation arrangement 1411. In this arrangement, the rows 1412 are circumferentially spaced apart. In one aspect, each row 1412 is aligned with (e.g., generally parallel to) a longitudinal axis (represented by line A) of the stimulation portion 1371 (or lead body). In some such examples, the size (e.g., width W11) of spacing 1418 and size (e.g., width W12) of the rows 1412 may be selected to implement a desired percentage of coverage of the surface area on the outer surface 1374 of the stimulation portion 1371. However, even with the spacing 1418, in some examples the fixation arrangement 1411 may sometimes be referred to as extending or covering the entire (or substantially the entire) length of the stimulation portion 1371 (or portion of lead body) and/or may form pads. It will be further understood that even with the inclusion of some minor interruptions (e.g., spaces) along a length of a row 1412 of the fixation arrangement 1411, the row 1412 (and fixation arrangement) may still be considered to extend the entire length (or substantially entire length) of the stimulation portion (or portion of stimulation lead body). For instance, one such non-limiting example of an interruption may comprise the presence of a stimulation electrode arrangement (e.g., an array of stimulation electrodes) which is located along the length of the rows(s) 1412 of the fixation arrangement 1411.

With regard to the examples of at least FIGS. 17D-17DC, in some examples a plurality of fixation elements provide substantially continuous coverage (e.g., occupy a surface area) on an outer surface of at least one of a lead body or other portion of a stimulation element. In some such examples, the substantially continuous coverage comprises at least about 25 percent coverage, at least about 30 percent coverage, at least about 35 percent coverage, at least about 40 percent coverage, at least about 45 percent coverage, at least about 50 percent coverage, at least about 60 percent coverage, at least about 65 percent coverage, at least about 70 percent coverage, at least about 75 percent coverage, at least about 80 percent coverage, at least about 85 percent coverage, at least about 90 percent coverage, or at least about 95 percent coverage of the outer surface of at least one of a lead body, a stimulation portion (including distal lead segments and/or a stimulation element), or other portion of a stimulation element. It will be further understood that these examples of substantially continuous coverage may be applied to examples of the present disclosure regarding a plurality of fixation elements other than FIGS. 17D-17DC.

With regard the example of at least FIG. 17D in which rows 1412 extend longitudinally along length of a lead body, stimulation portion, and/or a stimulation element, the rows 1412 are spaced apart from each other circumferentially, wherein spacing between adjacent rows 1412 comprises an arc length about 5 to about 10 degrees, of about 10 to about 20 degrees, of about 20 to about 30 degrees, of about 30 to about 40 degrees, of about 40 to 50 degrees, of about 50 to about 60 degrees, of about 60 to 70 degrees, of about 70 to about 80 degrees, of about 80 to about 90 degrees, or of about 90 to about 120 degrees.

As shown in the diagram 1420 of FIG. 17DA, example fixation arrangement 1421 may comprise at least some of substantially the same features and attributes of the fixation arrangement 1411 of FIG. 17D, except with the fixation elements 1414 arranged in a helical pattern of strips 1423A extending about the outer surface 1374 with spacing 1428 (e.g., absence of fixation elements 1414) interposed between adjacent strips 1423A. The dashed lines 1423B represent anchor strips on a backside of the stimulation portion not visible in the view of FIG. 17DA, with strips 1423B being in general continuity with strips 1423A, in some examples. Among other aspects, the helically-patterned fixation arrangement 1421 may provide a desirable combination of sufficient anchorability in both the lateral and longitudinal orientations, while also permitting enough slidability in both the lateral and longitudinal orientations to facilitate implementing desired positioning of the stimulation electrodes of a stimulation portion at implant locations of target tissues. The helically-patterned fixation arrangement 1421 may sometimes be referred to as a spiral pattern. In some examples, spacing between adjacent turns about the outer surface 1374 may comprise at least some of substantially the same features regarding coverage and/or spacing as described in association with at least FIGS. 17D and 17DB.

As shown in the diagram 1440 of FIG. 17 DB, example fixation arrangement 1442 may comprise at least some of substantially the same features and attributes of the fixation arrangement 1411 of FIG. 17D, except with the fixation elements 1414 on outer surface 1374 arranged in rows 1443 aligned perpendicular to the longitudinal axis (A) of the stimulation portion (or portion of lead body) with spacing 1448 (e.g., absence of fixation elements 1414) interposed between adjacent rows 1443 of fixation elements 1414. In some examples, the particular fixation arrangement 1421 may enhance longitudinal slidability while resisting lateral slidability, particularly after implantation.

In some such examples associated with FIG. 17DB, the rows 1443 extend circumferentially with each row 1443 extending transverse to a longitudinal axis of lead (and/or stimulation element), at least in the region in which the rows 1443 are located, with the rows 1443 being spaced apart from each other longitudinally. In some such examples, the spacing (W14) between adjacent rows 1443 comprises at least one multiple, at least two multiples, or at least three multiples of a width (W13) of each row 1443.

FIG. 17DC is a diagram 1450 including a sectional view schematically representing an example fixation arrangement 1452 for a stimulation element 1451A. As shown in FIG. 17DC, example fixation arrangement 1452 may comprise at least some of substantially the same features and attributes of (and/or an example implementation of) the fixation arrangements as described in association with at least FIGS. 14A-14B, 15A-15C, 16A-16E, 17A-17CB, with fixation arrangement 1452 deployed on an outer surface 1454 of a housing of the stimulation electrode arrangement 1451 having at least one stimulation electrode 1458. As shown in FIG. 17DC, in some examples the fixation arrangement 1452 comprises a plurality of fixation elements 1464 (like fixation elements 1414) extending over the surface area of the entire (or substantially the entire) outer surface 1454 of the stimulation electrode arrangement 1451, including upper and lower surfaces 1455A, 1455B, and side surfaces 1453A, 1453B, (and end surfaces not seen in the sectional view). As further seen in FIG. 17DC, electrical conductors 1456 extend within and through the interior 1457 of the stimulation electrode arrangement 1451 with a respective one of the conductors 1456 being electrically connected (via link 1459) to the stimulation electrode 1458 on lower surface 1455A of the stimulation electrode arrangement 1451. Like the fixation arrangement present on lead segments (which extend between adjacent stimulation electrode arrangements), the fixation arrangement 1452 on an outer surface 1454 of the stimulation element as in FIG. 17DC may enhance securely fixing the stimulation electrode arrangement 1451 in a position of stimulating relation to target tissues.

In some examples, the fixation arrangements described in association with at least FIGS. 14A-14B, 15A-15C, 16A-16E and 17A-17DC may be implemented according to at least some of substantially the same features and attributes as fixation arrangements 7000, 7100 described in association with FIGS. 17E-17EG.

FIG. 17E is a diagram including an enlarged top view schematically representing an example fixation arrangement 7000 formed on, and including as part of the fixation arrangement 7000, a base 7002. In some examples, the fixation arrangement 7000 may provide a matrix of heterogeneous fixation elements. However, in some examples, the fixation arrangement 7000 of FIG. 17E may have wide applicability to act as an anchor or position-influencing element. In some examples, the fixation arrangement 7000 in FIG. 17E may comprise an example implementation of the fixation arrangements, portions, fixation elements in the examples in association with at least FIGS. 14A-14B, 15A-15C, 16A-16E, and 17A-17DC and may comprise at least substantially the same features and attributes as the fixation arrangements, portions, fixation elements, etc. in the examples in association with at least FIGS. 14A-14B, 15A-15C, 16A-16E, and 17A-17DC.

As shown in FIG. 17E, the fixation arrangement 7000 may comprise an array 7010 of example heterogeneous fixation elements 7012, 7013, 7016 formed on (and/or extending upward from) a surface 7005 of base 7002. In some examples, the surface 7005 may comprise a planar surface and in some examples, the surface 7005 may comprise a non-planar surface. Together, the heterogeneous fixation elements 7012, 7013, 7016 may form a matrix, network, or the like which may overlap or otherwise be juxtaposed relative to each other to create a generally traction-favoring surface profile. It will be understood that in some examples, the various heterogeneous fixation elements of array 7010 may be positioned much closer to each other than shown in FIG. 17E in order to touch, overlap, partially interlock or interfere with each other, etc. so as to increase the frictional properties (e.g., slide-resistance) of the fixation arrangement 7000 or to reduce the frictional properties (e.g., slidability) of the fixation arrangement 7000, depending on the type, size, orientation, coating, etc. of the particular arrangement of fixation elements of the array 7010.

In general terms, the various fixation elements of the array 7010 may comprise a flexible, resilient material. However, depending on the goals regarding slidability or slide-resistance, some elements may be firmer or softer.

In some examples, the particular types, spacing between, orientation, position, relative flexibility, etc., of the heterogeneous fixation elements of the array 7010 may be selected and formed to correspond to a selectable coefficient of kinetic friction to enable a desired bias for controlled slidable movement relative to tissues within a patient's body and/or relative to lumen within a patient's body and/or to correspond to a selectable coefficient of static friction to enable a desired bias to remain statically positioned at a chose location relative to tissues or within a lumen.

In some examples, whether or not expressed formally as a coefficient of kinetic or static friction, the various heterogeneous fixation elements of the array 7010 are selected and formed according to their height, size, shape, position, spacing, orientation relative to each other, relative flexibility, etc. to create a desired anchoring effect while still permitting some degree of slidable advancement.

As shown in FIG. 17E, at least some example shapes (as seen in cross-section from a top view) may comprise fixation elements with shapes which are triangular 7012, circular 7013, rectangular 7016, and the like. The fixation elements also may have different sizes (e.g., diameter, greatest cross-sectional dimension, width, and the like such as represented by S4), and spacing (e.g., S3) between each other or relative to an edge 7031 (e.g., S8) of the base 7002. In some examples, at least some of the fixation elements of array 7010 may comprise hook-shapes, J-shapes, U-shapes, etc. In some examples, at least some of the fixation elements or the juxtaposed pattern of such fixation elements, may promote tissue in-growth and long term fixation, such as but not limited to, apertures formed in such fixation elements or by the juxtaposition of some of the respective elements.

The various fixation elements also may be organized in directional patterns, such as being in rows aligned in a first orientation (R1) or second orientation (R2) which are orthogonal to each other, or in other non-orthogonal orientations. Such orientations may be used to effect selectable bias to permit or prevent slidable movement in various directions, which may enhance positioning and/or anchoring of the medical element on which the fixation arrangement 7000 is located.

In some examples, at least some fixation elements of the array 7010 may be arranged along a periphery 7030 of the base 7002 in a row or other organizational pattern. The fixation elements 7034 in one example row 7032 may have the same height, size, shape, positions, etc., or may have heights, sizes, shapes, positions different from each other. By providing this configuration along one or more edges 7031 of the base 7002, the fixation arrangement 7000 may influence slidability or slide-resistance in particular directions. In a related aspect, the presence or absence of fixation elements of array 7010 in an interior portion 7040 also may provide analogous influences, with or without the edge-type rows, etc. of such elements.

In some examples, the interior portion 7040 of the base 7002 and/or the elements of array 7010 also may comprise a coating with desired lubricous and/or frictional qualities, which may be selected to work synergistically with the various shapes, sizes, positions, spacing, orientation, etc. of the elements of array 7010.

FIG. 17EA is a diagram including an enlarged side view schematically representing an example fixation arrangement 7100 formed on, and including as part of the fixation arrangement, a base 7002. In some examples, the fixation arrangement 7100 may provide a matrix or network of heterogeneous fixation elements. However, in some examples, the fixation arrangement 7100 may have wide applicability to act as an anchor or position-influencing element.

In some examples, the fixation arrangement 7100 in FIG. 17EA may comprise at least some of substantially the same features and attributes as fixation arrangement 7000 in FIG. 17E.

As shown in FIG. 17EA, the fixation arrangement 7100 comprises an array 7110 of fixation elements comprising different shapes, sizes (e.g., heights, diameters, etc.), positions, spacing, orientations, etc. For example, rectangular fixation elements 7130A, 7130B, 7130C, 7130D exhibit differing angular orientations (e.g., relative to a horizontal plane through which base 7002 extends), which may sometimes be referred to as being bi-directional or multi-directional. Other fixation elements may comprise spherical shaped elements 7120A, 7120B, pyramid-shaped elements 7122, etc. The respective fixation elements of array 7110 may be formed according to a selectable height (per height arrow H3), which may vary from each other as part of a desired effect to promote slidability or slide-resistance, depending on the intended use of the fixation arrangement and medical element to which is formed/attached.

It will be further understood that some shapes, such as the spherical fixation elements 7120A, 7120B may be more likely to enhance slidability because of their smooth convex surface while some shapes, such as the pyramid fixation element 7122 or rectangular fixation elements (7130A-7130D), may enhance slide-resistance, depending on their orientation. In some examples, directional arrow S10 may represent relative horizontal spacing between elements of array 7110.

In some examples, the base 7002 may formed in a two-dimensional plate shape such that the fixation arrangement 7000 (FIG. 17E) or 7100 (FIG. 17EA) may be readily formed or attached to a back side of a carrier opposite to an electrode side of a stimulation portion, such as a paddle-shaped carrier which carries contact electrodes. However, in some examples, the base may comprise a cylindrical shape such that the fixation elements of array 7010 (FIG. 17E) and/or array 7110 (FIG. 17EA) may extend circumferentially outward from a cylindrically shaped lead on which the array 7010 (FIG. 17E) or 7110 (FIG. 17EA) is formed or attached. Examples are not so limited and the base may comprise other shapes, as well.

In some examples, and with general reference to anchoring examples in association with at least FIGS. 14A-14B, 15A-15C, 16A-16E, and 17A-17EA, a fixation arrangement comprising a plurality of fixation elements may comprise homogeneous fixation elements and/or heterogeneous fixation elements. In some such examples, at least a majority of the homogeneous fixation elements may comprise substantially the same size, shape, position, and/or orientation relative to each other. In some examples, the percentage of fixation elements which are homogeneous relative to each other may comprise at least about 55%, at least about 60%, at least about 65%, at least about 70%, at least about 75%, at least about 80%, at least about 85%, at least about 90%, or at least about 95%.

In some such examples, at least a majority of the heterogeneous fixation elements (e.g., FIGS. 17E-17EA) may comprise a different size, different shape, different position, and/or different orientation relative to each other. In some examples, the percentage of fixation elements which are heterogeneous relative to each may comprise at least about 55%, at least about 60%, at least about 65%, at least about 70%, at least about 75%, at least about 80%, at least about 85%, at least about 90%, or at least about 95%.

With regard to at least some of the example homogeneous fixation elements and/or example heterogeneous fixation elements of the present disclosure, each respective fixation element is separate from other respective fixation elements, and a quantity of the plurality of fixation elements is substantially different from, being greater than, at least one of: (A) a quantity of electrodes on at least one of: (1) a single stimulation electrode arrangement of multiple stimulation electrodes; and (2) all of the stimulation electrodes for a lead; and (B) a quantity of all the stimulation electrodes.

With regard to at least some of the example homogeneous fixation elements and/or example heterogeneous fixation elements of the present disclosure, at least some of the respective fixation elements extend outwardly from an external surface of a lead segment (or lead body) by a first distance which is substantially different from, being less than, at least one of a diameter of, a greatest cross-sectional dimension of, or a thickness of the lead segment (or lead body).

With regard to at least some of the example homogeneous fixation elements and/or example heterogeneous fixation elements of the present disclosure, at least some of the respective fixation elements extend outwardly from an external surface of a carrier body of the a stimulation element (e.g., a carrier body supporting contact electrodes) by a first distance which is substantially different from, being less than, at least one of a diameter of, a greatest cross-sectional dimension of, or a thickness of the stimulation element (e.g., the carrier body of the stimulation element).

With regard to at least some of the example homogeneous fixation elements and/or example heterogeneous fixation elements of the present disclosure, at least some of the respective fixation elements comprise a diameter or a greatest cross-sectional dimension which is substantially different from, being less than, a surface area of a respective one of the electrodes of the stimulation element. In some such examples, in this context the diameter (or greatest cross-sectional dimension) of the fixation elements is substantially less than a total surface area of all electrodes of a respective one of the first and second stimulation electrode arrangements and/or stimulation electrodes.

With regard to at least some of the example homogeneous fixation elements and/or example heterogeneous fixation elements of the present disclosure, in some examples the plurality of fixation elements may be located on a distal lead segment distal to a bifurcation portion of the lead body. In some such examples, the plurality of fixation elements may be located on a distal lead segment solely distal to a bifurcation portion of the lead body.

FIG. 17EB is a diagram 1470 including a sectional view schematically representing an example stimulation portion 1471 (or portion of a stimulation lead body) of an example device and/or example method comprising at least some of substantially the same features and attributes as (but not limited to) the examples described in association with at least FIGS. 15A-17EA, except comprising a fixation arrangement 1474 comprising a plurality of tines 1492 extending about the outer surface 1472 of the stimulation portion 1471. As shown in FIG. 17EB-17EC, the tines 1492 extend generally perpendicular to a longitudinal axis (reference line A) of the stimulation portion 1471 (or portion of stimulation lead body) and parallel to a minor axis (reference line B) of the stimulation portion 1471.

Moreover, as further shown in FIG. 17EB-17EC, in some examples, the stimulation portion (or portion of stimulation lead body) 1471 may be advanced within the patient's body in an orientation (represented by directional arrow LT) which is lateral (e.g., transverse) to a longitudinal axis (line A) of the stimulation portion 1471, which stands in contrast to the typical advancement of a stimulation lead portion in alignment with a longitudinal axis (A) of the stimulation portion 1471. Accordingly, the tines 1492 are aligned to enhance lateral stability of the stimulation portion 1471 more substantially than longitudinal stability of the stimulation portion 1471 while also making the stimulation portion 1471 more maneuverable in a lateral orientation (as represented by directional arrow LT) in order to advance and place the stimulation lead portion(s), in which the stimulation electrode arrangements and lead segments of the stimulation portion are implanted such as with minor tunneling or no longitudinal tunneling, such as via a direct visualization of the target tissues at which the stimulation electrode arrangements (and supporting lead segments) may be maneuvered more directly to their implant locations at which stimulating relation (relative to target tissues) is established.

As in other examples, electrical conductors 1456 may extend through and within an interior of the stimulation portion (or portion of stimulation lead body) 1471.

In some examples, the fixation arrangement may sometimes be referred to as generally providing sideways tines (e.g., being oriented laterally) in at least some lead segments of a stimulation portion versus longitudinal-oriented tines.

FIG. 17EC is a diagram 1480 including a top plan view schematically representing the stimulation portion 1471 of FIG. 17EB. As shown in FIG. 17EC, tines 1492 are spaced apart from each other along a length (e.g., longitudinal axis A) of the stimulation portion 1471 with a longitudinal axis of tines 1492 aligned with an expected generally lateral orientation (LT) (versus a more traditional longitudinal orientation) of advancement of the stimulation portion 1471 within the patient's body during at least some of the implantation of the stimulation portion 1471 in some examples. FIG. 17ED also further illustrates the stimulation portion 1471 having opposite ends 1481A, 1481B and opposite sides 1473A, 1473B.

FIG. 17ED is a diagram 1483 including a top plan view like that of FIG. 17EC schematically representing an example stimulation portion 1482 comprising at least some of substantially the same features and attributes as the stimulation portion 1471 of FIG. 17EB-17EC, except with tines 1485 (like tines 1492) arranged at a slant (e.g., an angle λ) such that a length (e.g., longitudinal axis LA) of the tines 1485 are not perpendicular to the longitudinal axis (line A) of the stimulation portion 1471 or not parallel to the minor axis B of the stimulation portion 1471. The angle λ is selected such that the tines 1485 help to resist “backing out” of the stimulation portion 1491 from an implanted location along the lateral orientation LT (along or parallel to line B) while simultaneously preventing any significant shifting of the stimulation portion 1491 in the longitudinal orientation (along line A).

Via this arrangement, the angled tines 1485 may facilitate slidable advancement of the stimulation portion 1482 in the lateral orientation LT by which a length of the stimulation portion 1482 (or portion of a stimulation lead body) may be inserted and advanced within a patient's body to become positioned at an implant location in stimulating relation to a target tissue (e.g., target nerve portion, target muscle portion, and/or neuromuscular junction) to increase or maintain upper airway patency. In addition, the angled tines 1485 help to maintain longitudinal stability of the stimulation portion at the implant location relative to target tissue.

FIG. 17EE is a diagram 1490 including a top plan view like that of FIG. 17EC-17ED schematically representing an example stimulation portion 1491 comprising at least some of substantially the same features and attributes as the stimulation portion 1471 of FIG. 17EC-17ED, except with the addition of at least some tines 1492A, 1492B (like tines 1492) arranged in at angle like tines 1485 in FIG. 17EE with some tines (e.g., 1492A) oriented divergently from some tines (e.g., 1492B). Among other aspects, this example arrangement of providing some of the tines 1492A, 1492B at angle (like angle λ in FIG. 17ED) but in different orientations may provide a more robust fixation in some implementations by providing some back-out resistance in divergent orientations.

FIG. 17EF is a diagram 1493A including a top plan view like that of FIG. 17EC-17ED schematically representing an example stimulation portion 1493B comprising at least some of substantially the same features and attributes as the stimulation portion 1471 of FIG. 17EC-17ED, except with tines 1494 (like tines 1492) arranged in a staggered relationship in a lateral insertion orientation (LT) such that a length of some of the tines 1494 are offset from each other in the circumferential orientation. Among other aspects, this example arrangement of tines may provide a more robust fixation in some implementations by providing more variability in anchor points in both a circumferential orientation (along or parallel to line B) and longitudinal orientation (along or parallel to line A). In some such examples, the example fixation arrangement may inhibit or prevent longitudinal migration of the stimulation portion 1493B, which sometimes may be referred to as “lead ratcheting” or “inch worming.” Similarly, the example fixation arrangement may inhibit or prevent lateral migration of the stimulation portion 1493B.

FIG. 17EG is a diagram 1495 including a top plan view like that of FIG. 17EC-17ED schematically representing an example stimulation portion 1496 comprising at least some of substantially the same features and attributes as the stimulation portions of 17EC-17ED, except with tines 1497A, 1497B (like tines 1492A, 1492B in FIG. 17EG) arranged in at angle (like angle λ in FIG. 17EF) but in a divergent orientation relative to each other. Among other aspects, this example arrangement of tines may provide a more robust fixation in some implementations by providing by providing some back-out resistance in divergent orientations.

In some examples, a fixation arrangement for a stimulation portion (e.g., lead body, stimulation element) of various examples of the present disclosure and/or of a pulse generator may comprise varying combinations of the features and attributes of the example implementations of FIG. 17EB-17EG.

Moreover, in some examples, a stimulation portion may be implemented to comprise a fixation arrangement comprising at least some of substantially the same features of the fixation arrangement of FIG. 17EB-17EG combined with at least some of substantially the same features and attributes of the fixation arrangement(s) of at least FIGS. 14A-17EA.

FIGS. 18A-22B are diagrams schematically representing deployment of example stimulation elements or portions thereof of FIGS. 7A-17EG. In some examples, the stimulation elements illustrated in connection with FIGS. 18A-22B may be an example implementation of the stimulation elements of FIGS. 1F-1G or FIGS. 4-6C, and/or may be used to implement the method 10 illustrated by FIG. 1A and/or the method illustrated by FIGS. 2A-2G. The various stimulation elements may be implanted in stimulating relation to IHM-related tissue in a head-and-neck region of the patient to stimulate the IHM-related tissue and affect upper airway patency.

FIGS. 18A-18C illustrate example deployments 1501, 1505, 1513 of a stimulation element 1507 comprising a pair of heads 1510A, 1510B carrying stimulation electrodes 1506. In some examples, each of the heads 1510A, 1510B may include an implementation of and/or include at least some of substantially the same features and attributes as the heads 614, 644 of stimulation elements 630, 631 of FIGS. 7A-7B. The common features and attributes are not repeated for clarity.

As shown in FIGS. 18A-18C, the stimulation element 1507 may be implanted at or near to IHMs 244, 254 in a head-and-neck region of the patient. In some examples, the pair of heads 1510A, 1510B of the stimulation element 1507 may formed in or include a U-shape, which may allow for the heads 1510A, 1510B to be placed around at least a portion of least one IHM 244, 254.

As shown by FIG. 18A, in some examples, the heads 1510A, 1510B of the stimulation element 1507 may be positioned at or near and/or at least partially around the STM 244 on one or both sides of the patient such that the stimulation electrodes 1506A, 1506B, 1506C, 1506D arranged on the heads 1510A, 1510B of the stimulation element 1507 are in stimulating relation to the STM(s) 244 and/or to the IHM-innervating nerve. As shown, each head 1510A, 1510B includes two arms 1502A, 1502B extending from a base 1503, with the arms 1502A, 1502B including the stimulation electrodes 1506A, 1506B, 1506C, 1506D. For example, at least a portion of the stimulation electrodes 1506A, 1506B, 1506C, 1506D may be operated to apply a stimulation vector across (e.g., via electrode pairs) and through tissue, such as through the STM 244 and to capture the STM 244 and/or the IHM-innervating nerve. In some examples, each arm 1502A, 1502B of each head 1510A, 1510B may be independently addressable, such that the STM 244 on the left side and the STM 244 on the right side of the patient, and/or IHM-innervating nerve, may be selectively stimulated. In various examples, each of the arms 1502A, 1502B may include additional electrodes, which may be on the inside and/or the outside of the arms, and which may include stimulating electrodes and/or sensing electrodes. As shown by FIG. 18A, arms 1502A may be placed posterior to (e.g., behind) the STMs 244 and arms 1502B may be placed anterior to (e.g., in front of) the STMs 244. In some examples, the electrodes 1506A, 1506B, 1506C, 1506D or additional electrodes may be used to perform sensing, such impedance sensing between the electrodes for obtaining respiratory information as further described herein.

It will be understood that, via this example arrangement of FIG. 18A, the STM 244 is the sole IHM targeted by head 1510A for electrical stimulation (or for sensing), with STM 244 becoming sandwiched between the arms 1502A, 1502B of head 1510A such that the head 1510A. This arrangement stands in contrast to the following example of FIG. 18B, in which both the SHM 254 and the STM 244 are targeted by head 1510A for electrical stimulation (or for sensing), with both STM 244 and SHM 254 becoming sandwiched between the arms 1502A, 1502B of head 1510A. In other examples, as shown by FIG. 18C, both the SHM 254 and the STM 244 are targeted by head 1510A using arms 1502A, 1502B with arm 1502A being between STM 244 and SHM 254, and arm 1502B being anterior to the SHM 254.

With this in mind, as shown by FIG. 18B, in some examples, in some examples, the heads 1510A, 1510B of the stimulation element 1507 may be positioned at, near, and/or at least partially around the STM 244 and the SHM 254 on one or both sides of the patient such that the stimulation electrodes 1506 arranged on the heads 1510A, 1510B of the stimulation element 1507 are in stimulating relation to the STM(s) 244 and the SHM(s) 254, and/or to the IHM-innervating nerve. For example, at least a portion of the stimulation electrodes 1506A, 1506B, 1506C, 1506D may be operated to apply a stimulation vector (e.g., via electrode pairs) across and through tissue, such as through the STM 244 and the SHM 254 to capture at least the STM 244 and/or the IHM-innervating nerve. It will be understood that, via such example arrangements, the SHM 254 also may become captured via the electrical stimulation. However, it is believed that in at least some instances, such electrical stimulation of the SHM 254 would not significantly hamper the intended effect to be achieved via the electrical stimulation of the STM 244 which occurs even while the SHM 254 receives some electrical stimulation.

In some examples, the heads 1510A, 1510B may be positioned at a location (e.g., along a generally superior-inferior orientation) where the STM 244 and the SHM 254 cross one another. As shown by FIG. 18B, arms 1502A may be placed posterior to (e.g., behind) the STMs 244 and arms 1502B may be placed anterior to (e.g., in front of) the SHMs 254.

In some examples, as shown by FIG. 18C, arms 1502A may be placed between the STMs 244 and SHMs 254, and arms 1502B may be placed anterior to the SHMs 254. In some such examples (or any of the examples described herein), arms 1502A may include stimulation electrodes 1506A, 1506B, 1506D, 1506E on both sides of the arms 1502A, with stimulation electrodes 1506A, 1506D facing the SHMs 254 and stimulation electrodes 1506B, 1506) facing the STMs 244. Arms 1502B may include stimulation electrodes 1506C, 1506F on the inside of the arms 1502B which face the SHMs 254. In some examples, both arms 1502A, 1502B may have stimulation electrodes on both sides of the arms 1502A, 1502B.

In any of the deployments 1501, 1505, 1513, the stimulation element 1507 may include a fixation arrangement. For example, on a distal end of the stimulation element 1507, the arms 1502A, 1502B of the U-shaped heads 1510A, 1510B may include a fixation arrangement on or attached thereto, such as including tines, barbs, ridges and/or other tissue-engaging structures to hinder or prevent movement of the stimulation element 1507. In some examples, the arms 1502A, 1502B of the heads 1510A, 1510B may be coupled to a fixation arrangement as previously described in connection with FIGS. 14A-14B, where the catch structure is inserted through a portion of the SHM 254, the STM 244, or other proximate tissue.

In some examples, and using any of the above-described deployments 1501, 1503, 1513, the stimulation element 1507 may be shaped, positioned, and/or include fixation arrangements to provide support and/or to limit movement once the stimulation element 1507 is deployed. In some examples, such as those illustrated by FIGS. 18A and 18C, at least one arm 1502A, 1502B of the heads 1510A, 1510B may be sandwiched between the STM 244 and SHM 254, such that the respective arm 1502A, 1502B is supported and/or movement is limited by the SHM 254 and/or STM 244. In this manner, the shape, size, and/or orientation of the heads 1510A, 1510B may permit placement in and among muscles in a way that the size, shape, and/or orientation of the arms 1502A, 1502B contribute to securely fixing the heads 1510A, 1510B, and thus the stimulation electrodes 1506A, 1506B, 1506C, 1506D (and optionally 1506E, 1506F), relative to the STMs 244 and SHMs 254. In some examples, which may be in addition to the above and are further described below, the heads 1510A, 1510B may include fixation arrangements to provide support and/or limit movement, such as fixation elements on surfaces of the arms 1502A, 1502B. For example, surfaces of the arms 1502A, 1502B which are in contact with or near tissue of the STM 244 and/or SHM 254 may include fixation elements such as sutures, tines, barbs, ridges and/or other tissue-engaging structures to hinder or prevent movement of the stimulation element 1507. In further examples, one or both of the arms 1502A, 1502B of the heads 1510A, 1510B may be inserted into the SHM 254 and/or STM 244 to hinder or prevent movement of the stimulation element 1507. In some examples, the stimulation element 1507 further includes a lead 1511 coupled to the U-shaped head(s) 1510A, 1510B (e.g., to base 1503) at a first (e.g., distal) end of the lead 1511 and coupled to an IPG 1512 at an opposite second (e.g., proximal) end of the lead 1511. The lead 1511 may include lead body 1509 and a bifurcation portion 1504 from which two flexible distal lead segments 1503A, 1503B extend to the heads 1510A, 1510B. In some examples, the IPG 1512 may be implanted in a pectoral region or attached to other tissue, such as a clavicle of the patient. In some examples, tissues superior and/or posterior to the implanted position of heads 1510A, 1510B may be anchoring tissues to which the IPG 1512 may be engaged. In some examples, such as further described in connection with at least FIG. 23, the pulse generator (PG) and/or other functionalities described herein may be located external to the patient's body. In some such examples, the entire PG (and/or other power, control, and/or communication elements) may be implantable while in some examples, some portions of the PG (and/or other power, control, and/or communication elements) may be external to the patient. Similarly, the IPGs 1512 may be implemented as described in connection with any of FIGS. 4-5E.

In some examples, anchoring the stimulation element 1507 on the distal end (e.g., via heads 1510A, 1510B) and the proximal end (e.g., IPG 1512 or a fixation arrangement) may prevent or mitigate movement of the stimulation element 1507 once implanted and/or while applying stimulation. In some examples, at least a portion of the lead body 1509 may comprise part of a fixation arrangement for the stimulation element 1507, with such portion(s) of the lead body 1509 including tines, barbs, ridges, and/or other fixation elements of the type provided in example FIGS. 14A-17EG.

As may be appreciated, examples may include stimulation elements which are deployed on one side of the patient's body but not the other side of the patient's body (e.g., are not bilateral). In such examples, any of the stimulation elements 1507 of FIGS. 18A-18C may include a head 1510A on the one side (e.g., right) of the patient's body but not the other side (e.g., left) of the patient's body. In some examples, the example arrangements of FIGS. 1A-18C may be implemented to apply electrical simulation to the STM 244 and/or SHM 254.

In some examples, any of the stimulation elements 1507 illustrated by FIGS. 18A-18C may be deployed such that at least one arm 1502A, 1502B of the heads 1510A, 1510B may be inserted into the STM 244 and/or into SHM 254. In some examples, one of the arms 1502A, 1502B may be inserted into the STM 244 and the other of the arms 1502A, 1502B may be inserted into the SHM 254. In some examples, one of the arms 1502A, 1502B is inserted into the STM 244 or into the SHM 254 and the other of the arms 1502A, 1502B is not inserted into either the STM 244 and SHM 254. In some such examples, the STM 244 and/or SHM 254 may be activated via electrodes on the arms 1502A, 1502B without use of a vector.

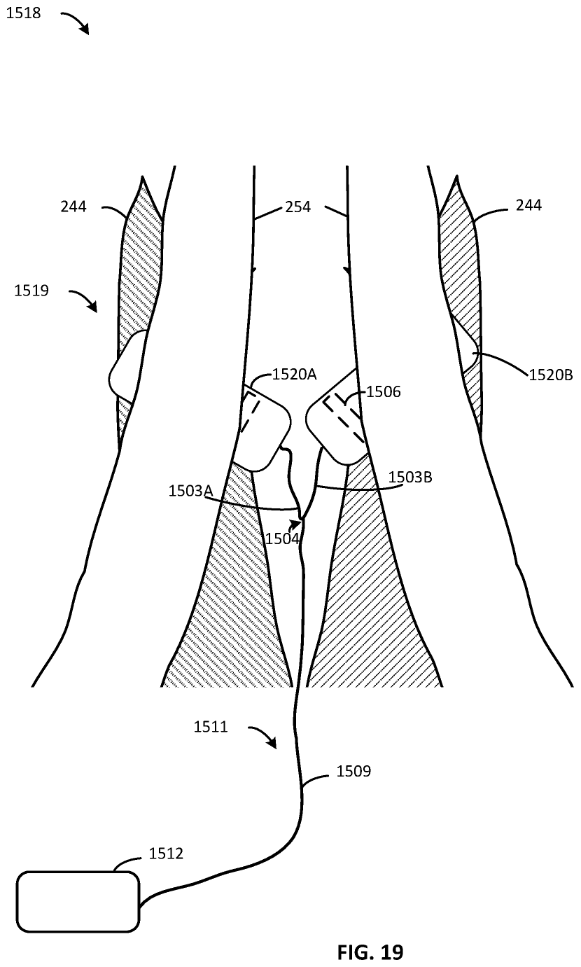

FIG. 19 illustrates an example deployment 1518 of a stimulation element 1519 comprising a pair of paddle-like bodies 1520A, 1520B. In some examples, the stimulation element 1519 may include an implementation of and/or include at least some of substantially the same features and attributes as the stimulation elements and paddle-like bodies 954 of FIGS. 11A-12B and/or 16A-17C. The common features and attributes are not repeated for clarity.

As shown in FIG. 19, the stimulation element 1519 may be implanted at or near IHMs 244, 254 in a head-and-neck region of the patient. For example, the paddle-like bodies 1520A, 1520B may be placed between the STM 244 and the SHM 254, such as being posterior to the SHM 254 and anterior to the STM 244, and at a point where the STM 244 and SHM 254 cross. In some examples, the paddle-like bodies 1520A, 1520B may be placed posterior to the STM 244 or anterior to the SHM 254.

In some examples, the paddle-like bodies 1520A, 1520B may include electrodes 1506 on both sides of the paddle-like bodies 1520A, 1520B. In some such examples, the stimulation electrodes 1506 on both sides of the bodies 1520A, 1520B may be independently addressable in order to selectively apply stimulation to different tissue, such as to the STM 244, to the SHM 254, or to both the STM 244 and SHM 254. In other examples, the paddle-like bodies 1520A, 1520B may include electrodes 1506 on one side of the bodies 1520A, 1520B, with the stimulation electrode 1506 being placed at or near the infrahyoid muscle to be stimulated and may be independently addressable. For example, the paddle-like bodies 1520A, 1520B may be placed so that the electrodes 1506 are facing the STM 244.

In some examples, the stimulation element 1519 may include at least one fixation element, with the stimulation element 1519 and fixation element(s) forming a fixation arrangement. For example, on a distal end of the stimulation element 1519, the paddle-like bodies 1520A, 1520B may include a fixation element(s) on or attached thereto, such as sutures, tines, barbs, ridges and/or other tissue-engaging structures to hinder or prevent movement of the stimulation element 1507, such as those illustrated by FIGS. 14A-16C. In some examples, the stimulation element 1519 further includes a lead 1511 coupled to the bodies 1520A, 1520B at the first end of the lead 1511 and coupled to an IPG 1512 at the opposite second end of the lead 1511, as previously described in connection with FIGS. 18A-18C. The common features are not repeated for ease of reference.

As may be appreciated, examples may include stimulation elements which are deployed on one side and not the other (e.g., are not bilateral). In such examples, the stimulation element 1519 of FIG. 19 may include a paddle-like body 1520A on the one side (e.g., right) and not the other (e.g., left).

FIGS. 20A-20E illustrate example deployments (e.g., 1530A, 1530B, 1530C, 1537) of stimulation elements 1531 comprising an array of spaced out stimulation electrodes 852A, 852B, 852C, 852D, 852E, 852F, 852G, 852H, 8521, 852J (herein generally referred to as “stimulation electrodes 852”) supported on a flexible lead portion 850. In some examples, each stimulation element 1531 of FIGS. 20A and 20C-20D may include an implementation of at least some aspects of, and/or include at least some of substantially the same features and attributes as, the stimulation element 853 of FIG. 10. The common features and attributes are not repeated for clarity.

As shown by FIG. 20A, the stimulation element 1531 may be placed (e.g., within subcutaneous tissue) to extend along and around at least portions of the IHMs 244, 254. For example, the flexible lead portion 850 may be placed anterior to the SHM 254, between the SHM 254 and the STM 244, and posterior to the STM 244 such that respective stimulation electrodes 852 are positioned around portions of the SHM 254 and STM 244.

In some examples, the stimulation electrodes 852 are positioned to generate different stimulation vectors between various combinations of the electrodes 852. As an example, stimulation vector V1 may be applied relative to the STM 244 via electrodes 852F, 8521 and stimulation vector V2 may be applied relative to SHM V2 via electrodes 852A, 852E. As may be appreciated, additional stimulation vectors may be applied via selection of different electrode pairs and/or may be used to capture other tissue, such as the IHM-innervating nerve. It will be understood that the particular depicted stimulation vectors V1, V2 are merely examples that stimulation vectors between combinations of the stimulation electrodes 852.

In some examples, the stimulation element 1531 may be delivered and/or at least partially secured via at least one fixation arrangement. FIG. 20B illustrates an example fixation arrangement 1532 comprising a tether 1314 and catch structure 1312, and which can include an implementation of the flexible attachment device 1302 of FIG. 14A in some examples. In some examples, the flexible attachment device (e.g., tether 1314) may be used as a guidewire to first establish a pathway within tissue prior to, and for, delivering a lead (e.g., 1540) along the pathway. In some such examples, the tether 1314 acting as a guidewire is chronically implanted and becomes part of the fixation arrangement 1532 for the stimulation element 1531 (FIG. 20A). In some such examples, the stimulation element 1531 includes a fixation arrangement, which may comprise the fixation arrangement 1532 and/or other fixation arrangements. The common features are not repeated for ease of reference.

In the examples, the stimulation element 1531 is deployed as described above in connection with FIG. 20A. In some examples, the stimulation element 1531 is deployed as illustrated by the series of FIGS. 20B-20D. For example, the flexible attachment device is deployed as illustrated and described above in connection with FIG. 14A and FIG. 20B. In some examples, the distal end of the flexible attachment device includes a tether 1314 coupled to a catch structure 1312. As described in connection with FIGS. 14A-14B, the catch structure 1312 may be moveable and placed through tissue 1301 using a needle, such that the catch structure 1312 is on a first side of fixation tissue 1301 and the remaining portion of the tether 1314 is on a second opposite side of the fixation tissue 1301.

In some examples, as shown by FIG. 20C, a distal portion 1541A of a lead body 1540 of the flexible lead 850 (which has a lumen extending therethrough to receive the tether 1314 acting as a guidewire) may be placed over the tether 1314 at a proximal end 1317B of the tether 1314, and then the distal portion 1541A and the rest of lead body 1540 may be advanced (as represented via directional arrow ADV) toward the fixation tissue 1301 at a distal end 1317A of the tether 1314, as shown by FIG. 20D. In some examples, as further shown by FIG. 20D, a suture anchor 1542 (or other fixation element) is used to secure the lead body 1540 to surrounding non-nerve tissue, which in turn acts to secure the stimulation element 1531 (including the electrodes 852) relative to the patient's body and in stimulating relation relative to target tissues. At least some examples of fixation element include sutures, tines, barbs, ridges and/or other tissue-engaging structures to hinder or prevent movement of the stimulation element 1531, such as those previously described in connection with at least FIGS. 15A-17C and/or other examples of fixation arrangements in various examples of the present disclosure.

FIG. 20E illustrates an example deployment 1537 of the stimulation element 1531 of FIGS. 20A-20D, with the stimulation element 1531 bilaterally placed across both sides of the patient. As may be appreciated, the lead portion 850 may be placed anteriorly to each SHM 254, between each SHM 254 and STM 244, and posteriorly to each STM 244, similar to that described above in connection with FIG. 20A but with the lead portion 850 extending across the midline of the patient. In some examples, the lead portion 850 may be between each of the SHM 254 and STM 244, and posteriorly to each STM 244, with the STM 244 being the primary stimulation target. In some examples, the stimulation element 1531 may be deployed on one side of the patient and not the other.

In some examples, the stimulation element 1531 further includes an IPG 1512 at the second (e.g., proximal) end of the lead portion 850, as previously described in connection with FIGS. 18A-18C. The common features are not repeated for ease of reference.

In some examples, the example deployment 1537 may be implemented via at least some of the features of the example of FIGS. 20B-20D.

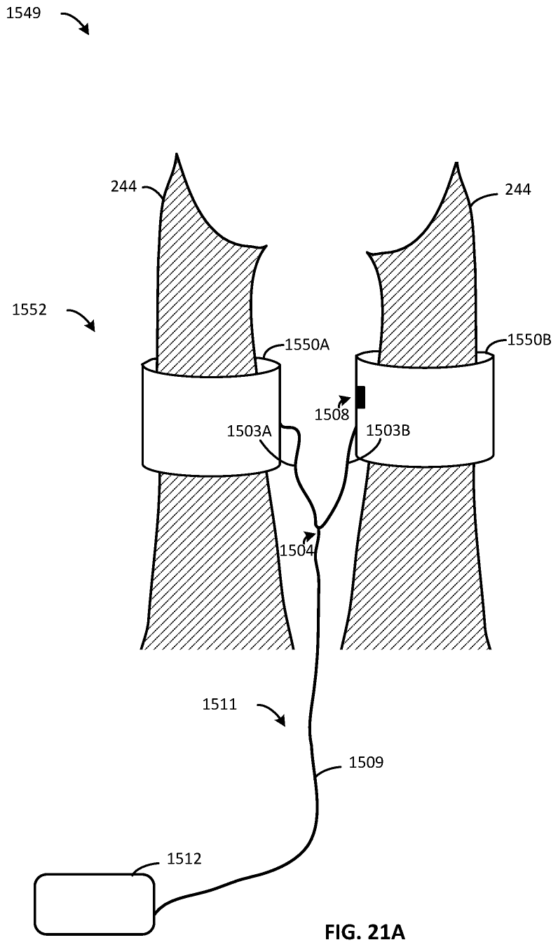

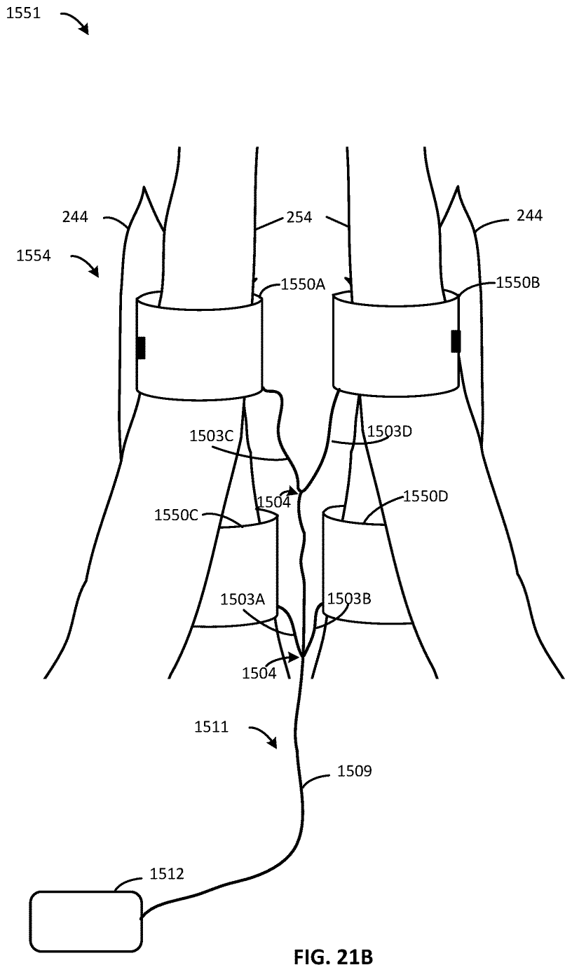

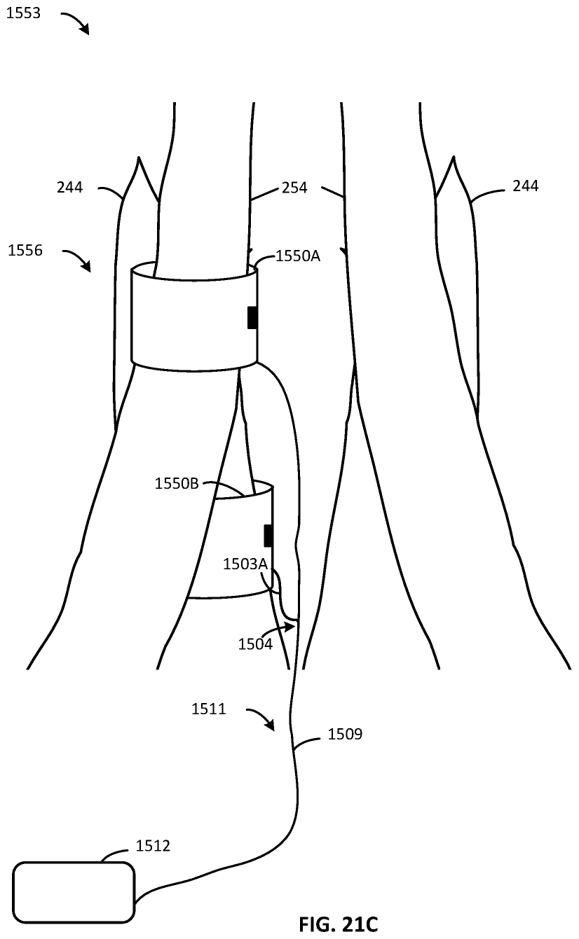

FIGS. 21A-21C illustrate example deployments 1549, 1551, 1553 of stimulation elements 1552, 1554, 1556 comprising at least one electrode cuff 1550A, 1550B, 1550C, 1550D. In some examples, each of the stimulation elements 1552, 1554, 1556 of FIGS. 21A-21C may include an implementation of and/or include at least some of substantially the same features and attributes as the stimulation elements 1100 of FIG. The common features and attributes are not repeated for clarity.

As shown by FIG. 21A, in some examples, the stimulation element 1552 comprises a pair of electrode cuffs 1550A, 1550B which are positioned to at least partially surround the STM 244 on one or both sides of the patient such that the stimulation electrodes arranged on electrode cuffs 1550A, 1550B are in stimulating relation to the STM(s) 244 and/or to the IHM-innervating nerve. For example, at least a portion of the stimulation electrodes (not shown) may be operated to apply a stimulation vector across and through tissue, such as through the STM 244 and to capture the STM 244 and/or the IHM-innervating nerve. In some examples, each electrode cuff 1550A, 1550B may be independently addressable, such that the STM 244 on the left side and the STM 244 on the right side of the patient, and/or IHM-innervating nerve, may be selectively stimulated.

In some examples, at least one of the electrode cuffs 1550A, 1550B (and/or electrode cuffs 1550A-1550D illustrated by FIGS. 21B and/or 1550A, 1550B illustrated by FIG. 21C) may include at least one sensing electrode 1508 on the exterior surface of the cuff body or arm of the electrode cuff 1550A, 1150B. The sensing electrode 1508 may be used to sense physiologic signals from a tissue (e.g., nerve and/or muscle) and which may be used to identify respiratory information, such as further described herein in connection with sensing element 2000 of FIG. 25. Among other aspects, placing sensing electrode(s) 1508 on an exterior surface of the cuff helps to isolate sensing from the stimulation signal applied via the stimulation electrodes exposed on an interior surface/wall of the cuff 1550A, 1550B, thereby increasing the accuracy and/or effectiveness of such sensing.

In some examples, both the STM 244 and the SHM 254 may be captured by a stimulation element. For example, as shown by FIG. 21B, in some examples, the stimulation element 1554 comprises two pairs of electrode cuffs 1550A, 1550B and 1550C, 1550D which are positioned to at least partially surround the SHM 254 and the STM 244 on both sides of the patient such that the stimulation electrodes (not shown) arranged on the electrode cuffs 1550A, 1550B, 1550C, 1550D are in stimulating relation to the STM 244 and the SHMs 254 and/or to the IHM-innervating nerve. In particular, on one side of the body, cuff 1550A at least partially encircles SHM 254 and cuff 1550C at least partially encircles STM 244, while on the other side of the body, cuff 1550B at least partially encircles SHM 254 and cuff 1550D at least partially encircles STM 244. For example, at least a portion of the stimulation electrodes may be operated to apply a stimulation vector across and through tissue, such as through the STM 244 and/or through the SHM 254 and to capture the STM 244, the SHM 254, and/or the IHM-innervating nerve. In some examples, each electrode cuff 1550A, 1550B, 1550C, 1550D and/or the respective stimulation electrodes may be independently addressable, such that the STM 244 and/or SHM 254 on the left side and the STM 244 and/or SHM 254 on the right side of the patient, and/or IHM-innervating nerve, may be selectively stimulated.

As may be appreciated, examples may include stimulation elements which are deployed on one side and not the other of the patient(e.g., are not bilateral). In such examples, any of the stimulation elements 1552, 1554, 1556 of FIGS. 21A-21B may include electrode cuffs on the one side (e.g., left) and not the other (e.g., right). For example, as shown by FIG. 21C, the stimulation element 1556 includes a pair of electrode cuffs 1550A, 1550B which are positioned to at least partially surround the SHM 254 and the STM 244, respectively on one side of the patient such that the stimulation electrodes (not shown) arranged on the of electrode cuffs 1550A, 1550B are in stimulating relation to the STM 244 and the SHM 254 and/or to the IHM-innervating nerve.

In some examples, any of the stimulation elements 1552, 1554, 1556 may include at least one fixation arrangement, such as sutures, tines, barbs, ridges and/or other tissue-engaging structures to hinder or prevent movement of the stimulation element 1552, 1554, 1556, such as those previously described in connection with FIGS. 14A-16E. In some examples, the stimulation element 1552, 1554, 1556 further includes a lead 1511 coupled to the bodies 1520A, 1520B at the first end of the lead 1511 and coupled to an IPG 1512 at the opposite second end of the lead 1511, as previously described in connection with FIGS. 18A-18C. The common features are not repeated for ease of reference.

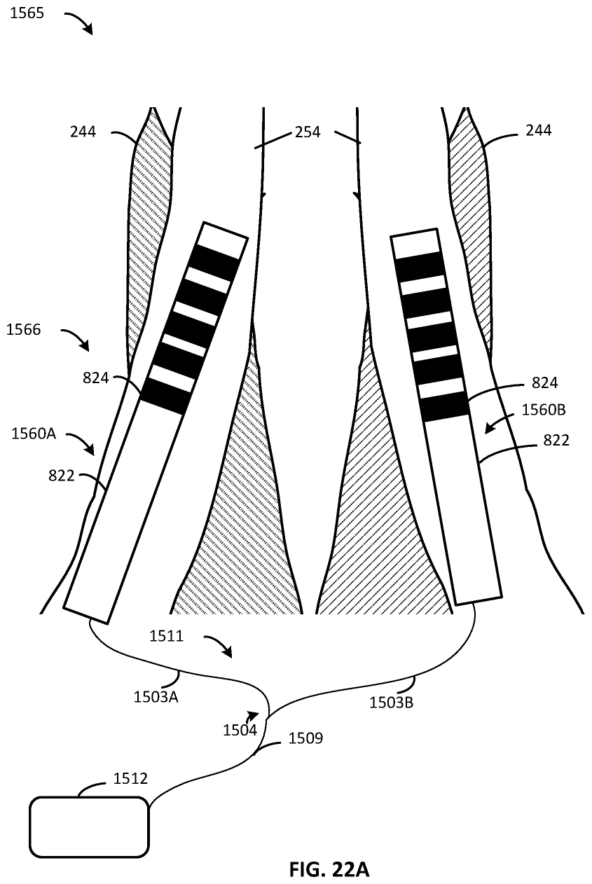

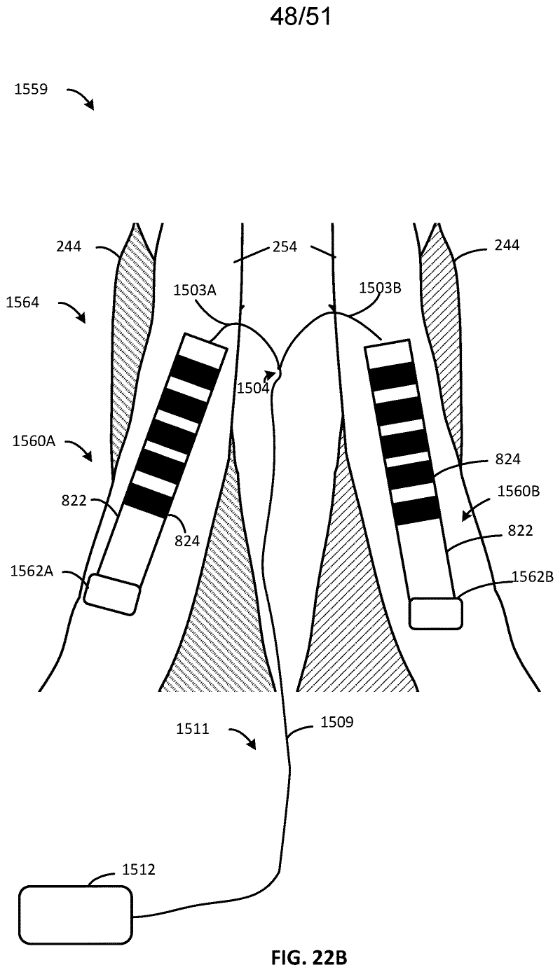

FIGS. 22A-22B illustrate example deployments 1565, 1559 of stimulation elements 1564, 1566 comprising a stimulation portion 1560A, 1560B that includes at least one axial array of electrodes 824 supported on a lead body 822. In some examples, each of the stimulation elements 1564, 1566 of FIGS. 22A-22B may include an implementation of and/or include at least some of substantially the same features and attributes as the stimulation elements 700, 820 of FIGS. 8A-9C. The common features and attributes are not repeated for clarity.

As shown in FIG. 22A, the stimulation element 1566 may be implanted near IHMs 244, 254 in a head-and-neck region of the patient. For example, the stimulation portions 1560A, 1560B may be placed between the STM 244 and the SHM 254, such as being posterior to the SHM 254 and anterior to the STM 244, and at a point where the STM 244 and SHM 254 cross. In some examples, the stimulation portions 1560A, 1560B may be placed posterior to the STM 244 or, in some examples, placed anterior to the SHM 254, which is illustrated by FIG. 22A.

In some examples, the electrodes 824 may be ring-like electrodes, ring segments, or non-circular (e.g., linear). In some examples, the electrodes 824 be independently addressable in order to selectively apply stimulation to different tissue, such as to the STM 244, the SHM 254, or both the STM 244 and SHM 254. In some examples, the STM 244 may be selectively stimulated, and in other examples, the STM 244 and SHM 254 may be stimulated.

In some examples, the stimulation element 1566 may include at least one fixation arrangement. For example, FIG. 22B illustrates a stimulation element 1564 which includes an implementation of the stimulation element 1566 of FIG. 22A, but with a fixation arrangement 1562A, 1562B at the first ends of the lead bodies 822. The fixation arrangements 1562A, 1562B may include fixation elements, such as sutures, tines, barbs, ridges and/or other tissue-engaging structures to hinder or prevent movement of the stimulation element 1507, such as those previously described in connection with FIGS. 14A-17EG.

In some examples, the stimulation elements 1564, 1566 further include a lead portion 1511 coupled to the lead body 822 and coupled to an IPG 1512 at the second end of the lead 1511, as previously described in connection with FIGS. 18A-18C. The common features are not repeated for ease of reference.

As may be appreciated, examples may include stimulation elements which are deployed on one side and not the other (e.g., are not bilateral). In such examples, any of the stimulation elements 1564, 1566 of FIGS. 22A-22B may include an array of electrodes 824 on the one side (e.g., right) and not the other (e.g., left).

In any of the above-described examples, the stimulation electrodes may be independently addressable to selectively apply stimulation. For example, the stimulation may be applied via various combinations of the implanted stimulation elements to achieve desired stimulation vectors. The stimulation may be applied among the different stimulation elements in different manners, such as (but not limited to) sequentially, simultaneously, alternating, bilaterally, unilaterally, and/or via other patterns. Further, the particular arrangement (e.g., number, shape, spacing, orientation, etc.) of electrodes may be different from the particular arrangement of electrodes illustrated by FIGS. 7A-22B.

As may be appreciated and in some examples, portions of stimulation elements are shown in solid lines at locations at which the portions of the stimulation elements are posterior (e.g., behind) tissue or other structures of the stimulation element. Such portions are illustrated in solid lines for simplicity purposes. Similar representations are made throughout the disclosure.

FIG. 23 is a diagram including a front view schematically representing a patient's body, implantable components, and/or external elements of example methods and/or example devices. More specifically, FIG. 23 is a block diagram representing a patient's body 1640, including example target portions 1641-1664 at which at least some example sensing element(s) and/or stimulation elements may be employed to implement at least some examples of the present disclosure.

As shown in FIG. 23, patient's body 1640 comprises a head-and-neck portion 1641, including head 1642 and neck 1644. Head 1641 comprises cranial tissue, nerves, etc., and upper airway 1646 (e.g., nerves, muscles, tissues), etc. As further shown in FIG. 23, the patient's body 1640 comprises a torso 1650, which comprises various organs, muscles, nerves, other tissues, such as but not limited to those in pectoral region 1652 (e.g., lungs 1653, cardiac 1657), abdomen 1654, and/or pelvic region 1656 (e.g., urinary/bladder, anal, reproductive, etc.). As further shown in FIG. 23, the patient's body 1640 comprises limbs 1660, such as arms 1662 and legs 1664.

It will be understood that various sensing elements (and/or stimulation elements) as described throughout the various examples of the present disclosure may be deployed within the various regions of the patient's body 1640 to sense and/or otherwise diagnose, monitor, treat various physiologic conditions such as, but not limited to those examples described in association with FIGS. 1A-22B. In some such examples, a stimulation element 1647 may be located in or near the upper airway 1646 for treating sleep disordered breathing (and/or near other nerves/muscles for treating other conditions) and/or a sensing element 1658 may be located anywhere within the neck 1644 and/or torso 1650 (or other body regions) to sense physiologic information for providing patient care (e.g., SDB, other) with the sensed physiologic information.

In some examples, the stimulation element 1647 may comprise at least some of substantially the same features and attributes as the various stimulation elements described throughout the various examples of the present disclosure in association with at least FIGS. 4-22B.

Further details regarding a location, structure, operation, and/or use of the sensing element 1658, external element(s) 1670, and/or stimulation element 1647 are described in association with at least FIGS. 1F-1G and FIGS. 4-22B.

In some examples, at least a portion of the stimulation element 1947 may comprise part of an external component/device. In some examples, a portion of the stimulation element 1647 may be implantable and a portion of the stimulation element 1647 may be external to the patient. Accordingly, as further shown in FIG. 23, the various sensing element(s) 1658 and/or stimulation element(s) 1647 implanted in the patient's body may be in wireless communication (e.g., connection 1667) with at least one external element 1670.

As further shown in FIG. 23, in some examples, the external element(s) 1670 may be implemented via a wide variety of formats such as, but not limited to, at least one of the formats 1671 including a patient support 1672 (e.g., bed, chair, sleep mat, other), wearable elements 1674 (e.g., finger, wrist, head, neck, shirt), noncontact elements 1676 (e.g., watch, camera, mobile device, other), and/or other elements 1678.

As further shown in FIG. 23, in some examples, the external element(s) 1670 may comprise at least one of the different modalities 1680 such as (but not limited to) a sensing portion 1681, stimulation portion 1682, power portion 1684, communication portion 1986, and/or other portion 1988. The different portions 1681, 1682, 1684, 1686, 1688 may be combined into a single physical structure (e.g., package, arrangement, assembly), may be implemented in multiple different physical structures, and/or with just some of the different portions 1681, 1682, 1684, 1686, 1688 combined together in a single physical structure.

Among other such details, in some examples the external sensing portion 1681 and/or implanted sensing element 1658 may comprise at least some of substantially the same features and attributes of at least sensing portion 2000, as further described below in FIG. 24.

In some examples, the stimulation portion 1682 and/or implanted stimulation element 1647 may comprise at least some of substantially the same features and attributes of at least the stimulation arrangements, as further described below in association with at least FIG. 24 and/or other examples throughout the present disclosure such as FIGS. 1A-22B and 25-29.

In some examples, the external power portion 1684 and/or power components associated with implanted stimulation element 1647 may comprise an example implementation of, and/or at least some of substantially the same features and attributes as, at least the stimulation arrangements, as further described in association with at least FIGS. 1A-22B and/or other examples throughout the present disclosure. In some such examples, the respective power portion, components, etc. may comprise a rechargeable power element (e.g., supply, battery, circuitry elements) and/or non-rechargeable power elements (e.g., battery). In some examples, the external power portion 1684 may comprise a power source by which a power component of the implanted stimulation element 1947 may be recharged.

In some examples, the wireless communication portion 1686 (e.g., supporting and/or including connection/link at 1667) may be implemented via various forms of radiofrequency communication and/or other forms of wireless communication, such as (but not limited to) magnetic induction telemetry, BT, BLE, NIF, near-field protocols, Wi-Fi, Ultra-Wideband (UWB), and/or other short range or long range wireless communication protocols suitable for use in communicating between implanted components and external components in a medical device environment.

Examples are not so limited as expressed by other portion 1688 via which other aspects of implementing medical care may be embodied in external element(s) 1670 to relate to the various implanted and/or external components described above.

FIG. 24 is a schematic diagram of a control portion, which may comprise at least some of substantially the same features and attributes as the control portion 2100 of FIG. 27A. Among other aspects, example methods and/or example devices may be implemented via the control portion 1690. In some examples, the control portion 1690 may be used to implement at least some of the various example devices and/or example methods of the present disclosure as described herein. In some examples, the control portion 1690 may form part of, and/or be in communication with, the stimulation element (e.g., 1647 in FIG. 23), sensing element 1658, and/or other medical device.

FIG. 25 is a block diagram schematically representing an example sensing portion of an example device and/or used as part of example method. In some examples, an example method may employ and/or an example SDB care device (e.g., including stimulation element 110 in FIG. 1F) may comprise the sensing portion 2000 to sense physiologic information and/or other information, with such sensed information relating to care of a wide variety of physical conditions such as, but not limited to, sleep disordered breathing care, pelvic care, cardiac care, among other uses.

The sensed information may be used to implement at least some of the example methods and/or examples devices described in association with at least FIGS. 1A-24 and/or FIG. 26. It will be understood that the sensing portion 2000 may be implemented as single sensor or multiple sensors, and may comprise a single type of sensor or multiple types of sensing. In addition, it will be further understood that the various types of sensing schematically represented in FIG. 25 may correspond to a sensor and/or a sensing modality.

In some examples, the sensed information may refer to physiologic signals (e.g., biosignals) and/or metrics which may derived from such physiologic signals. For example, among other sensed physiologic signals, one physiologic signal may comprise respiration (parameter 2005 in FIG. 25), from which various metrics may be derived such as, but not limited to, respiratory rate, respiratory rate variability, respiratory phase, rate times volume, waveform morphology, and more. The respiration information may be sensed via at least one of the sensing modalities described below (and/or other sensing modalities) such as, but not limited to, accelerometer 2026, ECG 2020, impedance 2036, pressure 2037, temperature 2038, acoustic 2039, and/or other sensing modalities, at least some of which are further described below. The respiration information may be used for a wide variety of purposes such as, but not limited to, timing stimulation relative to respiration, disease burden, sleep-wake status, arousals, etc. In some such examples, the detection of disease burden may comprise detection of sleep disordered breathing events, which may be used in determining, assessing, etc. therapy outcomes such as, but not limited to, AHI.

In some examples, the sensed physiologic information may comprise cardiac information (2006) obtained from a cardiac signal and from which various metrics may be derived such as, but not limited to, heart rate (HR), heart rate variability (HRV), P-R intervals, waveform morphology, and more. One example of a cardiac signal may comprise an ECG signal, as represented at 2020 in FIG. 25. Accordingly, the cardiac information and/or signal may be sensed via at least one sensing modality further described below (and/or other sensing modalities) such as, but not limited to, cardiac sensor 2023, accelerometer 2026, ECG 2020, EMG 2022, impedance 2036, pressure 2037, temperature 2038, and/or acoustic 2039. In some examples, the sensed physiologic information (e.g., via sensing portion 2000) may comprise a wide variety of physiologic information other (2007) than respiration and/or cardiac information, with at least some examples described throughout the present disclosure.

The sensed physiologic signals and/or information (e.g., respiration 2005, cardiac 2006, and/or other information 2007) may be used for a wide variety of purposes such as, but not limited to, determining sleep-wake status (e.g., various sleep onset determinations), timing stimulation relative to respiration, determining disease burden, determining arousals, etc. In some such examples, the determination of disease burden may comprise detection of sleep disordered breathing events, which may be used in determining, assessing, etc. therapy outcomes such as, but not limited to, AHI, as well as titrating stimulation parameters, adjusting sensitivity of sensing the physiologic information, etc.

For instance, in one non-limiting example, an ECG sensor 2020 in FIG. 25 may comprise a sensing element (e.g., electrode) or multiple sensing elements arranged relative to a patient's body (e.g., implanted in the transthoracic region) to obtain ECG information. In some examples, the ECG information may comprise one example implementation to obtain cardiac information, including but not limited to, HR 2025A (HR), HRV 2025B, and other cardiac parameters 2025C, which may be used (with or without other information) in determining delivering stimulation therapy and associated sensing (e.g., inputs) for determining effectiveness of the therapy and/or implementing the therapy, as described throughout the examples of the present disclosure.

However, in some instances, the ECG sensor 2020 may represent ECG sensing element(s) in general terms without regard to a particular manner in which sensing ECG information may be implemented.

In some examples in which multiple electrodes are employed to obtain an ECG signal, an ECG electrode may be mounted on or form at least part of a case (e.g., outer housing) of a stimulation support portion (which may comprise an IPG in some examples). In such instances, other ECG electrodes are spaced apart from the ECG electrode associated with the stimulation support portion. In some examples, at least some ECG sensing electrodes also may be employed to deliver stimulation to a nerve or muscle, such as but not limited to, an upper airway patency-related nerve (e.g., hypoglossal nerve) or other nerves or muscles.

In some examples, other types of sensing may be employed to obtain cardiac information (including but not limited to heart rate and/or heart rate variability), such as a cardiac sensor 2023 shown in FIG. 25, which may comprise at least one of a ballistocardiogram sensor(s), seismocardiogram sensor(s), and/or accelerocardiogram sensor(s). In some examples, such sensing is based on and/or implemented via accelerometer-based sensing such as further described below in association with accelerometer 2026.

In one aspect, in some examples in which the cardiac sensor 2023 comprises a ballistocardiogram sensor, the sensor senses cardiac information caused by cardiac output, such as the forceful ejection of blood from the heart into the great arteries that occurs with each heartbeat. The sensed ballistocardiogram information may comprise HR 2025A, HRV 2025B, and/or additional cardiac morphology 2025C. In some examples such ballistocardiogram-type information may be sensed from within a blood vessel in which the sensor (e.g., accelerometer) senses the movement of the vessel wall caused by pulsations of blood moving through the vessel with each heartbeat. This phenomenon may sometimes be referred to as arterial motion.

In some examples in which the cardiac sensor 2023 comprises a seismocardiogram sensor, the sensor 2023 may provide cardiac information which is similar to that described for ballistocardiogram sensor, except for being obtained via sensing vibrations, per an accelerometer (e.g., single or multi-axis), in or along the chest wall caused by cardiac output. In particular, the seismocardiogram measures the compression waves generated by the heart (e.g., per heart wall motion and/or blood flow) during its movement and transmitted to the chest wall. Accordingly, the sensor 2023 may be placed in the chest wall.

In some such examples of sensing per sensor 2023, such methods and/or devices also may comprise sensing a respiratory rate and/or other respiratory information.

In some examples the sensing portion 2000 may comprise an electroencephalography (EEG) sensor 2012 to obtain and track EEG information. In some examples, the EEG sensor 2012 may also sense and/or track central nervous system (CNS) information in addition to sensing EEG information. In some examples, the EEG sensor(s) 2012 may be implanted subdermally under the scalp or may be implanted in a head-and-neck region otherwise suitable to sense EEG information. Accordingly, the EEG sensor(s) 210 are located near the brain and may detect frequencies associated with electrical brain activity.

In some examples, a sensing element used to sense EEG information is chronically implantable, such as in a subdermal location (e.g., subcutaneous location external to the cranium skull), rather than an intracranial position (e.g., interior to the cranium skull). In some examples, the EEG sensing element is placed and/or designed to sense EEG information without stimulating a vagus nerve at least because stimulating the vagal nerve may exacerbate sleep apnea, particularly with regard to obstructive sleep apnea. Similarly, the EEG sensing element may be used in a device in which a stimulation element delivers stimulation to a hypoglossal nerve or other upper airway patency-related nerve without stimulating the vagus nerve in order to avoid exacerbating the obstructive sleep apnea.

In some examples, sensed EEG information may be used as part of (or solely in) making a sleep-wake determination, such as sleep onset, and wake onset. Among other uses, this sleep-wake information may help provide overall sleep hours, which may comprise part of therapy outcome, in some examples.

In some examples, sensed EEG information may be used to detect sleep stages during sleep. Among other uses, this sensed sleep stage may help determine an absolute amount or relative amount of deep sleep, REM sleep per night, and/or other sleep metrics. For instance, such information may be used to evaluate whether a particular stimulation solution setting corresponds to a patient's most therapeutic stimulation energy settings/parameters based on (at least or in part) the recognition more deep sleep typically corresponds to the most or more therapeutic stimulation energy settings whereas less deep sleep typically corresponds to lesser therapeutic stimulation energy settings.

In some examples, sensed EEG information may be used to detect arousals, which may comprise one aspect of determining therapy outcome. Among other uses, the detection of more arousals may provide an indication of the patient exhibiting more daytime sleepiness, which in turn may lead to adjustments to stimulation solution settings (e.g., values of stimulation energy parameters) in order to minimize arousals.

In some examples, the above-described aspects regarding the use of sensed EEG information may be combined in whole, or part, to provide an overall sleep efficiency parameter. In some such examples, the sleep efficiency parameter may be based on: 1) sleep duration; 2) sleep depth; and/or 3) events (e.g., number of arousals). In some examples, the sleep efficiency parameter may be compared to a reference sleep efficiency parameter such as (but not limited to): 1) a reference sleep duration (e.g., 8-9 hours); 2) a reference sleep depth (e.g., a minimum duration of deep sleep and REM sleep; and/or 3) few or no arousals.

In some examples the sensing portion 2000 may comprise an electromyogram (EMG) sensor 2022 to obtain and track EMG information. In some examples, the sensed EMG signals may be used to identify sleep, respiratory information (e.g., respiratory phase information) and/or obstructive events. In some examples, the detected EMG information may be used to detect arousals and/or overall patient movement. These examples of determining and/or using sensed EMG information may be used as part of determining patient metrics (e.g., therapy outcome, usage, other) by which stimulation energy parameters may be determined, adjusted, etc. in order to maintain and/or improve those patient metrics according to various examples of the present disclosure.

In some examples, any one or a combination of the various sensing modalities (e.g., EEG, EMG, etc.) described in association with FIG. 24 may be implemented via a single sensing element 2014.

In some examples, the sensing portion 2000 may comprise an accelerometer 2026. In some examples, the accelerometer 2026 and associated sensing (e.g., motion at (or of) the chest, neck, and/or head, respiratory, cardiac, posture, etc.) may be implemented according to at least some of substantially the same features and attributes as described in Dieken et al., ACCELEROMETER-BASED SENSING FOR SLEEP DISORDERED BREATHING (SDB) CARE, published as U.S. 2019-0160282 on May 30, 2019, and PCT Publication WO 2022/020489, published on Jan. 27, 2022, and entitled “DISEASE BURDEN INDICATION”; and PCT Publication No. WO2022/261311, published on Dec. 15, 2022, and entitled “RESPIRATION SENSING”, and which is incorporated by reference herein in its entirety. In some examples, the accelerometer may comprise a single axis accelerometer while in some examples, the accelerometer may comprise a multiple axis accelerometer.

Among other types and/or ways of sensing information, the accelerometer sensor(s) 2026 may be employed to sense or obtain a ballistocardiogram, a seismocardiogram, and/or an accelerocardiogram (see cardiac sensor 2023 and related disclosure), which may be used to sense (at least) HR 2025A and/or HRV 2025B (among other information such as respiratory rate in in some instances), which may in turn may be used as part of determining respiratory information, cardiac information, as described throughout the examples of the present disclosure. In some examples, this sensed information also may be used in determining sleep-wake status.

In some examples, the accelerometer 2026 may be used to sense activity, posture, and/or body position as part of determining a patient metric, the sensed activity, posture, and/or body position may sometimes be at least partially indicative of a sleep-wake status, which may be used as part of automatically initiating, pausing, and/or terminating stimulation therapy.

In some examples, the sensing portion 2000 may comprise an impedance sensor 2036, which may sense transthoracic impedance or other bioimpedance of the patient. In some examples, the impedance sensor 2036 may comprise a plurality of sensing elements (e.g., electrodes) spaced apart from each other across a portion of the patient's body. In some such examples, one of the sensing elements may be mounted on or form part of an outer surface a housing of a stimulation support portion (e.g., 133 in FIG. 1G) or other implantable sensing monitor, while other sensing elements may be located at a spaced distance from the stimulation support portion and/or stimulation electrode arrangement. In at least some such examples, the impedance sensing arrangement integrates all the motion/change of the body (e.g., such as respiratory effort, cardiac motion, etc.) between the sense electrodes (including the case of the IPG when present). Some examples implementations of the impedance measurement circuit will include separate drive and measure electrodes to control for electrode to tissue access impedance at the driving nodes. Such impedance sensing also may be used for other purposes.

In some examples, the sensing portion 2000 may comprise a pressure sensor 2037, which senses respiratory information, such as but not limited to respiratory cyclical information. In some examples, the pressure sensor 2037 may be located in direct or indirect continuity with respiratory organs or airway or tissues supporting the respiratory organs or airway in order to sense respiratory information.

In some examples, one sensing modality within sensing portion 2000 may be at least partially implemented via another sensing modality within sensing portion 2000.

In some examples, sensing portion 2000 may comprise an acoustic sensor 2039 to sense acoustic information, such as but not limited to cardiac information (including heart sounds), respiratory information, snoring, etc.

In some examples, sensing portion 2000 may comprise body motion parameter 2035 by which patient body motion may be detected, tracked, etc. The body motion may be detected, tracked, etc. via a single type of sensor or via multiple types of sensing. For instance, in some examples, body motion may be sensed via accelerometer 2026 and in some examples, body motion may be sensed via EMG 2022 and/or other sensing modalities, as described throughout various examples of the present disclosure.

In some examples, the sensing portion 2000 in FIG. 25 may comprise a body position/posture parameter 2042 and/or body motion parameter 2035 to sense and/or track sensed information regarding posture, which also may comprise sensing of body position, activity, etc. of the patient. This sensed information may be indicative of an awake or sleep state of the patient in some examples. In some such examples, such information may be sensed via accelerometer 2026 as mentioned above, and/or other sensing modalities. In some examples, such posture information (and/or body position, activity) may be used sometimes alone and/or in combination with other sensing information to determine a patient metric. As described elsewhere herein, in some examples posture may be considered as one of several parameters when determining a probability of sleep (or awake). In some such examples, the sleep-wake status may be used to initiate, pause, and/or terminate stimulation therapy within a nightly treatment period.

In addition or alternatively, sensing activity, motion, and/or body position (e.g., posture) may be used to track a relative degree to which a patient is more active or less active during daytime hours, which may comprise one objective measure of therapy outcome because if the patient is sleeping better at night due to a desirable stimulation solution settings (e.g., values of stimulation energy parameters) which better control sleep disordered breathing, the patient may be much more active during daytime (non-sleep) hours as compared to a baseline in which their sleep disordered breathing was poorly controlled (corresponding to inferior stimulation energy settings) or not controlled at all. Similarly, sensing activity and/or motion as described herein also may be used to detect if the patient tends to falls asleep during daytime (e.g., non-sleep) hours, which may be an objective therapy outcome parameter by which stimulation energy parameters (and associated usage, and other therapy outcome parameters) may be evaluated and potentially adjusted according to at least some examples of the present disclosure. This objective therapy outcome information also may be used in conjunction with subjective therapy outcome information such as, but not limited to, the Epworth Sleepiness Scale (ESS) and/or other forms of patient input regarding the patient's perceived daytime sleepiness, daytime functional ability, perceived sleep quality, etc.

In some examples, the sensing portion 2000 may comprise an other parameter 2041 to direct sensing of, and/or receive, track, evaluate, etc. sensed information other than the previously described information sensed via the sensing portion 2000.

As further shown in FIG. 25, in some examples the sensing portion 2000 may comprise a temperature sensor 2038. In some example methods, sensing a change in temperature (such as via sensor 2038) during a treatment period may be used to identify sleep disordered breathing behavior. In some such examples, additional sensed information (as described in examples of the present disclosure) may be used in addition to sensed temperature to identify sleep SDB behavior. In some examples, smaller yet detectable temperature changes within a treatment period may be used to at least partially determine a patient metric. For instance, a detectable temperature change may be sensed as a result of patient exertion to breathe in response to an apnea event, given the greater muscular effort in attempting to breathe.

In some examples, at least some of the sensors and/or sensor modalities described in association with FIG. 25 may be incorporated within or on a stimulation element (e.g., 110 in FIG. 1F) which comprise at least some implantable components, in some examples.

FIG. 26 is a block diagram schematically representing an example stimulation portion. In some examples, the stimulation portion 2200 may comprise an example further implementation of, and/or at least some of substantially the same features and attributes as, the stimulation support portion (e.g., 133 in FIG. 1G) described throughout examples of the present disclosure and/or the control portion (e.g., FIG. 24, FIGS. 27A-29) of the present disclosure. Accordingly, the various functions and parameters of the stimulation portion 2200 may be implemented in a manner supportive of, and/or complementary with, the various functions, parameters, portions, etc. of such examples and/or various functions, parameters, portions, etc. relating to stimulation throughout examples of the present disclosure.

In some examples, via target tissue parameter 2210, stimulation may be delivered to selectable target tissues such as, but not limited to, upper airway patency-related tissues. In some examples, the upper airway patency-related tissue may comprise a hypoglossal nerve and/or muscle (e.g., genioglossus muscle) innervated by the hypoglossal nerve to cause contraction of at least the protrusor muscles to cause protrusion of the tongue to increase and/or maintain upper airway patency. In some examples, the upper airway patency-related tissue may comprise IHM-innervating nerves, as previously described, which innervate at least one IHM (e.g., thyrohyoid, omohyoid, sternohyoid, and/or sternothyroid) and/or at least one IHM. In some examples, target tissues may include any other muscles which affect and/or promote upper airway patency, and/or nerves which innervate such muscles. In some examples, target tissue includes a combination of nerves and/or muscles such as, but not limited to, terminal fiber ends of nerves where a nerve ending terminates into (or at) the muscle being innervated.

In some examples, in addition to or instead of selecting different nerves and/or muscles for stimulation, the target tissue parameter 2210 also may comprise adjusting stimulation parameters via selecting between (or using a combination of) various locations along a nerve such as stimulating multiple different sites along a particular nerve, with some stimulation sites being more distal and some being more proximal.

In some examples, in addition to or instead of selecting different nerves and/or muscles for stimulation, the target tissue parameter 2210 also may comprise adjusting stimulation parameters via selecting between (or using a combination of) different fascicles within a particular nerve in order to selectively stimulate target fibers while omitting (or minimally impacting) stimulation of other nerve fibers.

In some examples, the stimulation portion 2200 may implement stimulation according to a bilateral parameter 2212 in which stimulation is applied to a target tissue on both sides (e.g., left and right) of the patient's body. In some such examples, this bilateral stimulation may be delivered to the same nerve (e.g., hypoglossal nerve) on both sides of the body. However, in some examples, the bilateral stimulation may be delivered to different nerves (e.g., hypoglossal nerve, IHM-innervating nerve, scalene muscle-innervating nerves) and/or muscles, such as stimulating one nerve (e.g., hypoglossal nerve) on a left side of the body while stimulating another nerve (e.g., IHM-innervating nerve) on a right side of the body, or vice versa.

In some examples, the bilateral parameter 2212 may be implemented in a manner complementary with the alternating parameter 2232, simultaneous parameter 2234, or demand parameter 2236 of multiple function 2230, as further described below.

In some examples, the stimulation portion 2200 may comprise a multiple function 2230 by which various stimulation parameters may be implemented in dynamic arrangements. In some such examples, the stimulation portion 2200 may comprise an alternating parameter 2232 by which stimulation of one target tissue (e.g., hypoglossal nerve) may be alternated with stimulation of at least one other target tissue (e.g., IHM-innervating nerve). However, the alternating parameter 2232 also may be applied in combination with the bilateral parameter 2212 to apply stimulation to the same nerve (or different nerves or muscles) on opposite sides of the body in which stimulation may be applied on a left side of the body and then applied on the right side of the body in an alternating manner.

In some examples, the stimulation portion 2200 may comprise a simultaneous parameter 2234 by which stimulation may be applied simultaneously to at least two different target tissues. In some examples, the at least two different target tissues comprise two different nerves, such as the hypoglossal nerve and an IHM-innervating nerve, in some examples. However, in some examples, the at least two different target tissues may comprise two different locations along the same nerve or two different fascicles of the same nerve or muscles. In some examples, the simultaneous parameter 2234 may apply stimulation per bilateral parameter 2212 simultaneously on opposite sides of the body to the same nerve (e.g., hypoglossal nerve) or different nerves.

In some examples, the stimulation portion 2200 may comprise a demand parameter 2236 by which stimulation may be applied to at least one nerve (and/or muscles) on a demand basis. For example, stimulation may be applied to one nerve (e.g., hypoglossal nerve) which may be sufficient to achieve the patient metric (e.g., therapy outcome and/or usage) for most nights, for most sleeping positions (e.g., left and right lateral decubitis, prone), etc. but may become insufficient for some nights (e.g., after consuming alcohol or certain drugs which relax upper airway muscles), some sleeping positions (e.g., supine). In the latter situation, in order to achieve the target patient metric, via the demand parameter 2236, stimulation of a different nerve (e.g., IHM-innervating nerve, or scalene muscle-innervating nerve) or muscle (e.g., IHM) may be implemented in addition to, or instead of, stimulation of the first nerve (e.g., hypoglossal nerve) which was previously being stimulated. In some examples, the first or primary nerve being stimulated may be a nerve other than the hypoglossal nerve such as, but not limited to, the IHM-innervating nerve and/or at least one IHM.

In some examples, the stimulation portion 2200 also may further implement at least some aspects of the stimulation described throughout examples of the present disclosure and/or some aspects of the parameters 2210, 2212, 2230 of stimulation portion 2200 according to at least one of a closed loop parameter 2220, open loop parameter 2222, and nightly titration parameter 2224.

In some examples, the stimulation portion 2200 comprises a closed loop parameter 2220 to deliver stimulation therapy based on sensed patient physiologic information and/or other information (e.g., environmental, temporal, etc.). In some such examples, via the closed loop parameter 2220 the sensed information may be used to control the particular timing of the stimulation according to respiratory information, in which the stimulation pulses are triggered by or synchronized with specific portions (e.g., inspiratory phase) of the patient's respiratory cycle(s). In some such examples and as previously described, this respiratory information and/or other information used with the closed loop parameter 2220 may be determined via the sensors, sensing elements, devices, sensing portions, as previously described in association with at least FIG. 25.

In some examples, with or without timing stimulation relative to sensed respiratory information, the closed loop mode (2220) may comprise delivering stimulation therapy in response to sensed disease burden, such as the average number of apnea events per a time period, such as an apnea-hypopnea index (AHI) of average number of apnea events per hour. For example, for some periods of time within a nightly treatment period or over the course of several days/weeks, a patient may experience few sleep disordered breathing events (e.g., apnea events), such that stimulation therapy may be not delivered. However, upon the patient beginning to experience sleep disordered breathing at a level high enough to warrant stimulation therapy, then via the closed loop parameter 2220, stimulation therapy may be delivered to achieve a therapy outcome (e.g., AHI of 5 or less, in some examples and/or usage meeting a criteria (e.g., number of nights per week, number of hours per night, etc.).

In some examples the stimulation portion 2200 comprises an open loop parameter (e.g., 2222 in FIG. 26) by which stimulation therapy (e.g., “use”) is applied without a feedback loop of sensed physiologic information. In some such examples, in an open loop mode the stimulation therapy is applied during a treatment period without (e.g., independent of) information sensed regarding the patient's sleep quality, sleep state, respiratory phase, AHI, etc. In some such examples, in an open loop mode the stimulation therapy is applied during a treatment period without (e.g., independent of) particular knowledge of the patient's respiratory cycle information.

In some examples the stimulation portion 2200 comprises a nightly titration parameter 2224 by which an intensity of stimulation therapy may be titrated (e.g., adjusted) to be more intense (e.g., higher amplitude, greater frequency, and/or greater pulse width) or to be less intense within a nightly treatment period.

In some such examples, the nightly titration parameter 2224 may be implemented as automatic titration while in some examples, the titration parameter may be implemented via manual titration by a patient (or clinician). In some examples, the titration parameter may be implemented via combination of patient/manual titration and automatic titration to guide the patient in a manner complementary with their manual titration.

In some such examples and as previously described, such titration may be implemented at least partially based on sleep quality, which may be obtained via sensed physiologic information, in some examples. It will be understood that such examples may be employed with synchronizing stimulation to sensed respiratory information (e.g., closed loop stimulation) or may be employed without synchronizing stimulation to sensed respiratory information (e.g., open loop stimulation).

In some examples, at least some aspects of the titration parameter 2224 of the stimulation portion 2200 may comprise (and/or may be implemented) in a manner complementary with and/or via at least some of substantially the same features and attributes as described in U.S. Pat. No. 8,938,299, entitled “SYSTEM FOR TREATING SLEEP DISORDERED BREATHING”, issued Jan. 20, 2015, and which is hereby incorporated by reference in its entirety.

FIG. 27A is a block diagram schematically representing an example control portion. In some examples, the control portion 2100 includes a controller (e.g., processor) 2102 and a memory 2104. In some examples, the control portion 2100 provides one example implementation of a control portion forming a part of, implementing, and/or managing any one of devices and/or methods, or portions thereof (e.g., assemblies, circuitry, managers, engines, functions, parameters, respiration determination elements, stimulation elements, IPGs, sensors, electrodes, modules) as represented in various examples throughout the present disclosure in association with FIGS. 1A-26.

The control portion 2100 may include circuitry components and wiring appropriate for generating desired stimulation signals (e.g., converting energy provided by the power source into a desired stimulation signal), for example in the form of the care engine 2109. In some examples, the control portion 2100 may include telemetry components for communication with external devices. For example, the control portion 2100 may include a transmitter that transforms electrical power into a signal associated with transmitted data packets, a receiver that transforms a signal into electrical power, a combination transmitter/receiver (or transceiver), an antenna (e.g., an inductive telemetry antenna), etc.

In general terms, the controller 2102 of the control portion 2100 comprises an electronics assembly 2106 (e.g., at least one processor, microprocessor, integrated circuits and logic, etc.) and associated memories or storage devices. The controller 2102 is electrically couplable to, and in communication with, the memory 2104 to generate control signals to direct operation of at least some aspects of any one of above-mentioned devices and/or methods (or portions thereof), as represented throughout the present disclosure. In some examples, these generated control signals include, but are not limited to, employing the stimulation at or near the target location of the IHM-innervating nerve. The control signals may be a software program stored on the memory 2104 (which may be stored on another storage device and loaded onto the memory 2104), and executed by the electronics assembly 2106. In some examples, the control signals also may at least identify respiration information, cardiac information, and/or upper airway obstruction, and optionally, the body position and/or sleep state, among other sensed parameters. In addition, and in some examples, these generated control signals include, but are not limited to, employing the care engine 2109 stored in the memory 2104 to at least manage care provided to the patient, for example therapy for SDB (and/or other therapies, such as cardiac), with such care in at least some examples including stimulating an IHM-related tissue and/or identifying a target location of the IHM-related tissue, such as an IHM-innervating nerve.

In response to or based upon commands received via a user interface (e.g., user interface 2240 in FIG. 28), sensor signals, and/or via machine readable instructions, controller 2102 generates control signals as described above in accordance with examples of the present disclosure. In some examples, controller 2102 is embodied in a general purpose computing device while in some examples, controller 2102 is incorporated into or associated with at least some aspects of any one of above-mentioned devices and/or methods (or portions thereof) as described throughout the various examples of the present disclosure.

For purposes of this application, in reference to the controller 2102, the term “processor” shall mean a presently developed or future developed processor (or processing resources) that executes machine readable instructions contained in a memory. In some examples, execution of the machine readable instructions, such as those provided via memory 2104 of control portion 2100 cause the processor to perform the above-identified actions, such as operating controller 2102 to implement the sensing, monitoring, identifying the upper airway obstruction, stimulating, and/or treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 2104. In some examples, the machine readable instructions may comprise a sequence of instructions, or the like. In some examples, memory 2104 comprises a computer readable tangible medium providing non-volatile storage of the machine readable instructions executable by a processor of controller 2102. In some examples, the computer readable tangible medium may sometimes be referred to as, and/or comprise at least a portion of, a computer program product. In some examples, hard wired circuitry may be used in place of or in combination with machine readable instructions to implement the functions described. For example, controller 2102 may be embodied as part of at least one application-specific integrated circuit (ASIC), at least one field-programmable gate array (FPGA), and/or the like. In some examples, the controller 2102 is not limited to any specific combination of hardware circuitry and machine readable instructions, nor limited to any particular source for the machine readable instructions executed by the controller 2102.

In some examples, control portion 2100 may be entirely implemented within or by a stand-alone device.

In some examples, the control portion 2100 may be partially implemented in one of the sensors, sensing element, respiration determination elements, monitoring devices, stimulation devices, etc. and partially implemented in a computing resource (e.g., at least one external resource) separate from, and independent of, the IMD (or portions thereof) but in communication with the IMD (or portions thereof). For instance, in some examples, control portion 2100 may be implemented via a server accessible via the cloud and/or other network pathways. In some examples, the control portion 2100 may be distributed or apportioned among multiple devices or resources such as among a server, an apnea treatment device (or portion thereof), and/or a user interface.

In some examples, control portion 2100 includes, and/or is in communication with, a user interface 2240 as shown in FIG. 28.

FIG. 27B is a diagram schematically illustrating at least some example arrangements of a control portion by which the control portion 2100 (FIG. 27A) may be implemented. In some examples, control portion 2120 is entirely implemented within or by an IPG 2125, which has at least some of substantially the same features and attributes as IPG, as previously described throughout the present disclosure. In some examples, control portion 2120 is entirely implemented within or by a remote control 2130 (e.g., a programmer) external to the patient's body, such as a patient control 2132 and/or a physician control 2134. In some examples, the control portion 2120 is partially implemented in the IPG 2125 and partially implemented in the remote control 2130 (at least one of patient control 2132 and physician control 2134).

FIG. 28 is a block diagram schematically representing a user interface. In some examples, user interface 2240 forms part of and/or is accessible via a device external to the patient and by which the IPG and/or other portion of an IMD may be at least partially controlled and/or monitored. The external device which hosts user interface 2240 may be a patient remote (e.g., 2132 in FIG. 27B), a physician remote (e.g., 2134 in FIG. 27B) and/or a clinician portal. In some examples, user interface 2240 comprises a user interface or other display that provides for the simultaneous display, activation, and/or operation of at least some aspects of any one of above-mentioned devices and/or methods (or portions thereof) as described in connection with FIGS. 1A-27B. In some examples, at least some portions or aspects of the user interface 2240 are provided via a graphical user interface (GUI), and may comprise a display 2244 and input 2242.

FIG. 28 is a block diagram 2350 which schematically represents some example implementations by which an implantable device may communicate wirelessly with external circuitry outside the patient. As described above, the controller and/or control portion of at least one IPG 2360 illustrated in FIG. 28 may be implemented by components of the IPG 2360, components of external devices (e.g., mobile device 2370, patient remote control 2374, a clinician programmer 2376, and a patient management tool 2380), and various combinations thereof. As shown in FIG. 28, in some examples, the IPG 2360 may communicate with at least one of patient application 2372 on a mobile device 2370, a patient remote control 2374, a clinician programmer 2376, and a patient management tool 2380. The patient management tool 2380 may be implemented via a cloud-based portal 2383, the patient application 2372, and/or the patient remote control 2374. Among other types of data, these communication arrangements enable the IPG 2360 to communicate, display, manage, etc., the therapy provided, as well as to allow for adjustment to the various elements, portions, etc., of the example devices and methods if and where desired. In some examples, the various forms of therapy provided may be displayed to a patient and/or clinician via one of the above-described external devices.

Various examples of the present disclosure are directed to identifying and accessing a target location of an IHM-related tissue from which stimulations cause movement of the thyroid cartilage inferiorly and, optionally, the hyoid bone inferiorly, for promoting upper airway patency. In some examples, the stimulation at the target location of the IHM-related tissue may be used to treat sleep apnea, such as OSA. In some examples, the identification and access of the target location of the IHM-related tissue may be performed using an access approach and by verifying stimulation activates the physiological response sufficient to promote upper airway patency.

The various ranges provided herein include the stated range and any value or sub-range within the stated range. Furthermore, when “about” is utilized to describe a value, this includes, refers to, and/or encompasses variations (up to +/−10%) from the stated value.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method, comprising:
identifying a target location for stimulating an infrahyoid muscle-related tissue to promote patency of an upper airway of a patient.

2. The method of claim 1, wherein the infrahyoid muscle-related tissue is an infrahyoid muscle-innervating nerve, and the target location extends distally from a superior root portion of the infrahyoid muscle-innervating nerve and innervates at least one infrahyoid muscle.

3-7. (canceled)

8. The method of claim 1, wherein identifying the target location associated with the infrahyoid muscle-related tissue comprises:
locating at least one stimulation element at a first location to stimulate the infrahyoid muscle-related tissue; and
verifying the stimulation at the first location causes activation of at least one infrahyoid muscle.

9. (canceled)

10. The method claim 8, further comprising:
moving the at least one stimulation element to a second location in response to at least one of:
the stimulation not causing activation of the at least one at least one infrahyoid muscle; and
the stimulation causing activation of the at least one at least one infrahyoid muscle without causing a physiological response of at least one upper airway patency-related tissue; and
the stimulation causing activation of the at least one at least one infrahyoid muscle that causes the physiological response of at least one upper airway patency-related tissue below a threshold.

11-13. (canceled)

14. The method of claim 1, wherein identifying the target location comprises:
providing an incision at or near a clavicle of the patient at a level associated with the omohyoid muscle;
retracting the omohyoid muscle superiorly; and
locating at least one stimulation element at the target location at or near the infrahyoid muscle-related tissue.

15-36. (canceled)

37. A device comprising:
at least one implantable stimulation element to be in stimulating relation to an infrahyoid muscle-related tissue; and
optionally comprising a control portion to stimulate, via the at least one implantable stimulation element, the infrahyoid muscle-related tissue to promote patency of an upper airway of a patient.

38. The device of claim 37, wherein the infrahyoid muscle-related tissue is selected from:
an infrahyoid muscle-innervating nerve; and
at least one infrahyoid muscle.

39. The device of claim 38, wherein the infrahyoid muscle comprises at least the sternothyroid muscle.

40. The device of claim 38, wherein the infrahyoid muscle comprises the sternothyroid muscle and at least a portion of the sternohyoid muscle.

41. The device of claim 37, wherein the control portion is arranged with the at least one stimulation element to stimulate the infrahyoid muscle-related tissue at a first location to activate at least one infrahyoid muscle.

42. The device of claim 41, wherein the stimulation element is movable to other locations.

43. The device of claim 41, wherein the activation of the at least one infrahyoid muscle causes a physiological response of at least one upper airway patency-related tissue.

44. The device of claim 43, wherein the physiological response comprises:
movement of the thyroid cartilage inferiorly; and/or
movement of the hyoid bone inferiorly.

45. The device of claim 37, further comprising a stimulation lead on which the least one stimulation element is supported, in a position extending between an implantable pulse generator and a stimulating relation to the target location of the infrahyoid muscle-related tissue.

46. The device of claim 37, wherein the least one stimulation element comprises an electrode cuff to at least partially enclose at least a portion of the infrahyoid muscle-related tissue at the target location.

47. The device of claim 37, wherein the least one stimulation element comprises a U-shaped head carrying at least one stimulation electrode, wherein the U-shaped head is to at least partially surround at least a portion of the infrahyoid muscle-related tissue at the target location.

48. The device of claim 37, wherein the least one stimulation element comprises at least one axial electrode array.

49. The device of claim 37, wherein the least one stimulation element comprises at least one array of spaced apart stimulation electrodes supported by a lead portion.

50. The device of claim 37, wherein the least one stimulation element comprises a paddle-style body supporting at least one stimulation electrode.

51. The device of claim 37, further comprising at least one fixation element to anchor the at least one stimulation element to tissue.
